(19) United States
(12) Patent Application Publication
(10) Pub. No.: US 2013/0261167 A1
(43) Pub. Date: Oct. 3, 2013

(54) ANCCA as a Diagnostic Biomarker and Therapeutic Target for Breast Cancers

(71) Applicants: Hong-Wu Chen, Sacramento, CA (US); June X. Zou, Sacramento, CA (US)

(72) Inventors: Hong-Wu Chen, Sacramento, CA (US); June X. Zou, Sacramento, CA (US)

(73) Assignee: The Regents Of The University Of California, Oakland, CA (US)

(21) Appl. No.: 13/825,309

(22) PCT Filed: Sep. 23, 2011
(86) PCT No.: PCT/US2011/052998

§ 371 Date: Jun. 13, 2013

Related U.S. Application Data
(60) Provisional application No. 61/385,962, filed on Sep. 23, 2010.

(51) Int. Cl. G01N 33/68; C12N 15/113; C12Q 1/68

(52) U.S. Cl. CPC G01N 33/6875; C12Q 1/6886; C12N 15/113
USPC 514/44 A; 435/7.4; 435/6.12; 435/375

(57) ABSTRACT

The present invention provides methods of diagnosing breast cancer, confirming a diagnosis of breast cancer, predicting the survival of a breast cancer patient, and providing a therapeutic target of treatment in a breast cancer patient by measuring relatively increased expression levels of ANCCA. Expression levels of ANCCA can be measured be determining the levels mRNA or protein. The expression levels can be compared to a predetermined threshold level or to a control, e.g., a positive or negative control.

### CROSS-REFERENCE TO RELATED APPLICATIONS

This application is a U.S. national phase under 35 U.S.C. §371 of International Application No. PCT/US2011/052998, filed on Sep. 23, 2011, which claims the benefit under 35 U.S.C. §119(e) of U.S. Provisional Application No. 61/385,962, filed on Sep. 23, 2010, both of which are hereby incorporated herein by reference in their entirety for all purposes.

### STATEMENT OF GOVERNMENTAL SUPPORT

This work was supported in part by Grant Nos. R01DK060019 and R01CA113860, both awarded by the National Institutes of Health. The Government has certain rights in this invention.

### FIELD OF THE INVENTION

The present invention relates to use of ANCCA, an AAA+ ATPase nuclear coactivator for estrogen receptor-α (ERα) and androgen receptor as a diagnostic, prognostic and therapeutic target in breast cancers.

### BACKGROUND OF THE INVENTION

Functional genomics and gene profiling studies of breast cancer continue to improve our prediction of clinical outcome and selection of therapeutics as well as our understanding of tumor biology, by subtyping the tumors on their molecular profiles. Among the subtypes, the luminal/estrogen receptors positive (ER+) tumors present themselves as more differentiated, less aggressive, and highly responsive to endocrine therapy. Tumors overexpressing human epidermal growth factor receptor 2 (HER2), although often more aggressive, are responsive to therapeutics targeting the growth factor receptors. The triple negative (e.g., negative for detection of ER, progesterone receptor, and HER2-negative) breast cancers or TNBCs, however, remain a major challenge for the development of effective therapeutics and identification of risk factors (1). Recent expression array studies (2, 3) characterize most TNBCs as being basal-like due to an expression profile containing high levels of basal cell cytokeratins, similar to those of myoepithelial cells. Although poorly understood, one striking feature of TNBCs is the high expression of proliferation signature genes. Unlike ER-positive or HER2-high subtypes, no molecular markers have been defined that underpin TNBC development or effectively guide their clinical treatment. As a result, conventional chemotherapy remains the mainstay for them. Therefore, there is an urgent need for the identification of risk factors and new treatment options for triple negative tumors.

Chromatin coregulators, particularly in the forms of histone modifying and chromatin remodeling enzymes, have recently emerged as important players in tumorigenesis (4-6). We recently identified a previously uncharacterized gene product dubbed as ANCCA (for AAA+ nuclear coregulator cancer associated) as both a direct target and an activator of the proto-oncogene AIB1 (also known as ACTR and SRC-3) (7, 8). ANCCA, a novel member of the AAA+ ATPase proteins, also possesses a bromodomain. We found that high levels of ANCCA are expressed in breast cancer and prostate cancer cells and that RNAi-mediated knockdown of ANCCA strongly inhibits hormone-dependent cancer cell proliferation (8, 9). We also demonstrated that ANCCA acts as a coactivator of ERa and androgen receptor (AR) to mediate estrogen- or androgen-induced expression of specific subsets of genes involved in proliferation and survival of cancer cells. Both the AAA+ ATPase and bromodomains are required for ANCCA to serve as a transcriptional coregulator of the receptors (8). Our further study indicated that one major mechanism of ANCCA function is to facilitate the assembly of a histone modifying protein complex at the chromatin (8). In this study, we investigated ANCCA expression at both protein and transcript levels in multiple sets of human breast cancer specimens and found that ANCCA is overexpressed in the majority of the tumors. High levels of ANCCA directly correlate with poor survival and disease recurrence in the patients. We also present results that high ANCCA is strongly associated with triple-negative tumors and that aberrant ANCCA expression in TNBC cells controls multiple oncogenic pathways for cancer cell proliferation and survival.

### SUMMARY OF THE INVENTION

In one aspect, the invention provides methods of determining the presence of breast cancer in a subject in need thereof. In some embodiments, the methods comprise:
a) measuring the expression level of ATPase family AAA domain-containing protein 2 (ANCCA) in a breast tissue suspected of being cancerous;
b) comparing the expression level of ANCCA in the breast tissue to a threshold level of expression, wherein an expression level of ANCCA that is greater than the threshold level of expression is indicative of the presence of breast cancer in the subject.

In a further aspect, the invention provides methods of prognosticating, predicting or diagnosing the survival of a subject with breast cancer. In some embodiments, the methods comprise:
a) measuring the expression level of ANCCA in a breast tissue suspected of being cancerous;
b) comparing the expression level of ANCCA in the breast tissue to a threshold level of expression, wherein an expression level of ANCCA that is greater than the threshold level of expression is indicative of the decreased length of survival of the subject (e.g., a poor prognosis). Similarly, an expression level of ANCCA that is less than the threshold level of expression is indicative of the increased length of survival of the subject (e.g., a favorable prognosis).

In another aspect, the invention provides methods of determining the likelihood or risk of recurrence of breast cancer in a subject. In some embodiments, the methods comprise:
a) measuring the expression level of ANCCA in a breast tissue suspected of being cancerous or in a breast cancer tumor;
b) comparing the expression level of ANCCA in the breast tissue or breast cancer tumor to a threshold level of expression, wherein an expression level of ANCCA that is greater than the threshold level of expression indicates a relatively higher likelihood or risk of breast cancer recurrence (e.g., a poor prognosis). Similarly, an expression level of ANCCA that is less than the threshold level of expression indicates a relatively lower likelihood or risk of breast cancer recurrence (e.g., a favorable prognosis). In some embodiments, the subject is in remission from breast cancer.

In a related aspect, the invention provides methods of predicting the presence or occurrence of breast cancer metastasis in a subject with breast cancer, comprising
a) measuring the expression level of ANCCA in a tissue suspected of being cancerous;
b) comparing the expression level of ANCCA in the tissue to a threshold level of expression, wherein an expression level of ANCCA that is greater than the threshold level of expression is indicative of or associated with the presence or occurrence of breast cancer metastasis in the subject. In some embodiments, the tissue is breast tissue.

In a further aspect, the invention provides methods of monitoring the progression of a breast cancer. In some embodiments, the methods comprise:
a) measuring the expression level of ANCCA in a breast tissue suspected of being cancerous;
b) comparing the expression level of ANCCA in the breast tissue to a threshold level of expression, wherein an expression level of ANCCA that is greater than the threshold level of expression or greater than a previously measured level of expression is indicative of progression of the breast cancer in the subject (e.g., a poor prognosis). In various embodiments, the level of ANCCA expression may be 10%, 15%, 20%, 25%, or greater percentage, greater or higher than a previously measured expression level. Similarly, an expression level of ANCCA that is less than the threshold level of expression or less than a previously measured level of expression is indicative of lack of progression, stabilization and/or remission of the breast cancer in the subject (e.g., a favorable prognosis). In various embodiments, the level of ANCCA expression may be 10%, 15%, 20%, 25%, or greater percentage, less or lower than a previously measured expression level.

In various embodiments, the threshold level is equivalent to the level of ANCCA expression in a normal or non-cancerous tissue. In various embodiments, the threshold level is equivalent to the level of ANCCA expression in a population of individuals diagnosed with breast cancer. In various embodiments, the threshold level is equivalent to the level of ANCCA expression in a population of individuals diagnosed with stage I breast cancer. In various embodiments, the threshold level is equivalent to the level of ANCCA expression in a population of individuals diagnosed with stage II breast cancer. In various embodiments, the threshold level is equivalent to the level of ANCCA expression in a population of individuals diagnosed with stage III breast cancer. In various embodiments, the threshold level is equivalent to the level of ANCCA expression in a population of individuals diagnosed with grade 1 breast cancer. In various embodiments, the threshold level is equivalent to the level of ANCCA expression in a population of individuals diagnosed with grade 2 breast cancer. In various embodiments, the threshold level is equivalent to the level of ANCCA expression in a population of individuals diagnosed with grade 3 breast cancer. In various embodiments, the threshold level is equivalent to the level of ANCCA expression in a population of individuals diagnosed with metastatic breast cancer.

In some embodiments, the tissue is an epithelial tissue.

In some embodiments, the tissue is from a biopsy. In some embodiments, the methods further comprise the step of obtaining a biological sample, e.g., of breast tissue or from a tissue suspected of containing breast cancer metastasis. In various embodiments, the tissue is from a primary tumor.

In some embodiments, the subject is exhibiting symptoms of breast cancer. In some embodiments, the subject has a familial history of breast cancer. In some embodiments, the subject has received a preliminary diagnosis or a diagnosis of breast cancer. In some embodiments, the subject has not or does not respond to tamoxifen therapy. In some embodiments, the subject has an estrogen receptor (ER)-negative breast cancer. In some embodiments, the subject has an ERα- and PR-negative breast cancer. In some embodiments, the subject has triple-negative breast cancer (i.e., is negative for detection of estrogen receptors (ER−), progesterone receptors (PR−), and HER2 (HER2−)). In some embodiments, the subject has a breast cancer that also overexpresses or expresses elevated levels of Ki-67. In some embodiments, the subject has a breast cancer that is BRCA1 defective or has a defective BRCA1 gene.

In some embodiments, the subject is in remission from breast cancer. In some embodiments, the subject is human.

In some embodiments, the protein expression level of ANCCA is measured. In some embodiments, the ANCCA protein has an amino acid sequence having at least 90%, 91%, 92%, 93%, 94%, 95%, 96%, 97%, 98%, 99% sequence identity to GenBank Accession No. NP_{—}054828.2 (SEQ ID NO:49). In some embodiments, the ANCCA protein has the amino acid sequence of GenBank Accession No. NP_{—}054828.2 (SEQ ID NO:49).

In some embodiments, the transcription expression level of ANCCA is measured. In some embodiments, the nucleic acid sequence encoding ANCCA has a nucleic acid sequence having at least 90%, 91%, 92%, 93%, 94%, 95%, 96%, 97%, 98%, 99% sequence identity to GenBank Accession No. NM_{—}014109.3 (SEQ ID NO:50). In some embodiments, the nucleic acid sequence encoding ANCCA has a nucleic acid sequence of GenBank Accession No. NM_{—}014109.3 (SEQ ID NO:50).

In some embodiments, the methods further comprise the step of providing a therapeutic or preventative regime to the subject, e.g., based on the predictive, diagnostic and/or prognostic results of measuring the expression levels of ANCCA.

In various embodiments, the therapeutic or preventative regime comprises reducing or inhibiting the activity of ANCCA. In some embodiments, the ATPase activity of ANCCA is reduced or inhibited. In some embodiments, the expression of ANCCA is reduced or inhibited. In some embodiments, the expression of ANCCA is inhibited at the transcriptional level, e.g., by administration to the subject of an inhibitory nucleic acid that specifically hybridizes to ANCCA. In some embodiments, the expression of ANCCA is inhibited at the protein level, e.g., by administration to the subject of an antibody that specifically binds to ANCCA.

In another aspect, the invention provides methods of reducing, inhibiting or preventing the growth or proliferation of a breast cancer cell. In some embodiments, the methods comprise contacting the cell with an agent that inhibits the activity of ANCCA, wherein the agent binds to ANCCA or prevents or inhibits the expression of ANCCA, thereby inhibiting the activity of ANCCA, thereby reducing, inhibiting or preventing the growth or proliferation of the breast cancer cell.

In another aspect, the invention provides methods of reducing, inhibiting or preventing the metastasis of a breast cancer in a subject in need thereof. In some embodiments, the methods comprise contacting the cell with an agent that inhibits the activity of ANCCA, wherein the agent binds to ANCCA or prevents or inhibits the expression of ANCCA, thereby inhibiting the activity of ANCCA, thereby reducing, inhibiting or preventing the metastasis of the breast cancer.

In another aspect, the invention provides methods of reducing, inhibiting or preventing the progression of a breast cancer in a subject in need thereof (e.g., from stage I to stage II, from stage II to stage III, from stage III to stage IV, from metastasizing at any stage, etc.). In some embodiments, the methods comprise contacting the cell with an agent that inhibits the activity of ANCCA, wherein the agent binds to ANCCA or prevents or inhibits the expression of ANCCA, thereby inhibiting the activity of ANCCA, thereby reducing, inhibiting or preventing the progression of the breast cancer.

In another aspect, the invention provides methods of reducing, inhibiting or preventing the growth or proliferation of a breast cancer cell. In some embodiments, the methods comprise contacting the cell with an inhibitory nucleic acid that specifically hybridizes to ANCCA, wherein the expression of ANCCA is reduced or inhibited by the inhibitory nucleic acid, thereby reducing, inhibiting or preventing the growth or proliferation of the breast cancer cell.

In another aspect, the invention provides methods of reducing, inhibiting or preventing the metastasis of a breast cancer in a subject in need thereof. In some embodiments, the methods comprise contacting a breast cancer cell with an inhibitory nucleic acid that specifically hybridizes to ANCCA, wherein the expression of ANCCA is reduced or inhibited by the inhibitory nucleic acid, thereby reducing, inhibiting or preventing the metastasis of the breast cancer.

In another aspect, the invention provides methods of reducing, inhibiting or preventing the progression of a breast cancer in a subject in need thereof (e.g., from stage I to stage II, from stage II to stage III, from stage III to stage IV, from metastasizing at any stage, etc.). In some embodiments, the methods comprise contacting a breast cancer cell with an inhibitory nucleic acid that specifically hybridizes to ANCCA, wherein the expression of ANCCA is reduced or inhibited by the inhibitory nucleic acid, thereby reducing, inhibiting or preventing the metastasis of the breast cancer.

In some embodiments, the breast cancer cell is in vitro. In some embodiments, the breast cancer cell is in vivo.

In some embodiments, the breast cancer cell is an estrogen receptor (ER)-negative breast cancer cell. In some embodiments, the breast cancer cell is a triple-negative breast cancer cell.

In some embodiments, the inhibitory nucleic acid specifically binds, anneals or hybridizes to a nucleic acid sequence having at least 90%, 91%, 92%, 93%, 94%, 95%, 96%, 97%, 98%, 99% or 100% sequence identity to GenBank Accession No. NM_{—}014109.3 (SEQ ID NO:50) or a nucleic acid encoding an amino acid sequence having at least 90%, 91%, 92%, 93%, 94%, 95%, 96%, 97%, 98%, 99% or 100% sequence identity to GenBank Accession No. NP_{—}054828.2 (SEQ ID NO:49).

In some embodiments, the inhibitory nucleic acid is selected from an antisense RNA, a ribozyme, a short inhibitory RNA (siRNA), and a micro RNA (mRNA).

### DEFINITIONS

Structurally, “ATPase family, AAA domain containing 2,” “ANCCA” or “ATAD2” refers to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 400, or more amino acids, or over the full-length, to an amino acid sequence encoded by an ANCCA nucleic acid (see, e.g., GenBank Accession No. NM_{—}014109.3 (SEQ ID NO:50); (2) bind to antibodies, e.g., polyclonal antibodies, raised against an immunogen comprising an amino acid sequence of an ANCCA polypeptide (see, e.g., GenBank Accession No. NP_{—}054828.2 (SEQ ID NO:49)); or an amino acid sequence encoded by an ANCCA nucleic acid (e.g., ANCCA polynucleotides described herein), and conservatively modified variants thereof; (3) specifically hybridize under stringent hybridization conditions to an anti-sense strand corresponding to a nucleic acid sequence encoding an ANCCA protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to an ANCCA nucleic acid (e.g., ANCCA polynucleotides, as described herein, and ANCCA polynucleotides that encode ANCCA polypeptides, as described herein).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine I, Lysine (K);
5) Isoleucine (I), Leucine (L), Methionine (M), Valine (V);
6) Phenylalanine (F), Tyrosine (Y), Tryptophan (W); and
7) Serine (S), Threonine (T)
(see, e.g., Creighton, Proteins (1984)).

An antibody refers to either a polyclonal or monoclonal antibody that is able to recognize a specified protein. Antibodies may be generated that are able to recognize either an entire protein, or short peptide sequences within a full length protein.

The terms “bind(s) specifically” or “specifically bind(s)” or “attached” or “attaching” refers to the preferential association of an anti-ANCCA antibody, in whole or part, with a cell or tissue bearing a particular target epitope (i.e., an ANCCA polypeptide) in comparison to cells or tissues lacking that target epitope. It is, of course, recognized that a certain degree of non-specific interaction may occur between an antibody and a non-target epitope. Nevertheless, specific binding, may be distinguished as mediated through specific recognition of the target epitope. Typically specific binding results in a much stronger association between the delivered molecule and an entity (e.g., an assay well or a cell) bearing the target epitope than between the bound antibody and an entity (e.g., an assay well or a cell) lacking the target epitope. Specific binding typically results in greater than about 10-fold and most preferably greater than 100-fold increase in amount of bound anti-ANCCA antibody (per unit time) to a cell or tissue bearing the target epitope as compared to a cell or tissue lacking the target epitope. Specific binding between two entities generally means an affinity of at least 10⁶ M−1. Affinities greater than 10⁸ M−1 are preferred. Specific binding can be determined using any assay for antibody binding known in the art, including Western Blot, ELISA, flow cytometry, immunohistochemistry.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., an ANCCA polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50-100 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to ANCCA nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc.

Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)).

Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “threshold level” refers to a representative or predetermined expression level of ANCCA polypeptide or mRNA. The threshold level can represent expression detected in a sample from a normal control, i.e., from non-cancerous tissue, usually of the same tissue type of the biological sample subject to testing. The threshold level can be determined from an individual or from a population of individuals, e.g., known to not have breast cancer or known to have breast cancer of a predetermined stage, e.g., stage I, stage II, stage III, or metastatic breast cancer. In the present diagnostic methods, ANCCA expression levels, at the transcriptional or protein level, above the threshold level is generally indicative of the presence of cancer; expression levels below the threshold level are generally indicative of non-cancerous tissue.

The term “increased expression level” is generally made with reference to a predetermined threshold level or a level of expression from a normal or non-cancerous control. An increased expression level is determined when the level of expression in the test biological sample is at least about 10%, 25%, 50%, 75%, 100% (i.e., 1-fold), 2-fold, 3-fold, 4-fold or greater, in comparison to the predetermined threshold level of expression or the level of expression from a normal or non-cancerous control tissue. In determining an increased level of expression, usually the same tissue types are compared.

“Cancer” refers to mammalian (e.g., human) cancers and carcinomas, sarcomas, adenocarcinomas, lymphomas, leukemias, etc., including solid and lymphoid cancers. Of interest are epithelial cancers (e.g., carcinomas). The cancers subject to diagnosis and treatment by the present invention are associated with, correlated with and/or caused by the overexpression of ANCCA.

The terms “treating” and “treatment” and variants thereof refer to delaying the onset of, retarding or reversing the progress of, alleviating or preventing either the disease or condition to which the term applies, or one or more symptoms of such disease or condition (e.g., breast cancer). Treating and treatment encompass both therapeutic and prophylactic treatment regimens.

The term “individual,” “patient,”, “subject” interchangeably refer to a mammal, for example, a human, a non-human primate, a domesticated mammal (e.g., a canine or a feline), an agricultural mammal (e.g., equine, bovine, ovine, porcine), or a laboratory mammal (e.g., rattus, murine, lagomorpha, hamster).

The terms “decrease” or “reduce” or “inhibit” interchangeably refer to the detectable reduction of a measured response (e.g., ANCCA enzymatic activity, ANCCA expression levels). The decrease, reduction or inhibition can be partial, for example, at least 10%, 25%, 50%, 75%, or can be complete (i.e., 100%). The decrease, reduction or inhibition can be measured in comparison to a control. For example, decreased, reduced or inhibited responses can be compared before and after treatment. Decreased, reduced or inhibited responses can also be compared to an untreated control, or to a known value.

The term “substrate analog” refers to a compound (e.g., small molecules) that shares structural and/or functional similarity with an enzyme substrate, but unlike the enzyme substrate, the substrate analog inhibits the function of the enzyme upon binding. A substrate analog can have structural similarity to an enzyme substrate as measured on a 2-dimensional or 3-dimensional (electron densities, location of charged, uncharged and/or hydrophobic moieties) basis. A substrate analog can have functional similarity with an enzyme substrate inasmuch as the substrate analog binds to the enzyme. A substrate analog can be a competitive inhibitor.

A “compound that inhibits ANCCA activity” refers to any compound that inhibits ANCCA activity. The inhibition can be, for example, on the transcriptional, translational or enzymatic level. Accordingly, the compound can be in any chemical form, including nucleic acid or nucleotide, amino acid or polypeptide, monosaccharide or oligosaccharide, nucleotide sugar, or small organic molecule.

The terms “systemic administration” and “systemically administered” refer to a method of administering an agent or an antigen binding molecule that binds to ANCCA to sites in the body, including the targeted site of pharmaceutical action, via the circulatory system. Systemic administration includes, but is not limited to, oral, intranasal, rectal and parenteral (i.e., other than through the alimentary tract, such as intramuscular, intravenous, intra-arterial, transdermal and subcutaneous) administration.

The term “co-administer” and “co-administering” and variants thereof refer to the simultaneous presence of two or more active agents in the blood of an individual. The active agents that are co-administered can be concurrently or sequentially delivered.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

The phrase “consisting essentially of” and variants thereof refer to the genera or species of active agents expressly identified in a method or composition, as well as any excipients inactive for the intended purpose of the methods or compositions.

An “antigen binding molecule,” as used herein, is any molecule that can specifically or selectively bind to an antigen. A binding molecule may include an antibody or a fragment thereof. An anti-ANCCA binding molecule is a molecule that binds to the ANCCA antigen, such as an anti-ANCCA antibody or fragment thereof. Other anti-ANCCA binding molecules may also include multivalent molecules, multi-specific molecules (e.g., diabodies), fusion molecules, aptimers, avimers, or other naturally occurring or recombinantly created molecules. Illustrative antigen-binding molecules useful to the present methods include antibody-like molecules. An antibody-like molecule is a molecule that can exhibit functions by binding to a target molecule (See, e.g., Current Opinion in Biotechnology 2006, 17:653-658; Current Opinion in Biotechnology 2007, 18:1-10; Current Opinion in Structural Biology 1997, 7:463-469; Protein Science 2006, 15:14-27), and includes, for example, DARPins (WO 2002/020565), Affibody (WO 1995/001937), Avimer (WO 2004/044011; WO 2005/040229), and Adnectin (WO 2002/032925).

An “antibody” refers to a polypeptide of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ. Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (VL) and variable heavy chain (VH) refer to these regions of light and heavy chains, respectively. As used in this application, an “antibody” encompasses all variations of antibody and fragments thereof that possess a particular binding specifically, e.g., for tumor associated antigens. Thus, within the scope of this concept are full length antibodies, chimeric antibodies, humanized antibodies, human antibodies, unibodies, single domain antibodies or nanobodies, single chain antibodies (ScFv), Fab, Fab′, and multimeric versions of these fragments (e.g., F(ab′)₂) with the same binding specificity.

Typically, an immunoglobulin has a heavy and light chain. Each heavy and light chain contains a constant region and a variable region, (the regions are also known as “domains”). Light and heavy chain variable regions contain a “framework” region interrupted by three hypervariable regions, also called “complementarity-determining regions” or “CDRs”. The extent of the framework region and CDRs have been defined. See, Kabat and Wu, SEQUENCES OF PROTEINS OF IMMUNOLOGICAL INTEREST, U.S. Government Printing Office, NIH Publication No. 91-3242 (1991); Kabat and Wu, J. Immunol. (1991) 147(5):1709-19; and Wu and Kabat, Mol. Immunol. (1992) 29(9):1141-6. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

References to “VH” refer to the variable region of an immunoglobulin heavy chain, including an Fv, scFv, dsFv or Fab. References to “VL” refer to the variable region of an immunoglobulin light chain, including of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

The term “linker peptide” includes reference to a peptide within an antibody binding fragment (e.g., Fv fragment) which serves to indirectly bond the variable domain of the heavy chain to the variable domain of the light chain.

The term “specific binding” is defined herein as the preferential binding of binding partners to another (e.g., a polypeptide and a ligand (analyte), two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g., a randomly generated molecule lacking the specifically recognized site(s); or a control sample where the target molecule or antigen is absent).

With respect to antibodies of the invention, the term “immunologically specific” “specifically binds” refers to antibodies and non-antibody antigen binding molecules that bind to one or more epitopes of a protein of interest (e.g., ANCCA), but which do not substantially recognize and bind other molecules in a sample containing a mixed population of antigenic biological molecules.

The term “selectively reactive” refers, with respect to an antigen, the preferential association of an antibody, in whole or part, with a cell or tissue bearing that antigen and not to cells or tissues lacking that antigen (e.g., ANCCA). It is, of course, recognized that a certain degree of non-specific interaction may occur between a molecule and a non-target cell or tissue. Nevertheless, selective reactivity, may be distinguished as mediated through specific recognition of the antigen. Although selectively reactive antibodies bind antigen, they may do so with low affinity. On the other hand, specific binding results in a much stronger association between the antibody and cells bearing the antigen than between the bound antibody and cells lacking the antigen. Specific binding typically results in greater than 2-fold, preferably greater than 5-fold, more preferably greater than 10- or 20-fold and most preferably greater than 100-fold increase in amount of bound antibody (per unit time) to a cell or tissue bearing ANCCA as compared to a cell or tissue lacking ANCCA.

The term “immunologically reactive conditions” includes reference to conditions which allow an antibody generated to a particular epitope to bind to that epitope to a detectably greater degree than, and/or to the substantial exclusion of, binding to substantially all other epitopes. Immunologically reactive conditions are dependent upon the format of the antibody binding reaction and typically are those utilized in immunoassay protocols or those conditions encountered in vivo. See, e.g., Harlow & Lane, Using Antibodies, A Laboratory Manual (1998), for a description of immunoassay formats and conditions. Preferably, the immunologically reactive conditions employed in the methods of the present invention are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (i.e., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The term “therapeutic agent” includes any number of small organic compounds, polypeptides, peptides, and nucleic acids currently known or later developed to counteract or ameliorate cancer, e.g., breast cancer. In some embodiments, the therapeutic agent can be a chemotherapeutic agent, an anti-neoplastic agent, a cytotoxin or a radionuclide, where the therapeutic effect intended is, for example, the killing of a cancer cell.

The terms “effective amount” or “amount effective to” or “therapeutically effective amount” includes reference to a dosage of a therapeutic agent sufficient to produce a desired result.

The term “contacting” includes reference to placement in direct physical association.

The term “in vivo” includes reference to inside the body of the organism from which the cell was obtained. “Ex vivo” and “in vitro” means outside the body of the organism from which the cell was obtained.

The phrase “malignant cell” or “malignancy” refers to tumors or tumor cells that are invasive and/or able to undergo metastasis, i.e., a cancerous cell.

### BRIEF DESCRIPTION OF THE DRAWINGS

FIG. 1 illustrates Validation of anti-ANCCA antibody for IHC staining. A, IHC procedures were performed on a section of human breast cancer FFPE specimen with either anti-ANCCA antibody absorbed with a GST-fusion protein that was not used as an antigen for anti-ANCCA antibody generation (a), or with the antibody absorbed with GST-ANCCA (amino acids 2-264) that was used as antigen for the anti-ANCCA antibody production (b). B, Western blotting analysis using the anti-ANCCA antibody shows one major band of molecular weight approximately 170 kDa in the whole lysates of indicated breast cancer cell lines. Note the lower bands in the middle lane are likely derived from ANCCA protein as they are suppressed upon ANCCA-RNAi treatment of cells.

FIG. 2 illustrates immunohistochemistry (IHC) analysis of ANCCA expression in normal or cancerous human breast tissues. Representative images are shown for IHC score 0 with less than 1% of nuclei stained positive, in a histologically normal breast tissue (a) and in a tumor (b); for score 1 with <25% of positively stained nuclei in a tumor (c); for score 2 with 25-50% of positively stained nuclei a tumor (d); and for score 3 with >50% of positively stained nuclei in an ER-positive tumor (e) and an ER-negative tumor (f).

FIG. 3 illustrates ANCCA expression is significantly elevated in high grade tumors. A, representative images of anti-ANCCA IHC analysis of tumors in the TMA cores, which show different levels of ANCCA expression in different grades or normal breast tissue.

FIG. 4 illustrates ANCCA overexpression in breast cancer cell lines and its correlation with tumor grades. A, Western analysis of ANCCA in normal human mammary epithelial cells (HMEC) and in different breast cancer cell lines. Top panel, a short exposure; middle panel, a long exposure. B, representative anti-ANCCA IHC images of normal and tumor tissues with different grades. C, distribution of tumors with low (yellow bars), moderate (green bars) and high (orange bars) expression of ANCCA in breast carcinomas of different grades (P<0.05). D, box-and-whisker plot of ANCCA mRNA expression in tumors of different grades analyzed using the website oncomine.org.

FIG. 5 illustrates Box-and-whisker plots of ANCCA mRNA expression in normal and breast carcinoma tissues. The analysis was performed using the website oncomine.org. Data sets of Richardson A L et al., 2006 are from a study in Cancer Cell. 2006 February; 9(2):121-32; and data sets of Sorlie T et al., 2003 are from a study in Proc Natl Acad Sci USA. 2003 Jul. 8; 100(14):8418-23.

FIG. 6 illustrates Representative IHC images from adjacent sections of different tumor tissues for comparison of distribution of anti-ANCCA or anti-Ki67 IHC positive cells in the tumor.

FIG. 7 illustrates ANCCA overexpression correlates with poor outcomes. A and B, Kaplan-Meier analysis of ANCCA protein expression and disease-free survival (A) and disease recurrence (B). C. high ANCCA transcripts correlates with poor disease-free survival in tumor datasets from Ivshina et al., 2006, and associates with metastasis at five years after diagnosis in tumor datasets from Wang et al., 2005 and van'tVeer et al., 2002. The datasets were analyzed for the association using the website oncomine.org.

FIG. 8 illustrates Kaplan-Meier analysis of ANCCA protein expression and disease-free survival in ER-positive or ER-negative tumors.

FIG. 9 illustrates Elevated ANCCA is required for proliferation and survival of triple-negative breast cancer (TNBC) cells. A and B, Cells were either infected with equal titers of adeno-ANCCA or adeno-control (HCC1937) or incubated with siRNA targeting ANCCA or control sequence (MDA-MB 468). Western and cell proliferation assay were performed with cells harvested either 48 hrs after the treatments (for Western) or at indicated days. C, MDA-MB 231 cells were transfected with siRNA-ANCCA or siRNAcontrol and plated in soft agar for colony formation or Western. Colonies were stained with MTT four weeks later and counted. Representative images of colonies and their numbers from different treatments are shown. D, MDA-MB 231 cells were processed for TUNEL assay (left panel) or Western, 3 days after transfection with the siRNAs. For C and D independent samples t-test was used.

FIG. 10 illustrates ANCCA expression promotes anchorage-independent growth. MDA-MB 453 cells were transfected with ANCCA RNAi or control RNAi, plated in soft agar for colony formation. Representative images of colonies stained with MTT at 4× and 10× magnification, number of colonies formed from cells with different treatments obtained from three experiments, and Western analysis of ANCCA two days after siRNA transfection are shown.

FIG. 11 illustrates ANCCA is required for survival of triple negative breast cancer cells. MDAMB 231 cells were transfected with ANCCA RNAi or control RNAi, or mock treated, and processed for TUNEL assay. Representative images of TUNEL staining with cells with different treatments are shown.

FIG. 12 illustrates ANCCA controls key regulators of cell proliferation and survival pathways including oncogene EZH2. A, unsupervised hierarchical clustering of the expression of the top 20 genes (25 probesets, not including ANCCA) found to be co-expressed with ANCCA overexpression in a data set of 198 patients (12). Correlation coefficients (measured by Spearmann's test) for the similarity of each gene's expression to that of ANCCA expression are indicated herein. In the heat map, the magnitude of increased or decreased expression (relative to the mean) for each gene across the entire panel of samples is depicted by increasingly darker shades of red or blue, respectively; yellow is indicative of mean expression. Tumor ER status is indicated below by either blue bars for ER+ tumors (total of 134) or red bars for ER− tumors (total of 64). Also indicated at right is the inhibitory effect (indicated by +) of ANCCA knockdown on the gene expression in two TNBC cell lines measured by quantitative RT-PCR. See also FIG. 14 for details of the data. B, MDA-MB 468 cells were treated with siRNA targeting ANCCA or control and harvested 48 hrs later for RT-PCR or Western analysis. C. The anti-ANCCA and anti-EZH2 immunoreactivity of the TMA section was scored negative if less than 10% of the epithelial cells displayed any staining, and scored positive if more than 10% of the epithelial cells displayed staining with moderate to high intensity. The association between the different antibody staining was analyzed using Pearson's χ2 test. D. ChIP assays with control and anti-ANCCA antibodies were performed with MDA-MB-468 cells. Co-precipitated genomic DNA was analyzed for ANCCA occupancy at the promoter region of the indicated genes.

FIG. 13 illustrates Unsupervised hierarchical clustering of the 25 probesets (representing 20 genes in addition to ANCCA) exhibiting expression patterns most highly correlated with that of ANCCA overexpression in a microarray data set of 198 patients described in the study by Desmedt C et al. (Clin Cancer Res 2007; 13:3207-14). Spearmann correlation coefficients (i.e., with respect to ANCCA expression) are indicated adjacent to the gene symbols in the heat map. As described in the legend to FIG. 12A, the magnitude of increased or decreased expression (relative to the mean) for each gene across the entire panel of samples is depicted by increasingly darker shades of red or blue, respectively; yellow is indicative of mean expression.

FIG. 14 illustrates ANCCA controls the expression of the top 20 genes with aberrant expression associated with elevated levels of ANCCA in the tumors. Triple-negative breast cancer cells (MDAMB468 and MDA-MB436) are transfected with siRNA targeting ANCCA (black bars) or control RNAi (yellow bars) and harvested 48 hrs later for real-time RT-PCR analysis. Relative transcript levels were obtained by normalization of expression units for each gene with that of GAPDH.

FIG. 15 illustrates ANCCA overexpression correlates strongly with EZH2 in breast cancers. Sections from tissue microarrays were processed for IHC analysis with specific antibodies as indicated. Examples of IHC staining of primary tumors were shown either in low magnification (top panels) or with the areas indicated by dashed frames displayed in high magnification (bottom panels).

FIG. 16 illustrates the functional domains of ANCCA and its involvement in interactions with nuclear receptors and secondary coregulators. The locations of conserved structural and functional domains for the full-length human ANCCA are indicated by filled or textured boxes and bars, with amino acid residues numbered. AD1 and AD2, transcriptional activation domains; AR (androgen receptor); AAA (ATPases associated with various cellular activities); the domains highlighted by the lines or arrow are involved in binding the indicated NR or secondary coactivator. * indicates that the domain's presence or activity is required for coactivation function. References in [ ] are included in the table specifying interactions for each NR-coactivator pair. See, e.g., J. X. Zou, et al., Proc Natl Acad Sci (2007) 104:18067-18072; J. X. Zou, et al., Cancer Research (2009) 69:3339-3346. NTD=coregulator region for interaction with receptor. DBD-hinge=receptor domain for interaction with coregulator.

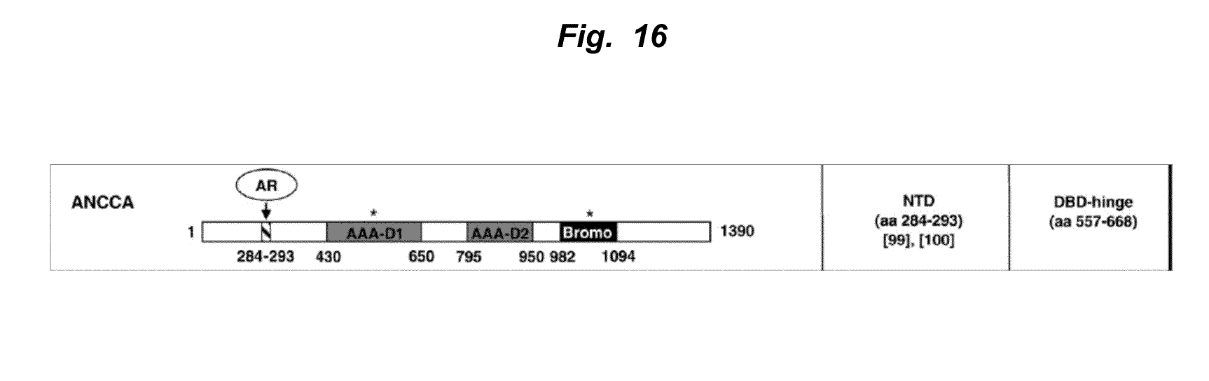

### DETAILED DESCRIPTION

### 1. Introduction

Chromatin coregulators have emerged as important players in tumorigenesis and cancer progression. We previously identified ANCCA as an AAA+ ATPase nuclear coactivator for estrogen receptor-α (ERα) and androgen receptor with crucial function for hormone responsive cancer cell proliferation and the assembly of chromatin modifying complexes. Here, through immunohistochemistry analysis of ANCCA expression in several cohorts of human breast carcinomas (n>300), we show that ANCCA is overexpressed in >70% of breast tumors and that its high level correlates well with tumor histologic grades (P<0.0001) and is a prognostic factor for poor overall survival and disease recurrence. Strikingly, high level ANCCA does not correlate with ERa-positive tumors; instead it associates with ER/progesterone-receptor (PR)/human epidermal growth factor receptor 2 (HER2)-negative diseases. Analysis of ANCCA transcript levels in multiple expression profiling data sets identified ANCCA as a common signature gene and indicates that its elevated transcripts also strongly associate with tumor metastasis and poor survival. Further study found that ANCCA is crucial for proliferation and survival of triple negative/basal-like cancer cells and that it controls the expression of B-Myb, histone methyltransferase EZH2 and an Rb-E2F core program for proliferation, as well as a subset of mitotic kinesins and survival genes (IRS2, VEGF and Aktl). Moreover, overexpression of ANCCA correlates strongly with that of EZH2 in the tumors. These results are consistent with the conclusion that ANCCA acts as a key integrator of multiple oncogenic programs and serves as a prognostic marker and an effective therapeutic target for triple negative cancers.

### 2. Patients Subject to Diagnosis or Treatment

ANCCA is a biomarker located in the cell nucleus, not on the surface of a cell. In the case where the tissue suspected of being cancerous is a solid tissue, expression levels of ANCCA are generally measured on a biopsy tissue sample, e.g., breast tissue. In some embodiments, the biopsy tissue is from a primary tumor.

Accordingly, patients who can benefit from the present method may already present with symptoms of breast cancer. For example, evidence of cancer or a tumor may be present (by visual inspection or palpation, or by scanning techniques, e.g., magnetic resonance imaging (MRI) or Positron Emission Tomography (PET) scans).

The present diagnostic methods find use in conjunction with presently available diagnostic tests for cancer. The patient may already have a preliminary diagnosis of cancer, e.g., based on a serum biomarker or a genetic analysis. Serum biomarkers tests are available for breast cancers, described in, e.g., Lennon, et al., J Clin Oncol. (2009) 27(10):1685-93; and Leary, et al., J Clin Oncol. (2009) 27(10):1694-705. In such cases, a biopsy may be justified and detection of expression levels of ANCCA in the tissue suspected of being cancerous can confirm or contradict a preliminary diagnosis of cancer. In various embodiments, the subject may be suspected of having breast cancer metastasis.

In other cases, the patient may have a personal or familial history of cancer. For example, the patient may be in remission following successful therapeutic treatment of the cancer. The patient may also have tested positive for a gene associated with increased risk of cancer or the recurrence of cancer. In some embodiments, the subject has not or does not respond to tamoxifen therapy. In some embodiments, the subject has an estrogen receptor (ER)-negative breast cancer. In some embodiments, the subject has triple-negative breast cancer. Triple-negative breast cancer is a subtype of breast cancer that is clinically negative for expression of estrogen and progesterone receptors (ER/PR) and HER2 protein.

### 3. Conditions Subject to Diagnosis or Treatment

Conditions subject to a diagnosis, a confirming diagnosis, prognosis, or treatment include any cancer associated with, correlated with or caused by the overexpression of ANCCA, e.g., breast cancer. Usually the cancer will affect a solid tissue, e.g., a carcinoma or a sarcoma, or a tissue subject to solid tumors. Oftentimes, the tissue is an epithelial tissue, e.g., breast carcinoma. In some embodiments, the subject has triple-negative breast cancer. In various embodiments, the subject may be suspected of having breast cancer metastasis.

### 4. Biological Sample

The biological sample from which the expression levels are measured will depend on the tissue suspected on being cancerous. Usually, the biological sample is from the tissue suspected of being cancerous. Usually, the biological sample is from a biopsy.

For example, the biological sample can be from a solid tissue, for example, an epithelial tissue, e.g., breast epithelial tissue. In some embodiments, the biological sample is from a primary tumor in breast tissue. In some embodiments, the biological sample is from a tissue suspected of containing breast cancer metastasis.

In some embodiments, it may be appropriate to measure ANCCA expression levels in a tissue different from the tissue suspected of being cancerous, e.g., when metastasis has occurred.

### 5. Measuring ANCCA Protein Expression

The level of expression of ANCCA can be measured according to methods well known in the art, and described herein. Levels of expression can be measured at the transcriptional and/or protein levels. The level of protein is detected, for example, using directly or indirectly labeled detection agents, e.g., fluorescently, radioactively or enzymatically labeled antibodies. At the protein level, expression of ANCCA can be measured using immunoassays including immunohistochemical staining, Western blotting, ELISA and the like with an antibody that selectively binds to ANCCA or a fragment thereof. Detection of the protein using protein-specific antibodies in immunoassays is known in the art (see, e.g., Harlow & Lane, Using Antibodies: A Laboratory Manual (1998); Coligan, et al., eds., Current Protocols in Immunology (1991-2010); Goding, Monoclonal Antibodies: Principles and Practice (3rd ed. 1996); and Kohler & Milstein, Nature 256:495-497 (1975).

For the purposes of diagnosing the presence of cancer, the expression levels of any isoform of ANCCA protein can be determined. In some embodiments, ANCCA isoform 1 is measured. In some embodiments, ANCCA isoform 2 is measured. Expression levels of ANCCA protein in a tissue suspected of being cancerous can be detected using any method known in the art. Exemplary methods include tissue lysate detection, Western immunoblot and immunohistochemistry, as demonstrated herein.

For detection of the expression levels of ANCCA proteins in a tissue sample, a tissue sample is incubated with an antibody that specifically binds to ANCCA protein under conditions (i.e., time, temperature, concentration of sample) sufficient to allow specific binding. Usually, the tissues are fixed (e.g., in formaldehyde) and permeabilized prior to incubation with antibody, to allow the antibody to access the ANCCA protein in the nucleus. The anti-ANCCA antibodies can be exposed to a tissue sample for about 0.5, 1.0, 1.5, 2.0, 2.5, 3.0 hours, or overnight, about 8, 10 or 12 hours, as appropriate. However, incubation time can be more or less depending on, e.g., the composition of the antigen, the dilution of the sample and the temperature for incubation. Incubations using less diluted samples and higher temperatures can be carried out for shorter periods of time. Incubations are usually carried out at room temperature (about 25° C.) or at biological temperature (about 37° C.), and can be carried out in a refrigerator (about 4° C.). Washing to remove unbound sample before addition of a secondary antibody is carried according to known immunoassay methods.

Labeled secondary antibodies are generally used to detect antibodies or autoantibodies in a sample that have bound to one or more ANCCA polypeptides, antigenic fragments thereof or ANCCA mimeotopes. Secondary antibodies bind to the constant or “C” regions of different classes or isotypes of immunoglobulins—IgM, IgD, IgG, IgA, and IgE. Usually, a secondary antibody against an IgG constant region is used in the present methods. Secondary antibodies against the IgG subclasses, for example, IgG1, IgG2, IgG3, and IgG4, also find use in the present methods. Secondary antibodies can be labeled with any directly or indirectly detectable moiety, including a fluorophore (i.e., fluoroscein, phycoerythrin, quantum dot, Luminex bead, fluorescent bead), an enzyme (i.e., peroxidase, alkaline phosphatase), a radioisotope (i.e., 3H, 32P, 125I) or a chemiluminescent moiety. Labeling signals can be amplified using a complex of biotin and a biotin binding moiety (i.e., avidin, streptavidin, neutravidin). Fluorescently labeled anti-human IgG antibodies are commercially available from Molecular Probes, Eugene, Oreg. Enzyme-labeled anti-human IgG antibodies are commercially available from Sigma-Aldrich, St. Louis, Mo. and Chemicon, Temecula, Calif.

The method of detection of the levels of ANCCA protein in a sample will correspond with the choice of label of the secondary antibody. For example, if tissue lysates containing ANCCA protein is transferred onto a membrane substrate suitable for immunoblotting, the detectable signals (i.e., blots) can be quantified using a digital imager if enzymatic labeling is used or an x-ray film developer if radioisotope labeling is used. Likewise, tissue samples subject to immunohistochemistry can be evaluated using immunofluorescence microscopy or a scanning microscope and automated scanning software capable of detecting and quantifying fluorescent, chemiluminescent, and/or colorimetric signals. Such methods of detection are well known in the art and are described herein.

General immunoassay and immunohistochemical techniques are well known in the art. Guidance for optimization of parameters can be found in, for example, Wu, Quantitative Immunoassay: A Practical Guide for Assay Establishment, Troubleshooting, and Clinical Application, 2000, AACC Press; Principles and Practice of Immunoassay, Price and Newman, eds., 1997, Groves Dictionaries, Inc.; The Immunoassay Handbook, Wild, ed., 2005, Elsevier Science Ltd.; Ghindilis, Pavlov and Atanassov, Immunoassay Methods and Protocols, 2003, Humana Press; Harlow and Lane, Using Antibodies: A Laboratory Manual, 1998, Cold Spring Harbor Laboratory Press; Immunoassay Automation: An Updated Guide to Systems, Chan, ed., 1996, Academic Press; Dabbs, Diagnostic Immunohistochemistry Theranostic and Genomic Applications, 2010, Saunders; Renshaw, Immunohistochemistry: Methods Express Series, 2007, Scion Publishing Ltd.; and Buchwalow and Wicker, Immunohistochemistry: Basics and Methods, 2010, Springer.

The presence or increased presence of ANCCA protein is indicated by a detectable signal (i.e., a blot, fluorescence, chemiluminescence, color, radioactivity) in an immunoassay or immunohistochemical assay, where the biological sample from the patient is contacted with antibody or antibody fragment that specifically binds to ANCCA protein. This detectable signal can be compared to the signal from a normal or non-cancerous control sample or to a threshold value. In some embodiments, increased expression levels of ANCCA protein are detected, and the presence or increased risk of breast cancer, progression of breast cancer or breast cancer metastasis is indicated, e.g., when the detectable signal of ANCCA protein expression levels in the test sample is at least about 10%, 20%, 30%, 50%, 75% greater in comparison to the signal of ANCCA protein in the normal or non-cancerous control sample or the predetermined threshold value. In some embodiments, an increased expression levels of ANCCA protein is detected, and the presence or an increased risk of breast cancer, progression of breast cancer or breast cancer metastasis is indicated, when the detectable signal of ANCCA protein in the test sample is at least about 1-fold, 2-fold, 3-fold, 4 fold or more, greater in comparison to the signal of ANCCA protein in the normal or non-cancerous control sample or the predetermined threshold value. Usually, the sample and control or predetermined threshold levels are from the same tissue types.

In some embodiments, the ANCCA protein levels are compared with an ANCCA protein expression level control from tissue known to be cancerous. In this case, ANCCA protein expression levels in the test biological sample equivalent to or greater than the positive control sample, known to be cancerous, are indicative of breast cancer, progression of breast cancer or breast cancer metastasis. Usually, the sample and control or predetermined threshold levels are from the same tissue types.

Alternatively, if the ANCCA protein expression levels in the test biological sample are less than the ANCCA protein expression levels in the positive cancerous tissue control or the predetermined threshold level, then a diagnosis of breast cancer, progression of breast cancer or breast cancer metastasis is generally not indicated. Likewise, if the ANCCA protein expression levels in the test biological sample are equivalent to or less than a normal or non-cancerous control or the predetermined threshold level, then a diagnosis of breast cancer, progression of breast cancer or breast cancer metastasis is not indicated.

In some embodiments, the results of the ANCCA protein expression level determinations are recorded in a tangible medium. For example, the results of the present diagnostic assays (e.g., the observation of the presence or increased presence of ANCCA protein) and the diagnosis of whether or not the presence or an increased risk of cancer is determined can be recorded, e.g., on paper or on electronic media (e.g., audio tape, a computer disk, a CD, a flash drive, etc.).

In some embodiments, the methods further comprise the step of providing the diagnosis to the patient of whether or not there is the presence or an increased risk of cancer in the patient, e.g., based on the results of the ANCCA protein expression level determinations.

### 6. Measuring ANCCA Transcription Expression

ANCCA expression levels can also be detected at the transcriptional level, using any method known in the art. At the transcriptional level, mRNA can be detected by, for example, amplification, e.g., RT-PCR, PCR, LCR, or hybridization assays, e.g., northern hybridization, RNAse protection, or dot blotting, of mRNA in tissue lysate or in situ in tissue sections, all methods known in the art. The level of mRNA is detected, for example, using directly or indirectly labeled detection agents, e.g., fluorescently or radioactively labeled nucleic acids. These assays are well-known to those of skill in the art and described in, e.g., Ausubel, et al., eds., Current Protocols In Molecular Biology (1987-2010); PRINS and In Situ PCR Protocols (Methods in Molecular Biology), Pellestor, eds, 2006, Human Press; Bagasra and Hansen, In-Situ PCR Techniques, 1997, Wiley-Liss; PCR 3: PCR In Situ Hybridization: A Practical Approach (Vol 3), Herrington and O'Leary, eds., 1998, Oxford University Press; Nuovo, PCR In Situ Hybridization: Protocols and Applications, 1997, Lippincott Williams & Wilkins; In Situ Hybridization Protocols (Methods in Molecular Biology), Darby and Hewitson, eds., 2005, Human Press; Fluorescence In Situ Hybridization (FISH)— Application Guide, Liehr, ed., 2009, Springer Berlin Heidelberg, Schwarzacher and Heslop-Harrison, Practical in Situ Hybridization, 2000, BIOS Scientific Publishers.

Polynucleotides that specifically bind to an expressed ANCCA nucleic acid sequence can be labeled with any directly or indirectly detectable moiety, including a fluorophore (i.e., fluoroscein, phycoerythrin, quantum dot, Luminex bead, fluorescent bead), an enzyme (i.e., peroxidase, alkaline phosphatase), a radioisotope (i.e. ³H, ³²P, ¹²⁵I) or a chemiluminescent moiety. Labeling signals can be amplified using a complex of biotin and a biotin binding moiety (i.e., avidin, streptavidin, neutravidin).

The presence or increased presence of ANCCA mRNA is indicated by a detectable signal (i.e., a blot, fluorescence, chemiluminescence, color, radioactivity) in an ANCCA nucleic acid amplification assay, where the biological sample from the patient (e.g., tissue lysate or tissue section) is contacted with a labeled polynucleotide that specifically hybridizes to an ANCCA nucleic acid sequence. This detectable signal can be compared to the signal from a normal or non-cancerous control sample or to a threshold value. In some embodiments, increased expression levels of ANCCA mRNA are detected, and the presence or increased risk of breast cancer, progression of breast cancer or breast cancer metastasis is indicated, e.g., when the detectable signal of ANCCA mRNA expression levels in the test sample is at least about 10%, 20%, 30%, 50%, 75% greater in comparison to the signal of ANCCA mRNA in the normal or non-cancerous control sample or the predetermined threshold value. In some embodiments, an increased expression levels of ANCCA mRNA is detected, and the presence or an increased risk of breast cancer, progression of breast cancer or breast cancer metastasis is indicated, when the detectable signal of ANCCA mRNA in the test sample is at least about 1-fold, 2-fold, 3-fold, 4 fold or more, greater in comparison to the signal of ANCCA mRNA in the normal or non-cancerous control sample or the predetermined threshold value. Usually, the sample and control or predetermined threshold levels are from the same tissue types.

In some embodiments, the ANCCA mRNA levels are compared with an ANCCA mRNA expression level control from tissue known to be cancerous. In this case, ANCCA mRNA expression levels in the test biological sample equivalent to or greater than the positive control sample, known to be cancerous, are indicative of breast cancer, progression of breast cancer or breast cancer metastasis. Usually, the sample and control or predetermined threshold levels are from the same tissue types.

Alternatively, if the ANCCA mRNA expression levels in the test biological sample are less than the ANCCA mRNA expression levels in the positive cancerous tissue control or the predetermined threshold level, then a diagnosis of breast cancer, progression of breast cancer or breast cancer metastasis is generally not indicated. Likewise, if the ANCCA mRNA expression levels in the test biological sample are equivalent to or less than a normal or non-cancerous control or the predetermined threshold level, then a diagnosis of breast cancer, progression of breast cancer or breast cancer metastasis is not indicated.

In some embodiments, the results of the ANCCA mRNA expression level determinations are recorded in a tangible medium. For example, the results of the present diagnostic assays (e.g., the observation of the presence or increased presence of ANCCA mRNA) and the diagnosis of whether or not the presence or an increased risk of breast cancer, progression of breast cancer and/or breast cancer metastasis is determined can be recorded, e.g., on paper or on electronic media (e.g., audio tape, a computer disk, a CD, a flash drive, etc.).

In some embodiments, the methods further comprise the step of providing the diagnosis to the patient of whether or not there is the presence or an increased risk of breast cancer, progression of breast cancer and/or breast cancer metastasis in the patient, e.g., based on the results of the ANCCA mRNA expression level determinations.

### 7. Monitoring Efficacy of Anticancer Treatment

The differentially expressed or overexpressed ANCCA correlated with the presence of cancer also allows for the course of treatment of a cancer associated with or caused by overexpression of ANCCA to be monitored (e.g., breast cancer). In this method, a test cell population from a biological sample is provided from a subject undergoing treatment for a cancer associated with or caused by overexpression of ANCCA. As appropriate, test cell populations from biological samples are obtained from the subject at various time points, e.g., before, during, and/or after a course of treatment. Expression of ANCCA in the test cell population is then determined and compared to a reference cell population which includes cells whose cancer state and ANCCA expression level is known. In the context of the present invention, the reference cell population has not been exposed to the treatment of interest.

If the reference cell population contains no cancer cells or is representative of normal tissue, a similarity in the expression levels of ANCCA in the test cell population and the reference cell population indicates that the treatment of interest is efficacious (i.e., increased predicted survival). However, an increase in the expression levels of ANCCA in the test cell population in comparison to a normal control reference cell population indicates a less favorable clinical outcome or prognosis (e.g., decreased predicted survival). In this case, the threshold level of ANCCA expression is represented by the level ANCCA expression in a non-cancerous cell population. Similarly, if the reference cell population contains cancer cells, a decrease in expression levels of ANCCA in the test cell population in comparison to the reference cell population indicates that the treatment of interest is efficacious, while a similarity in the expression of ANCCA in the test population and an affirmed cancer control reference cell population indicates a less favorable clinical outcome or prognosis. In this case, the threshold level of ANCCA expression is represented by the level ANCCA expression in a cancerous cell population.

Additionally, the expression level of ANCCA determined in a biological sample from a subject obtained after treatment (i.e., post-treatment levels) can be compared to the expression level of ANCCA determined in a biological sample from a subject obtained prior to treatment onset (i.e., pre-treatment levels). A decrease in the expression level of ANCCA in a post-treatment sample indicates that the treatment of interest is efficacious while an increase or maintenance in the expression level in the post-treatment sample indicates a less favorable clinical outcome or prognosis. In this case, the threshold level of ANCCA expression is represented by the level ANCCA expression in an untreated cell population, e.g., from the same subject receiving a treatment or preventative regime.

As used herein, the term “efficacious” indicates that the treatment leads to a reduction in the expression of a pathologically up-regulated gene, or a decrease in size, prevalence, or metastatic potential of cancer in a subject. When a treatment of interest is applied prophylactically, the term “efficacious” means that the treatment retards or prevents cancer from forming or retards, prevents, or alleviates a symptom of cancer (e.g., to prevent or inhibit metastasis or to prevent or inhibit the recurrence of breast cancer in a subject in remission). Assessment of cancer or tumors can be made using standard clinical protocols. In addition, efficaciousness can be determined in association with any known method for diagnosing or treating cancer. Cancer can be diagnosed, for example, by identifying symptomatic anomalies, e.g., weight loss, abdominal pain, back pain, anorexia, nausea, vomiting and generalized malaise, weakness, presence of tumor and jaundice.

### 8. Methods of Inhibiting the Activity of ANCCA

Subjects determined to have a breast cancer that overexpresses ANCCA can be administered an appropriate therapeutic or preventative regime that inhibits the activity of ANCCA, at either or both the protein and/or transcriptional level. Accordingly, the invention further provides methods of reducing, inhibiting or preventing the growth of a breast cancer cell or a tumor the overexpresses ANCCA by contacting the cancer cell or tumor that overexpresses ANCCA with an agent that inhibits ANCCA, at either or both the protein and/or transcriptional level.

a. Inhibiting the Activity of ANCCA at the Protein Level

In various embodiments, the activity of ANCCA is inhibited by inhibiting the activity of ANCCA at the protein level. ANCCA is a member of the AAA+ (ATPases associated with various cellular activities) family, and is a transcriptional coactivator for estrogen receptor alpha (ERalpha) and androgen receptor (AR) as well as c-Myc [J. X. Zou, et al., Proc Natl Acad Sci (2007) 104:18067-18072; J. X. Zou, et al., Cancer Research (2009) 69:3339-3346; and M. Ciro, et al., Cancer Research (2009) 69:8491-8498.]. Members of this functionally diverse ATPase family possess conserved AAA+ ATP-binding domains and assemble into characteristic ring-shaped hexamers constituting the active ATPase holoenzyme. ATP binding sites are formed at the interface between adjacent AAA+ protein subunits. The AAA+ domain contains several structural motifs, particularly within the key SRH (second region of homology) element, believed to coordinate ATP hydrolysis and the propagation of conformational changes throughout the enzyme assembly. The dynamic coupling of these two events then drives conformational changes in substrate proteins during cellular processes that involve protein unfolding and protein-complex remodeling, such as proteolysis, membrane fusion, DNA replication, and microtubule sliding [P. I. Hanson, et al., Nature Reviews Molecular Cell Biology (2005) 6:519-529; C. Indiani, et al., Nature Reviews Molecular Cell Biology 7 (2006) 751-761; J. P. Erzberger, et al., Annual Review of Biophysics and Biomolecular Structure (2006) 35:93-114; and S. A. Burgess, et al., Nature (2003) 421:715-718]. Based on structure-function analysis of several AAA+ proteins, residues extending from each subunit into the central pore formed by the hexamer are involved in substrate binding and translocation through the pore during processing [S. M. Siddiqui, et al., Genes & Development (2004) 18:369-374; C. Schlieker, et al., Nature Structural and Molecular Biology (2004) 11:607-615; A. Martin, et al., Nature Structural and Molecular Biology (2008) 15:1147-1151].

The predicted structure of ANCCA includes a bromodomain located C-terminal to two centrally situated AAA+ domains that are most closely related in sequence homology to the so-called classic Glade of AAA+ proteins such as p97/VCP [J. X. Zou, et al., Proc Natl Acad Sci USA (2007) 104:18067-18072; J. X. Zou, et al., Cancer Research (2009) 69:3339-3346]. ANCCA binds and hydrolyzes ATP [J. X. Zou, et al., Proc Natl Acad Sci USA (2007) 104:18067-18072] and data suggests that ANCCA forms multimers. Interestingly, mutating key residues of the Walker A and Walker B motifs in the first AAA+domain disrupt ANCCA function as an ER coactivator, indicating the involvement of ATPase activity [J. X. Zou, et al., Proc Natl Acad Sci USA (2007) 104:18067-18072]. Integrity of the bromodomain, which is often found in both HAT containing and ATP-dependent chromatin remodeling proteins [R. Marmorstein, et al., Gene (2001) 272:1-9], also affects the ability of ANCCA to function as a coactivator (Revenko A. et. al., Mol Cell Biol. (2010) 30(22):5260-72). Based on other AAA+ proteins, mutation of a key arginine residue in the conserved SRH element impairs oligomerization and ATPase activity [T. Ogura, et al., Journal of Structural Biology 146 (2004) 106-112]. This approach may be appropriate to determine whether ANCCA multimer assembly is required for its coactivator function. Alternatively, structural determinants responsible for ANCCA-nuclear hormone receptor (NR) interaction could be identified and targeted for specific disruption of ANCCA-mediated coactivation. For example, ANCCA directly associates with the DBD-hinge region of AR primarily through an N-terminal region that lies outside the first AAA+domain [J. X. Zou, et al., Cancer Research (2009) 69:3339-3346].

Generally, the ATPase activity of an ANCCA enzyme, e.g., having at least 90% sequence identity, e.g., at least about 91%, 92%, 93%, 94%, 95%, 96%, 97%, 98% or 99% sequence identity, to an amino acid sequence of SEQ ID NO:49 is inhibited or reduced, thereby preventing, reducing, delaying or inhibiting breast cancer, the progression of breast cancer and/or breast cancer metastasis. In some embodiments, the ANCCA enzyme to be inhibited has an amino acid sequence of SEQ ID NO:49. In various embodiments, the ANCCA enzyme to be inhibited is human.

In one embodiment, the methods involve reducing, inhibiting or preventing breast cancer, progression of breast cancer and/or breast cancer metastasis by inhibiting the activity of ANCCA at the protein level (e.g., substrate binding, ATPase activity, multimer formation, nuclear receptor binding). Preferably, the inhibitory agent specifically or preferentially inhibits the protein activity of ANCCA.

Examples of agents capable of inhibiting enzyme activity include substrate analogs, suicide substrates, alkylating agents, and inhibitory nucleic acids (reviewed in Ferscht, Structure and Mechanism in Protein Science: A Guide to Enzyme Catalysis and Protein Folding, 3rd Edition, 1999, W.H. Freeman & Co.). The methods of decreasing, inhibiting or preventing ANCCA activity can involve administering to a subject, including a mammal such as a human, a compound that is an analog of a substrate (e.g., analog of ATP), including small organic compound or peptidomimetic substrate analogs.

The preferred ANCCA inhibitors have no other adverse effects on cellular metabolism, so that other cellular functions proceed while the specific reaction of ANCCA ATPase activity is inhibited. The blocking agents are preferably relatively small molecules, thereby avoiding immunogenicity and allowing passage through the cell membrane. Ideally, they have a molecular weight of between about 100-2000 daltons, but may have molecular weights up to 5000 or more, depending upon the desired application. In most preferred embodiments, the inhibitors have molecular weights of between about 200-600 daltons.

The inhibitors of the present invention preferably have strong affinity for the target enzyme, so that at least about 60-70% inhibition of ANCCA activity is achieved, more preferably about 75%-85% and most preferably 90%-95% or more. In some embodiments, the inhibitors will completely inhibit ANCCA activity. The affinity of the enzyme for the inhibitor is preferably sufficiently strong that the dissociation constant, or Kᵢ, of the enzyme-inhibitor complex is less than about 10⁻⁵ M, typically between about 10⁻⁶ and 10⁻⁸ M.

Enzyme inhibition generally involves the interaction of a substance with an enzyme so as to decrease the rate of the reaction catalyzed by that enzyme Inhibitors can be classified according a number of criteria. For example, they may be reversible or irreversible. An irreversible inhibitor dissociates very slowly, if at all, from its target enzyme because it becomes very tightly bound to the enzyme, either covalently or noncovalently. Reversible inhibition, in contrast, involves an enzyme-inhibitor complex which may dissociate.

Inhibitors can also be classified according to whether they are competitive, noncompetitive or uncompetitive inhibitors. In competitive inhibition for kinetically simple systems involving a single substrate, the enzyme can bind either the substrate or the inhibitor, but not both. Typically, competitive inhibitors resemble the substrate or the product(s) and bind the active site of the enzyme, thus blocking the substrate from binding the active site. A competitive inhibitor diminishes the rate of catalysis by effectively reducing the affinity of the substrate for the enzyme. Typically, an enzyme may be competitively inhibited by its own product because of equilibrium considerations. Since the enzyme is a catalyst, it is in principle capable of accelerating a reaction in the forward or reverse direction.

Noncompetitive inhibitors allow the enzyme to bind the substrate at the same time it binds the inhibitor. A noncompetitive inhibitor acts by decreasing the turnover number of an enzyme rather than diminishing the proportion of free enzyme. Another possible category of inhibition is mixed or uncompetitive inhibition, in which the inhibitor affects the binding site and also alters the turnover number of the enzyme. Enzyme inhibition of kinetically complex systems involving more than one substrate, as can be the case for ANCCA enzymes, are described in Segel, Enzyme Kinetics, (Wiley, N. Y. 1975).

ANCCA ATPase activity and its inhibition or enhancement is typically assayed according to standard methods for determining enzyme activity. For a general discussion of enzyme assays, see, Rossomando, “Measurement of Enzyme Activity” in Guide to Protein Purification, Vol. 182, Methods in Enzymology (Deutscher ed., 1990). When testing for the ability of a test compound to decrease or inhibit ANCCA ATPase activity, the ATPase activity of an ANCCA enzyme exposed to the test compound is compared to the ATPase activity of an ANCCA enzyme in a control unexposed to the test compound.

1. Anti-ANCCA Antigen Binding Molecule Inhibitors

Antigen binding molecules that bind to and reduce or inhibit the protein activities (e.g., substrate and/or receptor binding, multimerization and/or ATPase activities) of ANCCA can be non-antibody binding molecules, or antibodies and fragments thereof. The antigen binding molecules can bind to any region of the enzyme that inhibits or reduces its protein activity, e.g., by interfering directly with catalytic activity, multimerization, receptor binding and/or substrate binding. In various embodiments, the antigen binding molecule binds to the AAA+ domain, the ATP hydrolysis site and/or the bromodomain.

a. Non-Antibody Antigen Binding Molecules

In various embodiments, the antigen binding molecule is a non-antibody binding protein. Protein molecules have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc. Natl. Acad. Sci. U.S.A. 92(14):6552-6556 (1995)) discloses an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7, 1 15,396) discloses an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc. Natl. Acad. Sci. U.S.A. 96(5): 1898-1903 (1999)) discloses an antibody mimic based on a lipocalin scaffold (Anticalin®). Lipocalins are composed of a β-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies. Anticalins® are small, single chain peptides, typically between 160 and 180 residues, which provide several advantages over antibodies, including decreased cost of production, increased stability in storage and decreased immunological reaction.

Hamilton et al. (U.S. Pat. No. 5,770,380) discloses a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell. MoI. Biol. 49(2):209-216 (2003)) discusses a methodology for reducing antibodies into smaller peptidomimetics, they term “antibody like binding peptidomimetics” (ABiP) which can also be useful as an alternative to antibodies.

Silverman et al. (Nat. Biotechnol. (2005), 23: 1556-1561) discloses fusion proteins that are single-chain polypeptides comprising multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can comprise multiple independent binding domains that can exhibit improved affinity (in some cases sub-nanomolar) and specificity compared with single-epitope binding proteins. Additional details concerning methods of construction and use of avimers are disclosed, for example, in U.S. Patent App. Pub. Nos. 20040175756, 20050048512, 20050053973, 20050089932 and 20050221384.

In addition to non-immunoglobulin protein frameworks, antibody properties have also been mimicked in compounds comprising RNA molecules and unnatural oligomers (e.g., protease inhibitors, benzodiazepines, purine derivatives and beta-turn mimics) all of which are suitable for use with the present invention.

b. Anti-ANCCA Antibodies

In various embodiments, the antigen binding molecule is an antibody or antibody fragment that binds to ANCCA and inhibits the protein activity of ANCCA (e.g., nuclear receptor binding, substrate binding, ATPase activity, multimerization). Such anti-ANCCA antibodies are useful for preventing, delaying, inhibiting and treating breast cancer, progression of breast cancer, and/or breast cancer metastasis.

An antibody suitable for treating, mitigating, delaying and/or preventing breast cancer, progression of breast cancer, and/or breast cancer metastasis in a subject is specific for at least one portion of the ANCCA polypeptide, e.g., the catalytic domain, the AAA+ domain, transcriptional activation domain and/or the bromodomain. For example, one of skill in the art can use peptides derived from the targeted ANCCA domain of an ANCCA polypeptide to generate appropriate antibodies suitable for use with the invention. Illustrative, non-limiting amino sequences suitable for use in selecting peptides for use as antigens include an amino acid of SEQ ID NO:49, and fragments thereof including the targeted domain.

Anti-ANCCA antibodies for use in the present methods include without limitation, polyclonal antibodies, monoclonal antibodies, chimeric antibodies, humanized antibodies, human antibodies, and fragments thereof.

The preparation of polyclonal antibodies is well-known to those skilled in the art. See, for example, Green et al., Production of Polyclonal Antisera, in IMMUNOCHEMICAL PROTOCOLS (Manson, ed), pages 1-5 (Humana Press 1992), Coligan et al, Production of Polyclonal Antisera in Rabbits, Rats. Mice and Hamsters, in CURRENT PROTOCOLS IN IMMUNOLOGY, section 2 4 1 (1992), which are hereby incorporated by reference.

The preparation of monoclonal antibodies likewise is conventional. See, for example, Kohler & Milstem, Nature 256 495 (1975). Coligan et al., sections 2.5.1-2.6.7, Harlow et al, ANTIBODIES A LABORATORY MANUAL, page 726 (Cold Spring Harbor Pub 1988, and Harlow, USING ANTIBODIES A LABORATORY MANUAL, Cold Spring Harbor Laboratory Press, 1998), which are hereby incorporated by reference Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al. sections 2.7.1-2.7.12 and sections 2.9.1-2.9.3, Barnes et al., Purification of Immunoglobulin G (IgG), in METHODS IN MOLECULAR BIOLOGY, VOL 10, pages 79-104 (Humana Press 1992).

Methods of in vitro and in vivo multiplication of monoclonal antibodies is well-known to those skilled in the art Multiplication in vitro can be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo can be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Anti-ANCCA antibodies can be altered or produced for therapeutic applications. For example, antibodies of the present invention can also be derived from subhuman primate antibody. General techniques for raising therapeutically useful antibodies in baboons can be found, for example, in Goldenberg et al., International Patent Publication WO 91/11465 (1991) and Losman et al., Int. J. Cancer 46:310 (1990), which are hereby incorporated by reference.

Alternatively, therapeutically useful anti-ANCCA antibodies can be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi, et al., Proc. Nat'l Acad. Sci. USA 86:3833 (1989), which is hereby incorporated in its entirety by reference. Techniques for producing humanized monoclonal antibodies are described, for example, by Jones et al., Nature 321:522 (1986); Riechmann et al., Nature 332:323 (1988); Verhoeyen et al., Science 239:1534 (1988); Carter et al. Proc. Nat'l Acad. Sci. USA 89:4285 (1992); Sandhu, Crit. Rev. Biotech. 12:437 (1992); and Singer et al., J. Immunol. 150:2844 (1993), which are hereby incorporated by reference.

Anti-ANCCA antibodies for use in the present methods also can be derived from human antibody fragments isolated from a combinatorial immunoglobulin library. See, for example, Barbas, et al., METHODS: A COMPANION TO METHODS IN ENZYMOLOGY, VOL. 2, page 119 (1991); Winter et al., Ann. Rev. Immunol. 12:433 (1994), which are hereby incorporated herein by reference. Cloning and expression vectors that are useful for producing a human immunoglobulin phage library can be obtained, for example, from STRATAGENE Cloning Systems (now Agilent Technologies).

In addition, anti-ANCCA antibodies for the mitigation, delay, treatment and/or prevention of breast cancer, progression of breast cancer, and/or breast cancer metastasis can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al. Nature 368:856 (1994); and Taylor et al., Int. Immunol. 6:579 (1994), which are hereby incorporated by reference.

In various embodiments, the antibodies are human IgG immunoglobulin. As appropriate or desired, the IgG can be of an isotype to promote antibody-dependent cell-mediated cytotoxicity (ADCC) and/or complement-dependent cellular cytotoxicity (CDCC), e.g., human IgG1 or human IgG3.

Antibody fragments for use in the present methods can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a fragment denoted F(ab′)₂— This fragment can be further cleaved using a thiol reducing agent, and optionally a blocking group for the sulfhydryl groups resulting from cleavage of disulfide linkages, to produce Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein. These patents are hereby incorporated in their entireties by reference. See also, Nisonhoff, et al., Arch. Biochem. Biophys. 89:230 (1960); Porter, Biochem. J. 73:119 (1959): Edelman et al., METHODS IN ENZYMOLOG Y, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques can also be used, so long as the fragments bind to the antigen that is recognized by the intact antibody.

For example, Fv fragments comprise an association of VH and VL chains. This association can be noncovalent, as described in Inbar et al., Proc. Nat'l Acad. Sci.

USA 69:2659 (1972). Alternatively, the variable chains can be linked by an intermolecular disulfide bond or cross-linked by chemicals such as glutaraldehyde. See. e. g. Sandhu, Crit Rev Biotechnol. 1992; 12(5-6):437-62. In some embodiments, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFv are described, for example, by Whitlow et al, METHODS: A COMPANION TO METHODS IN ENZYMOLOGY, VOL. 2, page 97 (1991); Bird et al, Science 242:423-426 (1988); Ladner, et al, U.S. Pat. No. 4,946,778; Pack, et al, BioTechnology 11:1271 77 (1993); and Sandhu, supra.

Another form of an antibody fragment suitable for use with the methods of the present invention is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al, METHODS: A COMPANION TO METHODS IN ENZYMOLOG Y, VOL. 2, page 106 (1991), iv. Small Organic Compounds.

In some embodiments, the anti-ANCCA antibody is a single-domain antibody (sdAb) or a nanobody. A single-domain antibody or a nanobody is a fully functional antibody that lacks light chains; they are heavy-chain antibodies containing a single variable domain (VHH) and two constant domains (CH2 and CH3). Like a whole antibody, single domain antibodies or nanobodies are able to bind selectively to a specific antigen. With a molecular weight of only 12-15 kDa, single-domain antibodies are much smaller than common antibodies (150-160 kDa) composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). Nanobodies are more potent and more stable than conventional four-chain antibodies which leads to (1) lower dosage forms, less frequent dosage leading to less side effects; and (2) improved stability leading to a broader choice of administration routes, comprising oral or subcutaneous routes and slow-release formulations in addition to the intravenous route. Slow-release formulation with stable anti-ANCCA nanobodies, find use for the mitigation, delay, treatment and prevention of breast cancer, progression of breast cancer, and/or breast cancer metastasis, avoiding the need of repeated injections and the side effects associated with it. Because of their small size, nanobodies have the ability to cross membranes and penetrate into physiological compartments, tissues and organs not accessible to other, larger polypeptides and proteins.

Preferably, the antibodies are humanized for use in treating or preventing breast cancer, progression of breast cancer, and/or breast cancer metastasis in humans.

2. Screening for Inhibitors of ANCCA

One can identify lead compounds that are suitable for further testing to identify those that are therapeutically effective inhibitory agents by screening a variety of compounds and mixtures of compounds for their ability to decrease or inhibit ANCCA protein activity (e.g., nuclear receptor binding, substrate binding, ATPase activity and/or multimerization) in in vivo and in vitro assays, as described herein and known in the art.

The use of screening assays to discover naturally occurring compounds with desired activities is well known and has been widely used for many years. For instance, many compounds with antibiotic activity were originally identified using this approach. Examples of such compounds include monolactams and aminoglycoside antibiotics. Compounds which inhibit various enzyme activities have also been found by this technique, for example, mevinolin, lovastatin, and mevacor, which are inhibitors of hydroxymethylglutamyl Coenzyme A reductase, an enzyme involved in cholesterol synthesis. Antibiotics that inhibit glycosyltransferase activities, such as tunicamycin and streptovirudin have also been identified in this manner.

Thus, another important aspect of the present invention is directed to methods for screening samples for inhibition or reduction of ANCCA protein activity. A “sample” as used herein can be any mixture of compounds suitable for testing in an ANCCA ATPase or binding or multimerization assay. A typical sample comprises a mixture of synthetically produced compounds or alternatively a naturally occurring mixture, such as a cell culture broth. Suitable cells include any cultured cells such as mammalian, insect, microbial or plant cells. Microbial cell cultures are composed of any microscopic organism such as bacteria, protozoa, yeast, fungi and the like.

In the typical screening assay, an isolated and/or purified ANCCA protein or a biological sample (e.g., cancer cells that overexpress ANCCA), is added to a standard ANCCA enzymatic and/or binding assay. If inhibition or enhancement of activity as compared to control assays is found, the mixture is usually fractionated to identify components of the sample providing the inhibiting or enhancing activity. The sample is fractionated using standard methods such as ion exchange chromatography, affinity chromatography, electrophoresis, ultrafiltration, HPLC and the like. See, e.g., Scopes, Protein Purification, Principles and Practice, 3rd Edition, 1994, Springer-Verlag. Each isolated fraction is then tested for inhibiting or enhancing activity. If desired, the fractions are then further sub fractionated and tested. This sub fractionation and testing procedure can be repeated as many times as desired.

By combining various standard purification methods, a substantially pure compound suitable for in vivo therapeutic testing can be obtained. A substantially pure modulating agent as defined herein is an activity inhibiting or enhancing compound which migrates largely as a single band under standard electrophoretic conditions or largely as a single peak when monitored on a chromatographic column. More specifically, compositions of substantially pure modulating agents will comprise less than ten percent miscellaneous compounds.

In preferred embodiments, the assays are designed to screen large chemical libraries by automating the assay steps and providing compounds from any convenient source to assays, which are typically run in parallel (e.g., in microtiter formats on microtiter plates in robotic assays).

As noted, the invention provides in vitro assays for ANCCA activity in a high throughput format. For each of the assay formats described, “no inhibitor” control reactions which do not include an inhibitory agent provide a background level of ANCCA activity. In the high throughput assays of the invention, it is possible to screen up to several thousand different modulators in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many different plates per day; assay screens for up to about 6,000-20,000, and even up to about 100,000-1,000,000 different compounds is possible using the integrated systems of the invention. The steps of labeling, addition of reagents, fluid changes, and detection are compatible with full automation, for instance using programmable robotic systems or “integrated systems” commercially available, for example, through BioTX Automation, Conroe, Tex.; Qiagen, Valencia, Calif.; Beckman Coulter, Fullerton, Calif.; and Caliper Life Sciences, Hopkinton, Mass.

In some assays it will be desirable to have positive controls to ensure that the components of the assays are working properly. For example, a known inhibitor of ANCCA activity can be incubated with one sample of the assay, and the resulting increase or decrease in signal determined according to the methods herein.

Essentially any chemical compound can be screened as a potential inhibitor of ANCCA activity in the assays of the invention. Most preferred are generally compounds that can be dissolved in aqueous or organic (especially DMSO-based) solutions and compounds which fall within Lipinski's “Rule of 5” criteria. The assays are designed to screen large chemical libraries by automating the assay steps and providing compounds from any convenient source to assays, which are typically run in parallel (e.g., in microtiter formats on multiwell plates in robotic assays). It will be appreciated that there are many suppliers of chemical compounds, including Sigma-Aldrich (St. Louis, Mo.); Fluka Chemika-Biochemica Analytika (Buchs Switzerland), as well as numerous providers of small organic molecule libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), Tripos, Inc. (St. Louis, Mo.), Reaction Biology Corp. (Malvern, Pa.), Biomol Intl. (Plymouth Meeting, Pa.), TimTec (Newark, Del.), and AnalytiCon (Potsdam, Germany), among others.

In one preferred embodiment, inhibitors of ANCCA activity are identified by screening a combinatorial library containing a large number of potential therapeutic compounds (potential inhibitor compounds). Such “combinatorial chemical or peptide libraries” can be screened in one or more assays, as described herein, to identify those library members particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with .beta.-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.).

Lead compounds that have been identified for their capability to reduce or inhibit ANCCA activity in vitro are then evaluated for their ability to prevent, reduce or inhibit the ability to prevent, reduce or inhibit growth or proliferation of breast cancer cells in vitro and in in vivo animal models, known in the art.

b. Inhibiting the Expression of ANCCA

In various embodiments, the activity of ANCCA is inhibited by inhibiting the expression of ANCCA, e.g., at the transcriptional level. Accordingly, the invention further provides methods of reducing, inhibiting or preventing the growth of a breast cancer cell or a tumor the overexpresses ANCCA by contacting the cancer cell or tumor that overexpresses ANCCA with an inhibitory nucleic acid (e.g., antisense RNA, ribozyme, short inhibitory RNA, micro RNA, etc.) that selectively hybridizes to an ANCCA nucleic acid sequence. The cancer cell or tumor can be contacted in vitro or in vivo.

Relatedly, the invention further provides reducing or inhibiting the expression of an ANCCA nucleic acid in a cancer cell by contacting the cancer cell with an ANCCA inhibitory nucleic acid (e.g., a ribozyme, an antisense RNA, a short-inhibitory RNA (siRNA), a micro RNA (miRNA)). The cancer cell may be in vitro or in vivo.

A nucleic acid molecule complementary to at least a portion of the ANCCA gene can be used to inhibit ANCCA gene expression. Means for inhibiting gene expression using short RNA molecules, for example, are known. Among these are short interfering RNA (siRNA), small temporal RNAs (stRNAs), and micro-RNAs (miRNAs). Short interfering RNAs silence genes through a mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8(6):842-50 (2002); Morris et al., Science, 305(5688):1289-92 (2004); He and Hannon, Nat Rev Genet. 5(7):522-31 (2004).

“RNA interference,” a form of post-transcriptional gene silencing (“PTGS”), describes effects that result from the introduction of double-stranded RNA into cells (reviewed in Fire, A. Trends Genet. 15:358-363 (1999); Sharp, P. Genes Dev 13:139-141 (1999); Hunter, C. Curr Biol 9:R440-R442 (1999); Baulcombe. D. Curr Biol 9:R599-R601 (1999); Vaucheret et al. Plant J 16: 651-659 (1998)). RNA interference, commonly referred to as RNAi, offers a way of specifically inactivating a cloned gene, and is a powerful tool for investigating gene function.

The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. While RNAi was shown initially to work well in lower eukaryotes, for mammalian cells, it was thought that RNAi might be suitable only for studies on the oocyte and the preimplantation embryo.

In mammalian cells other than these, however, longer RNA duplexes provoked a response known as “sequence non-specific RNA interference,” characterized by the non-specific inhibition of protein synthesis.

Further studies showed this effect to be induced by dsRNA of greater than about 30 base pairs, apparently due to an interferon response. It is thought that dsRNA of greater than about 30 base pairs binds and activates the protein PKR and 2′,5′-oligonucleotide synthetase (2′,5′-AS). Activated PKR stalls translation by phosphorylation of the translation initiation factors eIF2α, and activated 2′,5′-AS causes mRNA degradation by 2′,5′-oligonucleotide-activated ribonuclease L. These responses are intrinsically sequence-nonspecific to the inducing dsRNA; they also frequently result in apoptosis, or cell death. Thus, most somatic mammalian cells undergo apoptosis when exposed to the concentrations of dsRNA that induce RNAi in lower eukaryotic cells.

More recently, it was shown that RNAi would work in human cells if the RNA strands were provided as pre-sized duplexes of about 19 nucleotide pairs, and RNAi worked particularly well with small unpaired 3′ extensions on the end of each strand (Elbashir et al. Nature 411: 494-498 (2001)). In this report, “short interfering RNA” (siRNA, also referred to as small interfering RNA) were applied to cultured cells by transfection in oligofectamine micelles. These RNA duplexes were too short to elicit sequence-nonspecific responses like apoptosis, yet they efficiently initiated RNAi. Many laboratories then tested the use of siRNA to knock out target genes in mammalian cells. The results demonstrated that siRNA works quite well in most instances.

For purposes of reducing the activity of ANCCA, siRNAs to the gene encoding ANCCA can be specifically designed using computer programs. A program, siDESIGN from Dharmacon, Inc. (Lafayette, Colo.), permits predicting siRNAs for any nucleic acid sequence, and is available on the World Wide Web at dharmacon.com. Programs for designing siRNAs are also available from others, including Genscript (available on the Web at genscript.com/ssl-bin/app/rnai) and, to academic and non-profit researchers, from the Whitehead Institute for Biomedical Research found on the worldwide web at “jura.wi.mit.edu/pubint/http://iona.wi.mit.edu/siRNAext/.”

Alternatively, siRNA can be generated using kits which generate siRNA from the gene. For example, the “Dicer siRNA Generation” kit (catalog number T510001, Gene Therapy Systems, Inc., San Diego, Calif.) uses the recombinant human enzyme “dicer” in vitro to cleave long double stranded RNA into 22 bp siRNAs. By having a mixture of siRNAs, the kit permits a high degree of success in generating siRNAs that will reduce expression of the target gene. Similarly, the Silencer™ siRNA Cocktail Kit (RNase III) (catalog no. 1625, Ambion, Inc., Austin, Tex.) generates a mixture of siRNAs from dsRNA using RNase III instead of dicer. Like dicer, RNase III cleaves dsRNA into 12-30 bp dsRNA fragments with 2 to 3 nucleotide 3′ overhangs, and 5′-phosphate and 3′-hydroxyl termini. According to the manufacturer, dsRNA is produced using T7 RNA polymerase, and reaction and purification components included in the kit. The dsRNA is then digested by RNase III to create a population of siRNAs. The kit includes reagents to synthesize long dsRNAs by in vitro transcription and to digest those dsRNAs into siRNA-like molecules using RNase III. The manufacturer indicates that the user need only supply a DNA template with opposing T7 phage polymerase promoters or two separate templates with promoters on opposite ends of the region to be transcribed.

The siRNAs can also be expressed from vectors. Typically, such vectors are administered in conjunction with a second vector encoding the corresponding complementary strand. Once expressed, the two strands anneal to each other and form the functional double stranded siRNA. One exemplary vector suitable for use in the invention is pSuper, available from OligoEngine, Inc. (Seattle, Wash.). In some embodiments, the vector contains two promoters, one positioned downstream of the first and in antiparallel orientation. The first promoter is transcribed in one direction, and the second in the direction antiparallel to the first, resulting in expression of the complementary strands. In yet another set of embodiments, the promoter is followed by a first segment encoding the first strand, and a second segment encoding the second strand. The second strand is complementary to the palindrome of the first strand. Between the first and the second strands is a section of RNA serving as a linker (sometimes called a “spacer”) to permit the second strand to bend around and anneal to the first strand, in a configuration known as a “hairpin.”

The formation of hairpin RNAs, including use of linker sections, is well known in the art. Typically, an siRNA expression cassette is employed, using a Polymerase III promoter such as human U6, mouse U6, or human H1. The coding sequence is typically a 19-nucleotide sense siRNA sequence linked to its reverse complementary antisense siRNA sequence by a short spacer. Nine-nucleotide spacers are typical, although other spacers can be designed. For example, the Ambion website indicates that its scientists have had success with the spacer TTCAAGAGA (SEQ ID NO:51). Further, 5-6 T's are often added to the 3′ end of the oligonucleotide to serve as a termination site for Polymerase III. See also, Yu et al., Mol Ther 7(2):228-36 (2003); Matsukura et al., Nucleic Acids Res 31(15):e77 (2003).

As an example, the siRNA targets identified above can be targeted by hairpin siRNA as follows. To attack the same targets by short hairpin RNAs, produced by a vector (permanent RNAi effect), sense and antisense strand can be put in a row with a loop forming sequence in between and suitable sequences for an adequate expression vector to both ends of the sequence.

In addition to siRNAs, other means are known in the art for inhibiting the expression of antisense molecules, ribozymes, and the like are well known to those of skill in the art. The nucleic acid molecule can be a DNA probe, a riboprobe, a peptide nucleic acid probe, a phosphorothioate probe, or a 2′-O methyl probe.

Generally, to assure specific hybridization, the inhibitory nucleic acid sequence is substantially complementary to the target sequence (e.g., at least about 95%, 96%, 97%, 98% or 99% complementary). In certain embodiments, the inhibitory nucleic acid sequence is exactly complementary to the target sequence. The inhibitory nucleic acid polynucleotides may also include, however, nucleotide substitutions, additions, deletions, transitions, transpositions, or modifications, or other nucleic acid sequences or non-nucleic acid moieties so long as specific binding to the relevant target sequence corresponding to the ANCCA gene is retained as a functional property of the polynucleotide. In one embodiment, the inhibitory nucleic acid molecules form a triple helix-containing, or “triplex” nucleic acid. Triple helix formation results in inhibition of gene expression by, for example, preventing transcription of the target gene (see, e.g., Cheng et al., 1988, J. Biol. Chem. 263:15110; Ferrin and Camerini-Otero, 1991, Science 354:1494; Ramdas et al., 1989, J. Biol. Chem. 264:17395; Strobel et al., 1991, Science 254:1639; and Rigas et al., 1986, Proc. Natl. Acad. Sci. U.S.A. 83:9591)

Antisense molecules can be designed by methods known in the art. For example, Integrated DNA Technologies (Coralville, Iowa) makes available a program found on the worldwide web “biotools.idtdna.com/antisense/AntiSense.aspx”, which will provide appropriate antisense sequences for nucleic acid sequences up to 10,000 nucleotides in length.

In another embodiment, ribozymes can be designed to cleave the mRNA at a desired position. (See, e.g., Cech, 1995, Biotechnology 13:323; and Edgington, 1992, Biotechnology 10:256 and Hu et al., PCT Publication WO 94/03596).

The antisense nucleic acids (DNA, RNA, modified, analogues, and the like) can be made using any suitable method for producing a nucleic acid, such as the chemical synthesis and recombinant methods disclosed herein and known to one of skill in the art. In one embodiment, for example, antisense RNA molecules of the invention may be prepared by de novo chemical synthesis or by cloning. For example, an antisense RNA can be made by inserting (ligating) an ANCCA gene sequence in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention.

It will be appreciated that the oligonucleotides can be made using nonstandard bases (e.g., other than adenine, cytidine, guanine, thymine, and uridine) or nonstandard backbone structures to provides desirable properties (e.g., increased nuclease-resistance, tighter-binding, stability or a desired Tm). Techniques for rendering oligonucleotides nuclease-resistant include those described in PCT Publication WO 94/12633. A wide variety of useful modified oligonucleotides may be produced, including oligonucleotides having a peptide-nucleic acid (PNA) backbone (Nielsen et al., 1991, Science 254:1497) or incorporating 2′-O-methyl ribonucleotides, phosphorothioate nucleotides, methyl phosphonate nucleotides, phosphotriester nucleotides, phosphorothioate nucleotides, phosphoramidates.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996). Another subsequence, the h (hydrophobic) domain of signal peptides, was found to have similar cell membrane translocation characteristics (see, e.g., Lin et al., J. Biol. Chem. 270:14255-14258 (1995)). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

More recently, it has been discovered that siRNAs can be introduced into mammals without eliciting an immune response by encapsulating them in nanoparticles of cyclodextrin. Information on this method can be found on the worldwide web at “nature.com/news/2005/050418/full/050418-6.html.”

In another method, the nucleic acid is introduced directly into superficial layers of the skin or into muscle cells by a jet of compressed gas or the like. Methods for administering naked polynucleotides are well known and are taught, for example, in U.S. Pat. No. 5,830,877 and International Publication Nos. WO 99/52483 and 94/21797. Devices for accelerating particles into body tissues using compressed gases are described in, for example, U.S. Pat. Nos. 6,592,545, 6,475,181, and 6,328,714. The nucleic acid may be lyophilized and may be complexed, for example, with polysaccharides to form a particle of appropriate size and mass for acceleration into tissue. Conveniently, the nucleic acid can be placed on a gold bead or other particle which provides suitable mass or other characteristics. Use of gold beads to carry nucleic acids into body tissues is taught in, for example, U.S. Pat. Nos. 4,945,050 and 6,194,389.

The nucleic acid can also be introduced into the body in a virus modified to serve as a vehicle without causing pathogenicity. The virus can be, for example, adenovirus, fowlpox virus or vaccinia virus.

miRNAs and siRNAs differ in several ways: miRNA derive from points in the genome different from previously recognized genes, while siRNAs derive from mRNA, viruses or transposons, miRNA derives from hairpin structures, while siRNA derives from longer duplexed RNA, miRNA is conserved among related organisms, while siRNA usually is not, and miRNA silences loci other than that from which it derives, while siRNA silences the loci from which it arises. Interestingly, miRNAs tend not to exhibit perfect complementarity to the mRNA whose expression they inhibit. See, McManus et al., supra. See also, Cheng et al., Nucleic Acids Res. 33(4):1290-7 (2005); Robins and Padgett, Proc Natl Acad Sci USA. 102(11):4006-9 (2005); Brennecke et al., PLoS Biol. 3(3):e85 (2005). Methods of designing miRNAs are known. See, e.g., Zeng et al., Methods Enzymol. 392:371-80 (2005); Krol et al., J Biol. Chem. 279(40):42230-9 (2004); Ying and Lin, Biochem Biophys Res Commun. 326(3):515-20 (2005).

The inhibitory nucleic acid can be targeted to the tissue to be treated. Targeting can be accomplished using any method known in the art. Targeting of inhibitor nucleic acids is discussed, e.g., in Jackson and Linsley, Nat Rev Drug Discov. (2010) 9(1):57-67; Pappas, et al., Expert Opin Ther Targets. 2008 January; 12(1):115-27; Li, et al., AAPS J. (2009) 11(4):747-57.

9. Administering Therapy to the Patient

Upon a positive diagnosis of breast cancer, progression of breast cancer and/or cancer metastasis or confirmation of a diagnosis of breast cancer, progression of breast cancer and/or cancer metastasis, the present methods may further include the step of determining an appropriate therapeutic or preventative regimen for the patient, and/or administering an appropriate therapy based on the diagnosis of breast cancer, breast cancer progression and/or cancer metastasis associated with, correlated with, and/or caused by overexpression of ANCCA.

In various embodiments, the appropriate therapeutic or preventative regimen can be based on present clinical treatments available to patients diagnosed with a breast cancer, and can include known treatment regimens, including chemotherapy, radiation, surgery, biotherapeutics (e.g., immunotoxin therapy, antibody therapy, cytokines, etc.), or other known treatments as appropriate and presently prescribed for treatment of breast cancer.

In some embodiments, the established breast cancer therapy regimen is co-administered with an agent that inhibits ANCCA protein activity.

In some embodiments, the established breast cancer therapy regimen is co-administered with an inhibitory nucleic acid that inhibits expression of ANCCA, as described above.

In therapeutic applications, the ANCCA inhibitors can be administered to an individual already suffering from or at risk for developing breast cancer and/or breast cancer metastasis. Compositions that contain ANCCA inhibitors are administered to a patient in an amount sufficient to prevent, reduce, inhibit and/or delay the proliferation and/or growth of a breast cancer and to eliminate or at least partially arrest symptoms and/or complications. An amount adequate to accomplish this is defined as a “therapeutically effective dose.” Amounts effective for this use will depend on, e.g., the inhibitor composition, the manner of administration, the stage and severity of the disease being treated, the weight and general state of health of the patient, and the judgment of the prescribing physician Inhibitors of ANCCA activity can be administered chronically or acutely to prevent, reduce, inhibit and/or delay the proliferation and/or growth of a breast cancer, breast cancer progression and/or breast cancer metastasis. In certain instances, it will be appropriate to administer an inhibitor of ANCCA activity prophylactically, for instance in subjects in remission or suspected of having breast cancer metastasis.

Alternatively, DNA or RNA that inhibits expression of one or more sequences encoding ANCCA, such as an antisense nucleic acid, a small-interfering nucleic acid (i.e., siRNA), a micro RNA (miRNA), or a nucleic acid that encodes a peptide that blocks expression or activity of ANCCA can be introduced into patients to achieve inhibition. U.S. Pat. No. 5,580,859 describes the use of injection of naked nucleic acids into cells to obtain expression of the genes which the nucleic acids encode.

Therapeutically effective amounts of an ANCCA inhibitor composition generally range for the initial administration (that is for therapeutic or prophylactic administration) from about 0.1 μg to about 10 mg of ANCCA inhibitor for a 70 kg patient, usually from about 1.0 μg to about 1 mg, for example, between about 10 μg to about 0.1 mg (100 μg). Typically, lower doses are initially administered and incrementally increased until a desired efficacious dose is reached. These doses can be followed by repeated administrations over weeks to months depending upon the patient's response and condition by evaluating symptoms associated with breast cancer.

For prophylactic use, administration should be given to subjects at risk for or suspected of having breast cancer, breast cancer progression and/or recurrence and/or breast cancer metastasis. Therapeutic administration may begin at the first sign of disease or the detection or shortly after diagnosis of recurrence and/or metastasis. This is often followed by repeated administration until at least symptoms are substantially abated and for a period thereafter.

The ANCCA inhibitors for therapeutic or prophylactic treatment are intended for parenteral, topical, oral or local administration. Preferably, the compositions are formulated for oral administration. In certain embodiments, the pharmaceutical compositions are administered parenterally, e.g., intravenously, intranasally, inhalationally, subcutaneously, intradermally, or intramuscularly. Compositions of the invention are also suitable for oral administration. Thus, the invention provides compositions for parenteral administration which comprise a solution of the ANCCA inhibiting agent dissolved or suspended in an acceptable carrier, preferably an aqueous carrier. A variety of aqueous carriers may be used, e.g., water, buffered water, 0.9% saline, 0.3% glycine or another suitable amino acid, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of ANCCA inhibiting agents of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The ANCCA inhibitors may also be administered via liposomes, which can be designed to target the conjugates to a particular tissue, for example, breast tissue and tissues infiltrated by metastatic tumors, as well as increase the half-life of the peptide composition. Liposomes include emulsions, foams, micelles, insoluble monolayers, liquid crystals, phospholipid dispersions, lamellar layers and the like. In these preparations, the peptide, nucleic acid or organic compound to be delivered is incorporated as part of a liposome, alone or in conjunction with a molecule which binds to, e.g., a receptor prevalent among the desired cells, or with other therapeutic compositions. Thus, liposomes filled with a desired peptide, nucleic acid, small molecule or conjugate of the invention can be directed to the site of, for example, immune cells, leukocytes, lymphocytes, myeloid cells or endothelial cells, where the liposomes then deliver the selected ANCCA inhibitor compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid liability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028.

The targeting of liposomes using a variety of targeting agents is well known in the art (see, e.g., U.S. Pat. Nos. 4,957,773 and 4,603,044). For targeting to desired cells, a ligand to be incorporated into the liposome can include, e.g., antibodies or fragments thereof specific for cell surface determinants of the target cells. A liposome suspension containing an ANCCA inhibitor may be administered intravenously, locally, topically, etc. in a dose which varies according to, inter alia, the manner of administration, the conjugate being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more conjugates of the invention, and more preferably at a concentration of 25%-75%.

For aerosol administration, the inhibitors are preferably supplied in a suitable form along with a surfactant and propellant. Typical percentages of ANCCA inhibitors are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

An effective anti-cancer treatment is indicated by a decrease in observed breast cancer symptoms, and/or tumor burden, as measured according to a clinician or reported by the patient. Alternatively, methods for detecting levels of specific ANCCA activities can be used. Standard assays for detecting ANCCA activity, e.g., ATPase activity, binding to substrate, binding to nuclear receptor, multimerization, are known in the art. Again, an effective anti-cancer treatment is indicated by a substantial reduction in activity of ANCCA. As used herein, a “substantial reduction” in ANCCA activity refers to a reduction of at least about 30% in the test sample compared to an untreated control. Preferably, the reduction is at least about 50%, more preferably at least about 75%, and most preferably ANCCA activity levels are reduced by at least about 90% in a sample from a treated mammal compared to an untreated control. In some embodiments, the ANCCA activity is completely inhibited.

10. Kits

The present invention also includes an ANCCA-detection reagent, e.g., a nucleic acid that specifically binds to or identifies one or more ANCCA nucleic acids, including oligonucleotide sequences which are complementary to a portion of an ANCCA nucleic acid, or an antibody that binds to one or more proteins encoded by an ANCCA nucleic acid. The detection reagents can be packaged together in the form of a kit. For example, the detection reagents can be packaged in separate containers, e.g., a nucleic acid or antibody (either bound to a solid matrix or packaged separately with reagents for binding them to the matrix), a control reagent (positive and/or negative), and/or a detectable label. Instructions (e.g., written, tape, VCR, CD ROM, etc.) for carrying out the assay and correlating increased expression of ANCCA with an affirmative diagnosis of cancer can also be included in the kit. The assay format of the kit can be a Northern hybridization or a sandwich ELISA, both of which are known in the art. See, for example, Sambrook and Russell, Molecular Cloning: A Laboratory Manual, 3.sup.rd Edition, 2001, Cold Spring Harbor Laboratory Press; and Harlow and Lane, Using Antibodies, supra.

For example, an ANCCA detection reagent can be immobilized on a solid matrix, for example, a porous strip, to form at least one ANCCA detection site. The measurement or detection region of the porous strip can include a plurality of sites, each containing a nucleic acid. A test strip can also contain sites for negative and/or positive controls. Alternatively, control sites can be located on a separate strip from the test strip. Optionally, the different detection sites can contain different amounts of immobilized nucleic acids, i.e., a higher amount in the first detection site and lesser amounts in subsequent sites. Upon the addition of test sample, the number of sites displaying a detectable signal provides a quantitative indication of the amount of ANCCA present in the sample. The detection sites can be configured in any suitably detectable shape and are typically in the shape of a bar or dot spanning the width of a test strip.

In various embodiments, the kits comprise instructions correlating the overexpression of ANCCA in a biological sample (e.g., breast tissue, a metastatic tumor) with breast cancer, progression of breast cancer and/or breast cancer metastasis.

In other embodiments, the invention provides kits containing an inhibitory nucleic acid that specifically hybridizes to and inhibits expression of an ANCCA nucleic acid and instructions for administration to a patient to reduce, inhibit or prevent growth of a breast cancer or tumor that is associated with or caused by overexpression of ANCCA.

### EXAMPLES

The following examples are offered to illustrate, but not to limit the claimed invention.

### Example 1

### Materials and Methods

Cell Culture, siRNA Transfection, RNA Analysis and Western Blotting.

Human breast cancer cell lines were obtained from ATCC. HMECs were from Lonza/Clonetics. RNA analysis and Western blotting were performed as previously described (10). For transfection of siRNAs, cells plated at 2.0×10⁵ cells per well in six-well plates were transfected using Dharmafect 1 (Dharmacon) according to the manufacturer's instructions. siRNA targeting ANCCA or control siRNA were used at 100 nM concentration. Details of cell lines and their culture conditions, antibodies used for Western blotting, and primers for RNA analysis are described herein.

Cell Proliferation and Soft-Agar Colony Formation Assays.

HCC 1937 cells were seeded at density of 1.5×10⁵ cells per well of a six-well plate and maintained in full growth medium for 24 hrs before being infected with equal titers of adeno-ANCCA-HA or adeno-vector adenoviruses (8). MDA-MB 468 cells were plated at a density of 2.0×10⁵ cells per well in six well plates, and were transfected with siRNA 24 hours later as described above. Medium was changed every other day after transfection and cell proliferation was measured by cell counting of coded samples in triplicates. For colony formation in soft agar, five thousand cells were seeded in each well of six-well plates in medium containing 0.4% SeaPlaque® Agarose (Lonza) on top of base medium containing 0.8% agarose. Colonies were stained 4 to 5 weeks later with MTT (Sigma Aldrich) and counted using a light microscope.

TUNEL Assay.

Cells were plated and transfected with siRNA 24 hours later as described above. Cells were plated 72 hrs later on glass chamber slides (Nunc) and processed for TUNEL assay using the In Situ Cell Death Detection kit (Roche Diagnostics) according to manufacturer's protocol. Slides were mounted with VECTASHIELD Mounting Media (Vector Laboratories). Images were acquired using Olympus BX6. For quantification of TUNEL-positive cells, 10 random fields of the same condition were counted and averaged.

Tumor Specimens and Immunohistochemistry.

Archival paraffin-embedded primary tumors samples were from 225 women diagnosed during 1998-2004 with breast carcinoma and treated at UC Davis Medical Center. Other information on the patients and tumors are described in Table 1.

TABLE 1
Clinico-pathological Characteristics of Breast Carcinoma Patients

| | IDC/DCTS, # (%) |
|---|---|
| Age (yrs) | |
| mean | 57.3 ± 0.9* |
| range | 26.94 |
| Tumor size (cm) | |
| mean | 1.7 ± 0.14* |
| range | 0.1-9.0 |
| Lymph Node | |
| negative | 74 (62%) |
| positive | 45 (38%) |
| ER | |
| negative | 142 (59%) |
| positive | 100 (41%) |
| PR | |
| negative | 123 (51%) |
| positive | 119 (49%) |
| HER2 | |
| negative | 133 (73%) |
| positive | 50 (27%) |
| Ki67 | |
| low (<10%) | 47 (22%) |
| high (>10%) | 168 (78%) |
| P53 | |
| negative (<10%) | 87 (68%) |
| positive (>10%) | 41 (32%) |

*Patient's age and tumor size are shown as mean ± standard error of mean

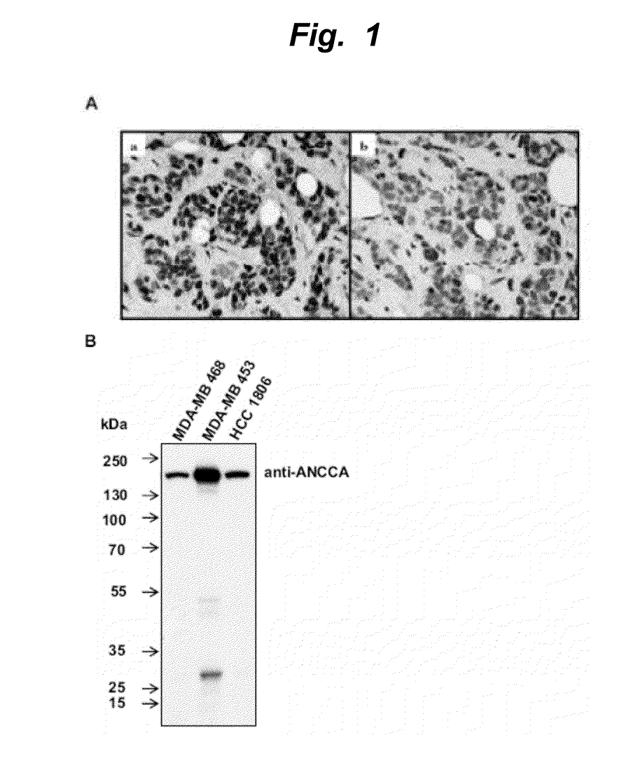

Five μm sections of the tumor blocks were first subjected to de-paraffinization and then antigen retrieval in 0.01 mM sodium citrate buffer (pH 6.0) in a microwave oven at 1000 W for 5 minutes and then at 100 w for 30 min. Breast tumor tissue microarray or TMAs (BR1002, BR961, and BR208) were obtained from US BioMax, Inc, baked at 600 C for 2 hrs and processed for immunostaining TMA for EZH2 and ANCCA correlation study has been described (7). Nonspecific immunoglobulin binding was blocked using 10% fetal bovine serum in PBS for 30 min at room temperature. Slides were then incubated at room temperature for 30 min with anti-ANCCA antibody (at 1:300), anti-Ki-67 (NeoMarker, at 1:300), and anti-EZH2 (AC22, Cell Signaling, at 1:50). Anti-ANCCA antibody was raised in rabbit (Covance) and affinity-purified by using GST-ANCCA N-terminus (aa 2-264) expressed and purified from E. coli. Its specificity for IHC was determined using a panel of cell lines and xenograft tumors (9) and is also shown in FIG. 1. After incubation with primary antibody, the sections were washed and incubated with biotin-conjugated secondary antibodies for 30 min followed by incubation with avidin DHbiotinylated horseradish peroxidase and developed using diaminobenzidine (DAB) substrate kit (Vector Laboratories) and counterstained using Gill's Hematoxylin. Images were acquired using an Olympus microscope with DPController software. The percentage of positively stained nuclei was scored as follows: 0-10%, score 0; 11-25%, score 1; 26-50%, score 2; >50%, score 3. The immunoreactivity was evaluated by at least two different investigators with no prior knowledge of patient data.

Statistical Analyses and Analysis of Microarray Gene Expression Data Sets.

Values of patient age and tumor size are presented as mean±SE. Association between ANCCA immunoreactivity and other clinico-pathological parameters was evaluated using Pearson χ2 test. Survival curves were generated using the Kaplan-Meier method taking into account censored data. The curves were compared using the log-rank test (Mantel-Cox). For other assays and analysis of gene transcripts in tumor data sets using Oncomine (on the internet at oncomine.org), Student's t-test was used for comparison of experimental groups. Statistical analysis was performed using the SPSS software (version 18, SPSS Inc.). P values of less than 0.05 were considered significant. The method for analysis of microarray gene expression data sets is provided in herein.

Cell Culture.

MDA-MB 231, MDA-MB 436, MDA-MB 468, MDA-MB 453 and HCC1937 were obtained from ATCC and cultured in DMEM supplemented with 10% FBS (Omega) for MDA-MB 231, or RPMI supplemented with 10% FBS for the other cell lines. Normal human mammary epithelial cells (HMECs) were obtained from Lonza/Clonetics and cultured in MEGM with BulletKit supplied from the manufacture. HMECs were harvested for Western analysis after being passed two times since the initial thaw and plating.

Antibodies for Western Blotting and ChIP.

Antibodies against Cdc6 (D-1), Cyclin D1 (H-295), Cyclin E1 (E-4), E2F1 (C-20 or KH-95), MCMI (G-7), PCNA (sc-56), VEGF (sc-152) and SUZ12 (P-15) were from Santa Cruz Biotechnology. Antibodies against Cyclin A2 (611269) and Cyclin Bl (610219) were from Beckton Dickinson. Antibodies against EZH2 (AC22), AKT1 (9272), pAKT1 (40513), IRS2 (4502), SGK (3272) were from Cell Signaling Technology. Beta-Actin (AC-74) was from Sigma. Anti-ANCCA antibody was raised in rabbit (Covance) and affinity-purified by using GSTANCCA N-terminus (aa 2-264) expressed and purified from E. coli.

Analysis of ANCCA/ATAD2 Expression Association with Other Genes in Microarray Gene Expression Data Sets.

In order to investigate ANCCA/ATAD2 transcript expression patterns in clinical breast cancer tissues, we utilized the dataset obtained in a study which profiled 198 patient samples from the TRANSBIG Consortium with Affymetrix HG-U133A GeneChip arrays (Desmedt, et al., Clin Cancer Res (2007) 13: 3207-14; Buyse, et al., J Natl Cancer Inst (2006) 98: 1183-92). The raw data (CEL files) and the sample and data relationship information were downloaded from ArrayExpress (European Bioinformatics Institute, Cambridge, UK) (Brazma, et al., Nucleic Acids Res (2003) 31: 68-71). Data analysis was performed using GeneSpring GX (version 10) software (Agilent Technologies, Santa Clara, Calif.). Briefly, probe-set expression intensities were obtained using robust multi-array average (RMA) (Irizarry, et al, Biostatistics (2003) 4: 249-64) for probe summarization of quantile-normalized, background-adjusted, and log-transformed perfect match (PM) probe intensity values. The data was then filtered to retain only those probesets having expression values that exceeded the 20% lower cut-off threshold in at least one of the samples. Subsequently, transcripts co-expressed with ANCCA/ATAD2 were identified using the Spearmann rank correlation test. Hierarchical clustering was then performed to further organize the ATAD2 co-expression cluster based on similarities of expression between genes and conditions (i.e., ER status).

Primers used for RT-PCR analysis:

| (SEQ ID NO: 1) | |
|---|---|
| RT-NCAPG-r1 | TCCTATCTTTTACTTGCTCC |
| (SEQ ID NO: 2) | |
| RT-NCAPG-f1 | CAAGACTTCCCAAGATTATC |
| (SEQ ID NO: 3) | |
| RT-TOP2A-r1 | GAGTCAAAGGTTGGGTTTTC |
| (SEQ ID NO: 4) | |
| RT-TOP2A-f1 | GTGGGAAGTGTGTTTAACTAT |
| (SEQ ID NO: 5) | |
| RT-MCM10-r1 | GTTGGGGTTATTTAAGGCTG |
| (SEQ ID NO: 6) | |
| RT-MCM10-f1 | TGAATACCACTGGCATGATG |
| (SEQ ID NO: 7) | |
| RT-SMC2-r2 | TTCTTCCTTACCTGAATGAC |
| (SEQ ID NO: 8) | |
| RT-SMC2-f2 | TTATGGTCCAATTATTGTGG |
| (SEQ ID NO: 9) | |
| RT-DLGAP5-r2 | TTTGTTGCTTGAGACTCATC |
| (SEQ ID NO: 10) | |
| RT-DLGAP5-f2 | AAATGGAAAACTTACCTGAG |
| (SEQ ID NO: 11) | |
| RT-KIF15-r1 | TTTTTGCCTTTCAGATCCTG |
| (SEQ ID NO: 12) | |
| RT-KIF15-f1 | GAAAGAGTTTCCTTTGTAAGT |
| (SEQ ID NO: 13) | |
| RT-KIF23 r1 | ACTCTGCACCATCTGGTTGG |
| (SEQ ID NO: 14) | |
| RT-KIF23-f1 | CATGCCATCACAGTATCTGTT |
| (SEQ ID NO: 15) | |
| RT-SMC4-r2 | GCAAACATTACTCAAGGTAT |
| (SEQ ID NO: 16) | |
| RT-SMC4-f2 | CTTTCTCTGAAGGAATCATG |
| (SEQ ID NO: 17) | |
| RT-DLGAP5-f1 | AAGGGTATTTCTTGTAAAGTC |
| (SEQ ID NO: 18) | |
| RT-DLGAP5-r1 | AAAAGCATTGGCACTTCTGG |
| (SEQ ID NO: 19) | |
| RT-KIF4A-f1 | AATGAGCATGAGGATGGTGAT |
| (SEQ ID NO: 20) | |
| RT-KIF4A-r1 | TCCGTTCAACAGTGCCCAAG |
| (SEQ ID NO: 21) | |
| RT-BUB1-r2 | GCAATAGCATCTGGTGGATC |
| (SEQ ID NO: 22) | |
| RT-BUB1-f2 | CAAGGGATGGAAAATTCAGTC |
| (SEQ ID NO: 23) | |
| RT-TAF2-r1 | AAAGTGTGAAGTACAAGTCC |
| (SEQ ID NO: 24) | |
| RT-TAF2-f1 | ACTAAAGTGGACAGAAGTTAT |
| (SEQ ID NO: 25) | |
| RT-KIF11-r1 | ATTATACCAGCCAAGGGATC |
| (SEQ ID NO: 26) | |
| RT-KIF11-f1 | TCCTGTACGAAAAGAAGTTAG |
| (SEQ ID NO: 27) | |
| RT-DSCC1-f1 | GAGTCCTGAAGAAAAGACCG |
| (SEQ ID NO: 28) | |
| RT-DSCC1-f1 | AAGAAGTGTGGCAGCAGAGTG |
| (SEQ ID NO: 29) | |
| RT-RAD51AP1-f1 | AGGACATGGTAATGGATTCC |
| (SEQ ID NO: 30) | |
| RT-RAD51AP1-f1 | TAGCAGAAGTAGCAGCAGCC |
| (SEQ ID NO: 31) | |
| RT-GPSM2-r1 | AAGGAGGTAATGTGTGTTTC |
| (SEQ ID NO: 32) | |
| RT-GPSM2-f1 | ACAATCTTGGGAATGTGTATC |
| (SEQ ID NO: 33) | |
| RT-CEP55-f1 | AGCCATTAGTCACTTTCCAAG |
| (SEQ ID NO: 34) | |
| RT-CEP55-r1 | CACAGCAGCCTAAAAGATAC |
| (SEQ ID NO: 35) | |
| RT-ECT2-f1 | CTTTACAAAGCCCACTTTAA |
| (SEQ ID NO: 36) | |
| RT-ECT2-f1 | AGAGTGCTGATTTAGAAGAA |
| (SEQ ID NO: 37) | |
| RT-cycA2-f1 | CCCCCAGAAGTAGCAGAGTTTGTG |
| (SEQ ID NO: 38) | |
| RT-cycA2-r1 | GCTTTGTCCCGTGACTGTGTAGAG |
| (SEQ ID NO: 39) | |
| RT-actin-f1 | GAGAAAATCTGGCACCACACC |
| (SEQ ID NO: 40) | |
| RT-actin-r1 | ATACCCCTCGTAGATGGGCAC |
| (SEQ ID NO: 41) | |
| RT2-GAPDH | GAAATCCCATCACCATCTTCCAG |
| (SEQ ID NO: 42) | |
| RT1-GAPDH | ATGAGTCCTTCCACGATACCAAAG |
| (SEQ ID NO: 43) | |
| cycE2-RT-s | ACTGACTGCTGCTGCCTTGTGC |
| (SEQ ID NO: 44) | |
| cycE2-RT-as | TCGGTGGTGTCATAATGCCTCC |
| (SEQ ID NO: 45) | |
| RT-ANCCA-f1 | CACCGAGTACTCCTGTGGCTTG |
| (SEQ ID NO: 46) | |
| RT-ANCCA-r1 | TCTAGCTCGAGTCATTCGCAGAACAC |
| (SEQ ID NO: 47) | |
| RT-CDC2/A | GCACTTGGCTTCAAAGCTG |
| (SEQ ID NO: 48) | |
| RT-CDC2/C | CCAAGTATTTCTTCAGATCCATGG |

### Results

ANCCA, a “Signature” Gene, is Overexpressed in Greater than 70% of Human Breast Carcinomas and its Overexpression is Associated with Triple-Negative Status.

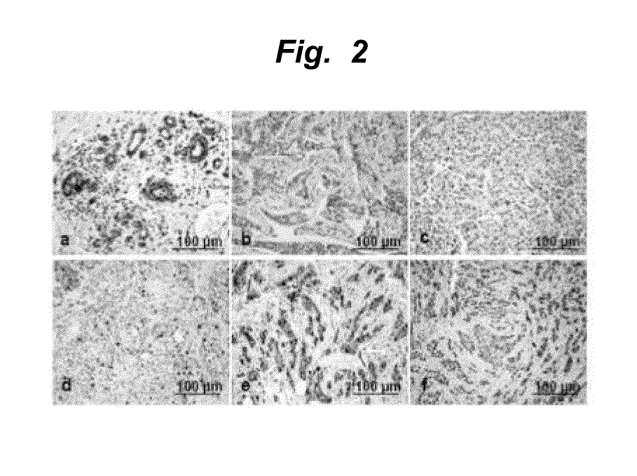

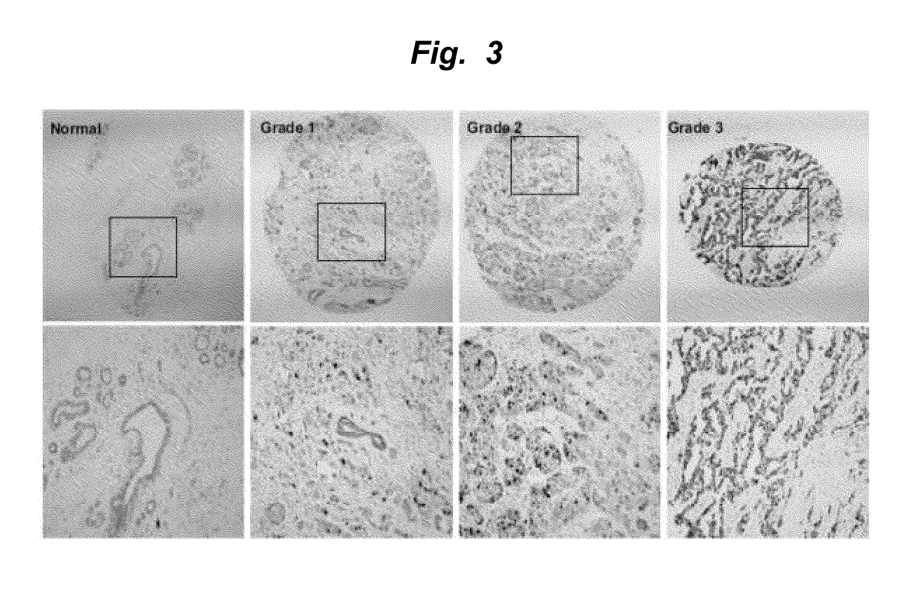

To investigate ANCCA expression in breast cancer, we first performed immunohistochemical (IHC) analysis of ANCCA protein expression in a cohort of 225 primary human breast ductal carcinomas and three independent sets of TMAs containing a total of 131 tumor samples and 24 normal breast tissues. Immuno-reactivity for ANCCA was readily detected in the nuclei of a large subset of tumor tissues while little or no staining was observed in the normal breast tissues or tumor-adjacent stroma (FIG. 2 and FIG. 3). When compared to normal breast tissue, over 70% of all tumor samples examined showed increased levels of ANCCA protein expression (Table 2 and Table 3).

TABLE 2
Tumor characteristics of 200 cases of primary breast carcinoma analyzed
by IHC

| | ANCCA score | | |
|---|---|---|---|
| | 0-1 | 2-3 | |
| | N (%) | N (%) | P* |
| BC all grade | 49 (22) | 176 (78) | <0.0001 |
| Grade | | | |
| I | 13 (45) | 16 (55) | |
| II | 20 (27) | 54 (73) | |
| III | 12 (16) | 63 (84) | <0.0001 |
| Lymph node status | | | |
| Positive | 12 (26) | 34 (74) | |
| Negative | 14 (20) | 57 (80) | 0.4 |
| Hormone Receptor status | | | |
| ER-positive | 31 (37) | 53 (63) | |
| ER-negative | 16 (14) | 10 (86) | 0.0004 |
| PR-positive | 25 (35) | 46 (65) | |
| PR-negative | 17 (13) | 112 (87) | 0.0005 |
| HER2 status | | | |
| Positive | 8 (20) | 31 (80) | |
| Negative | 39 (26) | 112 (74) | 0.63 |
| TN | 8 (11.5) | 62 (88.5) | |
| NTN (all grades) | 41 (31) | 91 (69) | |
| NTN (high grade) | 27 (31.4) | 59 (68.6) | 0.0071 |
| Ki67 (TN) | | | |
| High | 5 (7.5) | 47 (70) | |
| Low | 3 (4.5) | 12 (18) | <0.0001 |

IHC results are presented as frequency with the percentage of cases in parenthesis.
*Pearson's χ² test, for association with grades and triple negative breast cancer was used.
TN, triple negative tumors;
NTN, non-triple negative tumors.

TABLE 3
Tumor characteristics of 131 nonoverlapping cases of breast carcinoma
and 24 normal breast tissue samples from three TMAs

| | | | Breast | | |
|---|---|---|---|---|---|
| | Normal, #(%) | | Carcinoma, #(%) | | |
| ANCCA score | Negative | Positive | Negative | Positive | P value* |
| BR1002 | 9 (90) | 1 (10) | 9 (26) | 26 (74) | |
| BR961 | 5 (100) | 0 (0) | 6 (17) | 30 (83) | |
| BR208 | 7 (88) | 1 (12) | 11 (18) | 49 (82) | <0.0001 |

Samples were considered negative for ANCCA expression if less then 10% of nuclei showed positive staining.
*Pearson's χ² test was used for statistical analysis

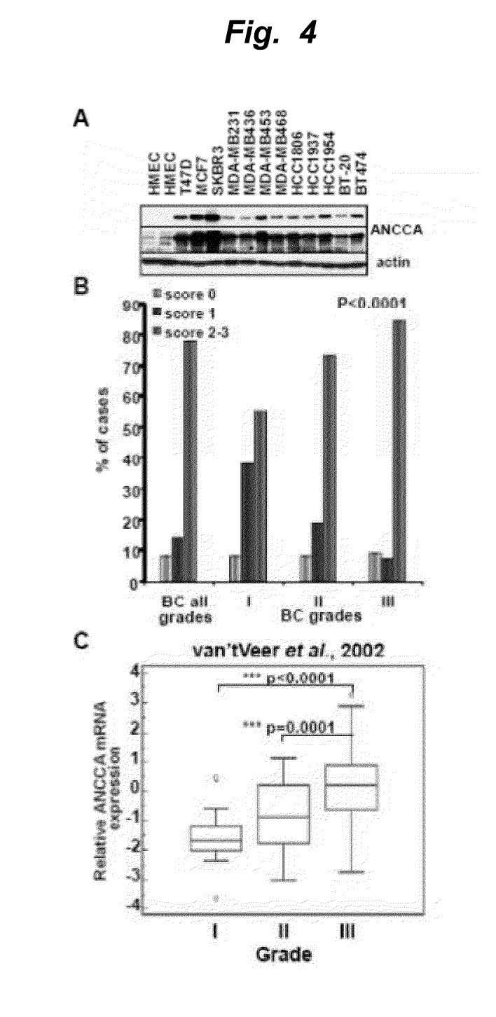

Interestingly, although a large proportion (63%) of ERa-positive tumors displayed ANCCA overexpression, a much stronger association for its overexpression in ERα- and PR-negative tumors was observed (Table 1). More importantly, high levels of ANCCA associate significantly (P=0.0071) with ERα, PR- and HER2-negative status, as over 88% of all triple-negative samples showed high expression of ANCCA protein. Moreover, compared to normal human breast epithelial cells (HMECs), elevated levels of ANCCA proteins (˜170 kD) was observed in all of the breast cancer cell lines examined (FIG. 4A and FIG. 1). We also interrogated multiple microarray gene expression data sets for ANCCA mRNA change in normal and tumor tissues. Interestingly, ANCCA, listed as pro2000, atad2 or other names, is one of the few genes that overlap between gene signatures identified by several gene profiling studies for tumor classification and prediction of disease outcome (e.g. DCIS to IDC, time to distant metastasis; Table 4).

TABLE 4
ANCCA/PRO2000/Atad2 as a common signature
gene in previous gene expression profiling studies

| Name | Signature | Prediction | |
|---|---|---|---|
| listed | gene # | by signature | Study |
| PRO2000 | 70/231 | short interval to | van't Veer LJ Nature2002 |
| | | distant metastasis | |
| PRO2000 | 85 | transition from | Ma X-j et al PNAS2003 |
| | | DCIS to IDC | |
| PRO2000 | 76 | distant metastasis | Wang Y et al Lancet2005 |
| | | of LN negative | Desmedt C et al |
| | | | CliCanRes2007 |
| | | | Fockens JA et al |
| | | | JCliOne2006 |
| Atad2 | 52 | classify ER-positive | Teschendorff AE et al |
| | | tumors | GenBio2006 |
| Atad2 | 264 | reclassify histologic | Ivshina AV et al |
| | | grades | CanRes2006 |

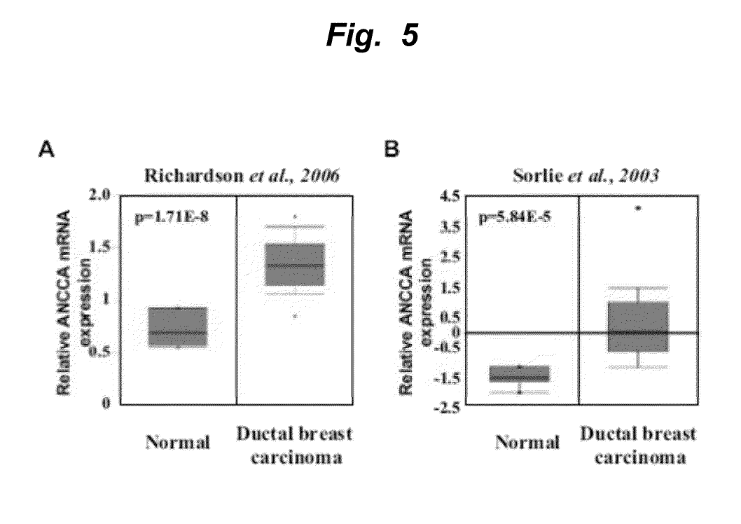

Consistent with our IHC analysis, ANCCA transcript examined in multiple studies displayed much higher levels in tumors than normal breast tissues (FIG. 5), and its high transcript levels strongly associated with ER-negative or triple negative status (P<0.009 and P<0.0001 respectively, Table 5).

TABLE 5
ANCCA mRNA is elevated in hormone-receptor negative and triple
negative tumors*
ANCCA mRNA expression

| | ER status | | |
|---|---|---|---|
| Study | Positive | Negative | P value |
| Wang et al., 2005 (286)** | 0.016 ± 0.022 | 0.209 ± 0.044 | 0.0003 |
| Ivshina et al., 2006 (289) | −0.116 ± 0.026 | 0.116 ± 0.038 | 0.009 |
| Bittner et al., 2005 (336) | 0.8035 ± 0.051 | 1.06 ± 0.067 | 0.0027 |
| | PR status | | |
| | Positive | Negative | |
| Ivshina et al., 2006 (289) | −0.116 ± 0.029 | 0.096 ± 0.051 | 0.0003 |
| Bittner et al., 2005 (336) | 0.8263 ± 0.059 | 0.9838 ± 0.06 | 0.0642 |
| | TN | NTN | |
| Bittner et al., 2005 (336) | 0.639 ± 0.047 | 0.38 ± 0.04 | 0.0001 |
| Bonnefol et al., 2007 (160) | 0.116 ± 0.038 | −0.252 ± 0.056 | <0.0001 |

*Original data were obtained from Bittner et al., 2005; Bonnefol et al., 2007, Ivshina et al., 2006 and Wang et al., 2005 and analyzed using [symbol] .
ANCCA mRNA levels are presented as mean ± standard error of mean.
For statistical analysis, independent samples t-test was used.
TN, triple negative;
NTN, non-triple negative.
**Number of samples in each study is given in brackets. [symbol]
indicates data missing or illegible when filed

ANCCA Overexpression Correlates with Tumor Cell Proliferation and Disease Progression.

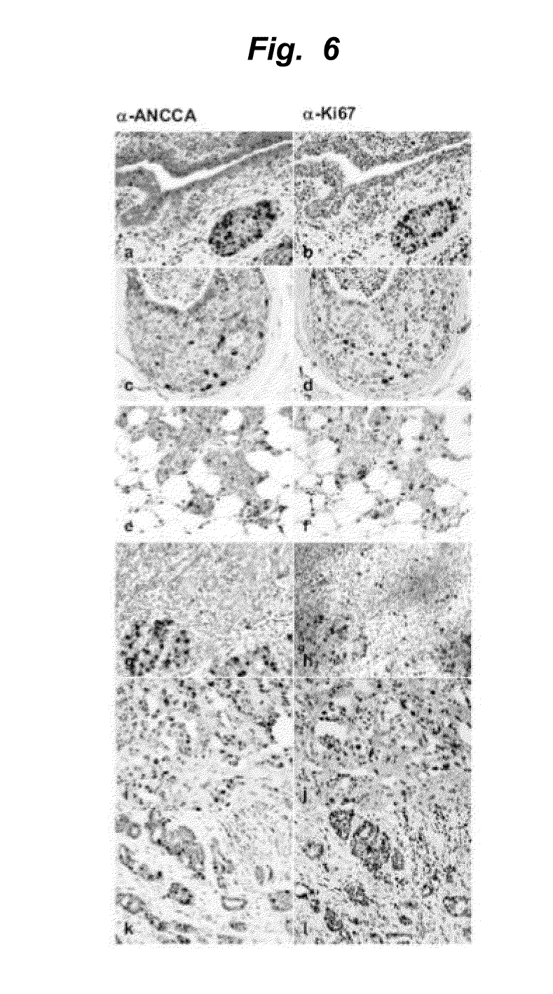

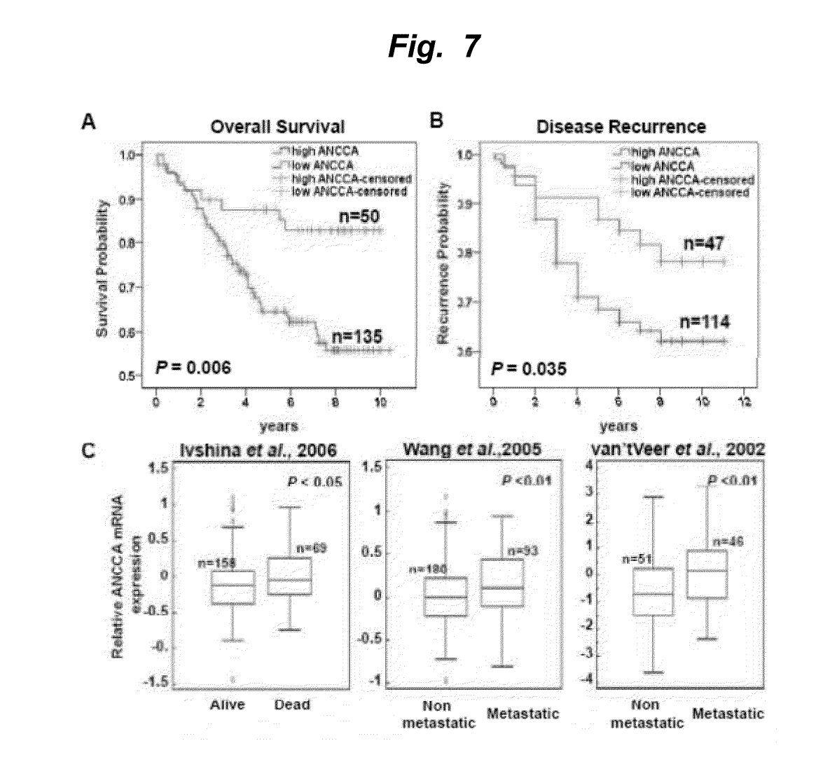

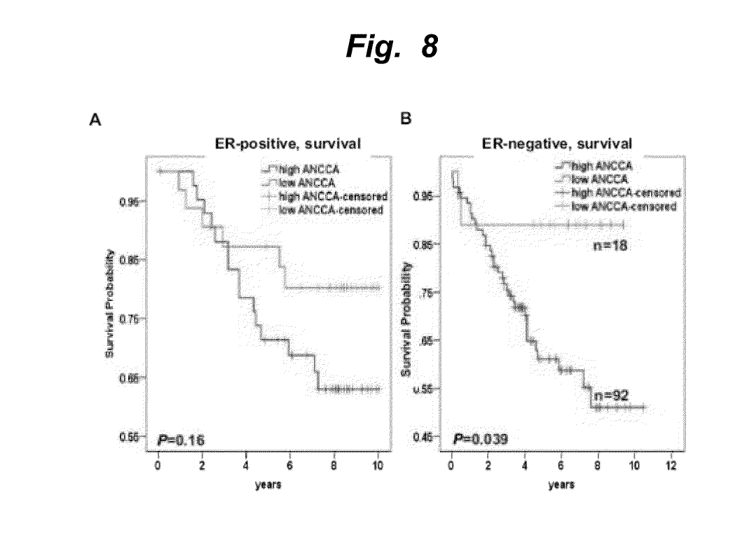

We next assessed the association of ANCCA expression with other clinical and pathological variables (Table 1). Although no clear association was found between ANCCA level and lymph node status, we observed that high levels of ANCCA expression correlate strongly with higher histologic grades (P<0.0001). Overall, more differentiated, low-grade tumors showed little to moderate expression of ANCCA, while less differentiated, high-grade tumors had markedly elevated levels of ANCCA protein (FIGS. 4B and 4C and FIG. 3). Similar results were obtained from analysis of ANCCA transcripts in a cohort of tumors used in a previous study by van't Veer et al. (11) (FIG. 4C). Consistent with the notion that basal-like/triple-negative tumors tend to display high proliferation index (1), the majority (70%) of ANCCA-overexpressing triple negative tumors also showed high Ki-67 staining (P<0.0001, Table 1). IHC analysis of ANCCA and Ki-67 expression in adjacent sections of tumors specimens also revealed a high accordance in their expression in individual tumors (FIG. 6). Highly elevated ANCCA predicts poor overall survival and disease recurrence. We also examined whether strong ANCCA expression is predictive of disease progression by analysis of a cohort of 185 patients with up to 11 years of follow-up information. Tumors were divided into two groups (low ANCCA and high ANCCA) according to ANCCA IHC scores. As shown in FIG. 7A, patients whose tumors showed high ANCCA expression revealed a significantly shorter overall survival period when compared to those with no or weak ANCCA expression (P=0.006 by Cox-Mantel log rank test; 80% survival for high ANCCA group, 2.5 years, and for low ANCCA, 5 years). When the relationship between ANCCA levels and survival was assessed with ER status taken into consideration, their association with poor survival can still be observed in patients with ER-negative tumors (P=0.035, FIG. 8). Likewise, patients with high levels of ANCCA protein had an earlier time to disease recurrence compared to patients with low ANCCA expression (FIG. 7B). Moreover, examination of ANCCA transcripts in the datasets of several “gene signature” studies revealed that high ANCCA mRNA levels associate with high probability of death and tumor metastasis (FIG. 7C).

ANCCA Overexpression Promotes Anchorage-Dependent and -Independent Proliferation and Survival of TNBC Cells.

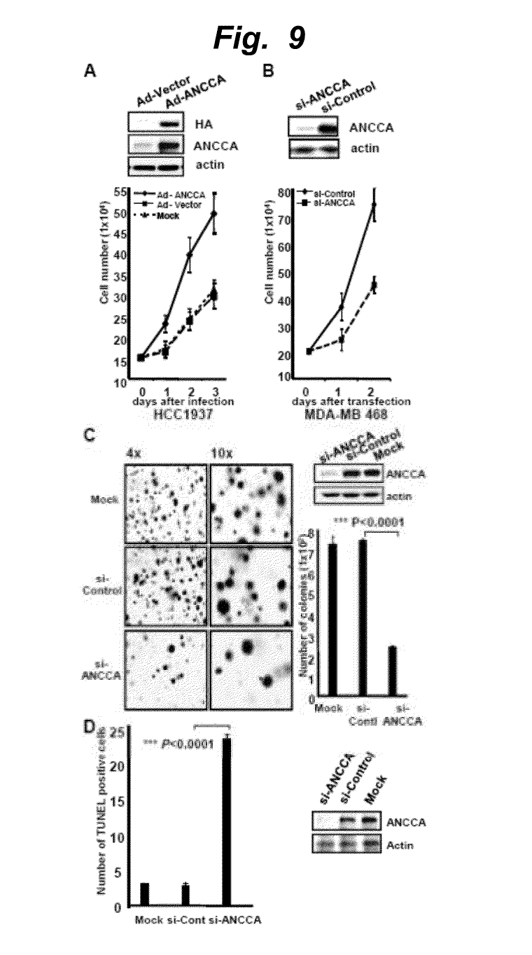

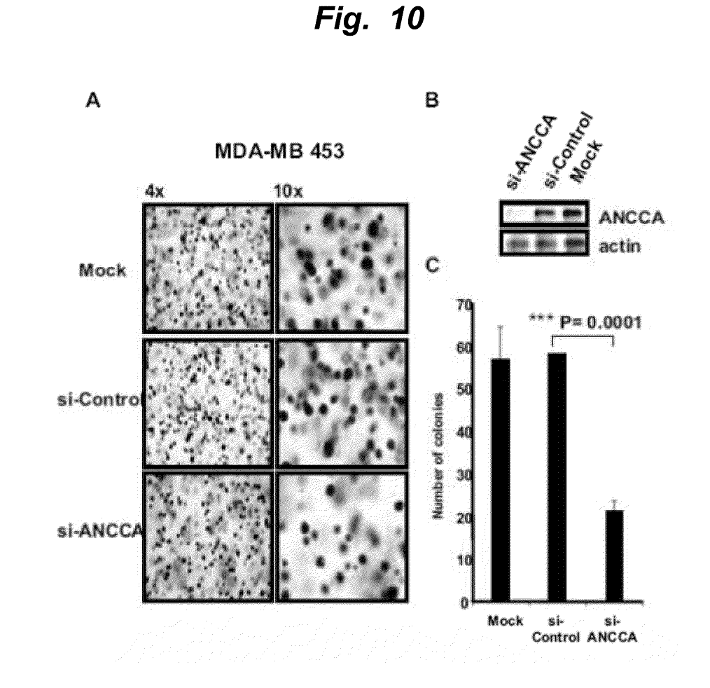

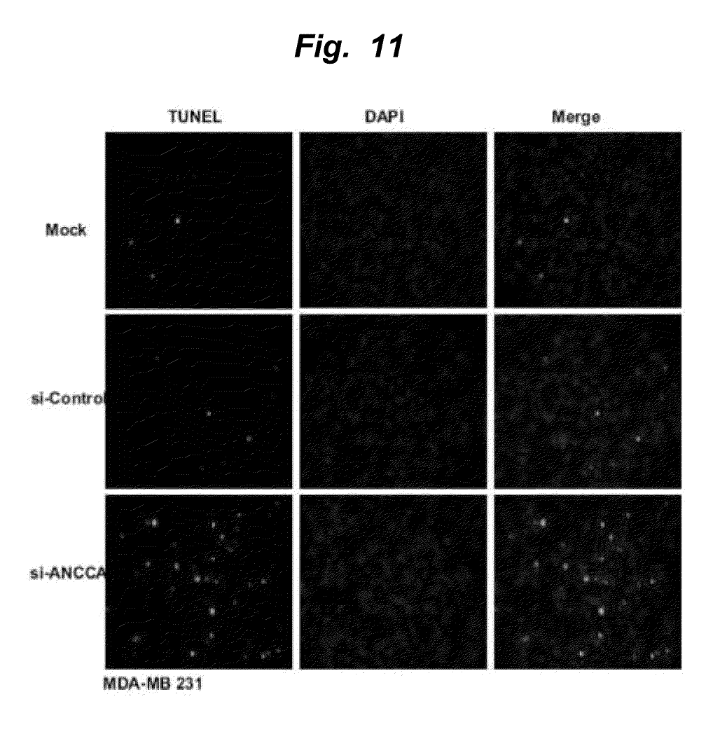

Since expression of ANCCA was particularly high in triple-negative tumors, we examined the role of overexpressed ANCCA in TNBC cells. As shown in FIG. 4A, ANCCA is highly overexpressed in the breast cancer cell lines relative to HMECs. Small interfering RNA (siRNA)-mediated knockdown of ANCCA in MDA-MB-231, -468 and -453 cells showed strong inhibitory effects on their proliferation either in regular 2D culture or in anchorage-independent growth assayed by soft agar colony formation (FIG. 9A, 9C, FIG. 10). In the BRCA1-defective HCC1937 cells that express a moderate level of ANCCA, adenovirus vector-mediated expression of ANCCA led to significantly increased proliferation when compared to cells infected with Adeno-vector control (FIG. 9A). We next examined whether ANCCA was also important for survival of the triple negative cells. MDAMB-231 cells were transfected with siRNA for ANCCA or control sequence and processed for TUNEL assay. As demonstrated in FIG. 9D and FIG. 11, while mock- or control siRNA-transfected cells had very few cells positive for TUNEL staining (<3%), cultures treated with siRNA targeting ANCCA showed a marked increase (approximately 25%) of TUNEL-positive cells. Similar results were obtained with MDA-MB-453 and MDA-MB-468 cells (data not shown). Taken together, these results provide strong evidence that ANCCA overexpression promotes both proliferation and survival of TNBC cells.

ANCCA Controls Important Regulators of Cell Proliferation and Survival Pathways Including EZH2.

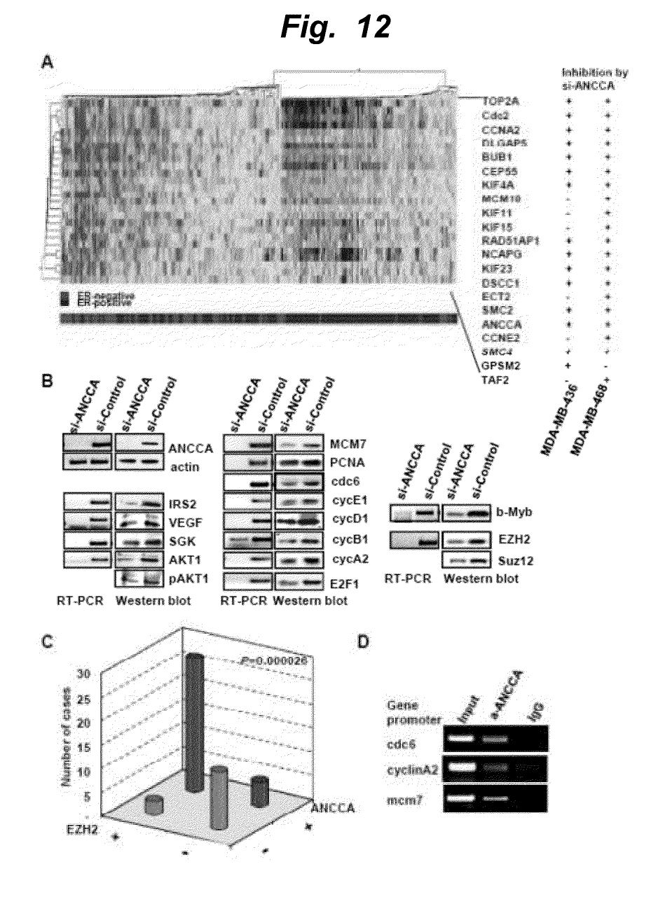

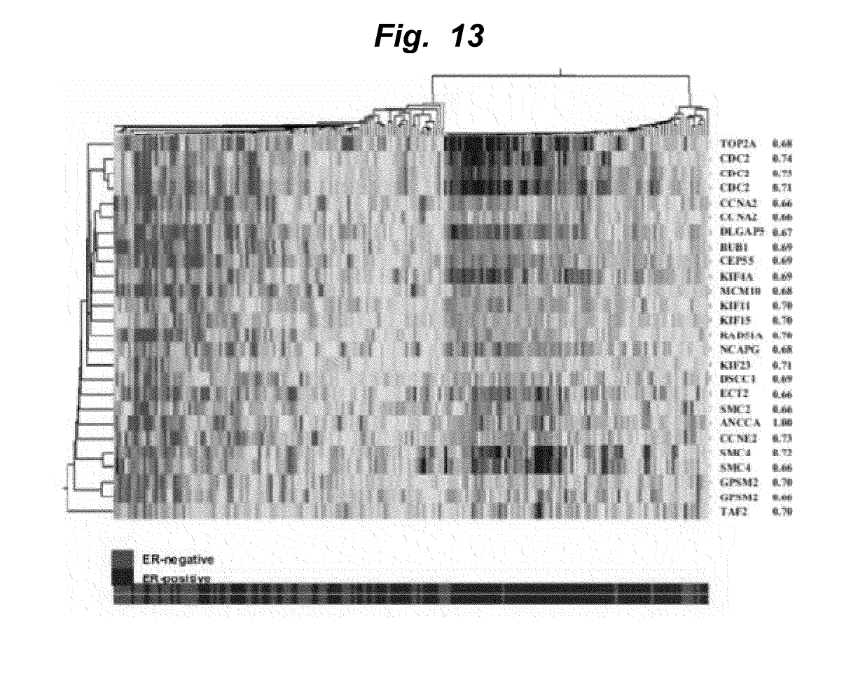

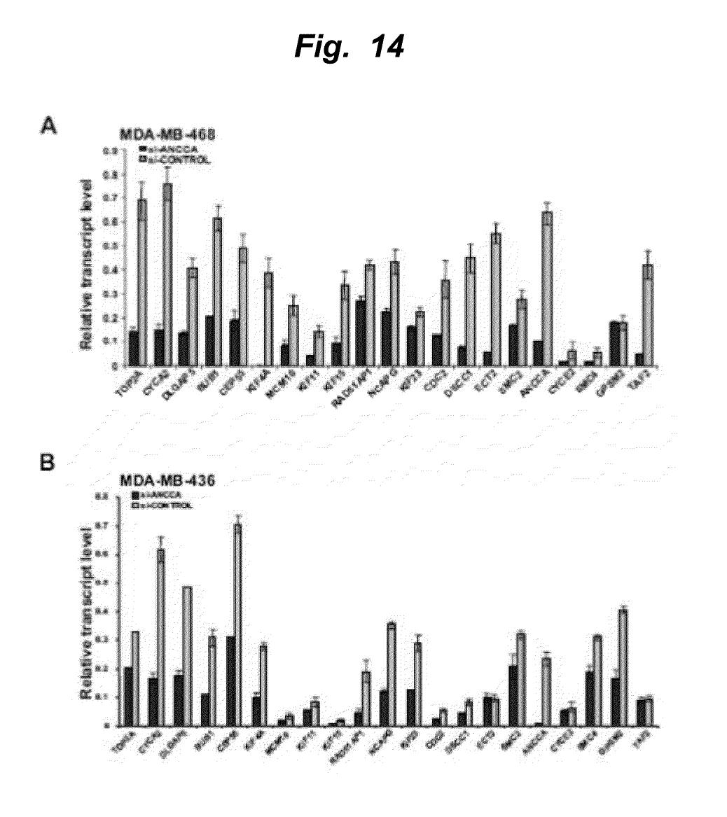

Given the transcriptional coactivator function of ANCCA demonstrated in our previous study (7-9), we analyzed the TRANSBIG Consortium gene expression data set (12), in which ANCCA serves as a prognostic signature gene, for genes having aberrant co-expression associated with overexpressed ANCCA. Strikingly, the analysis identified a group of genes highly enriched in control of cell proliferation and survival with the top 20 genes (25 gene probes) functioning primarily in mitosis (DLGAP5, BUB1, CEP55, KIF4A, KIF11, KIF15, KIF23, DSCC1, ECT2, SMC2, SMC4, GPSM2), DNA replication (MCM10 and Top2A) and cell cycle progression (FIG. 12A, and FIG. 13). Intriguingly, consistent with the results from our IHC study, subsequent hierarchical clustering demonstrated that higher expression of the ANCCA co-expression gene cluster primarily occurred in ERnegative tumors (FIG. 12A, at the left side of the cluster where tumors are primarily ER-negative which is indicated by the red bars at the bottom). ANCCA knockdown in two different TNBC cells demonstrated that they are indeed controlled by ANCCA because their expression is down-regulated upon ANCCA suppression (FIG. 12A, right panel and FIG. 14). Further analysis revealed that expression of proteins with critical roles in survival pathways (e.g., IRS2, SGK1, AKT, VEGFα) as well as phosphorylated AKT was also significantly affected by ANCCA knockdown (FIG. 12B).

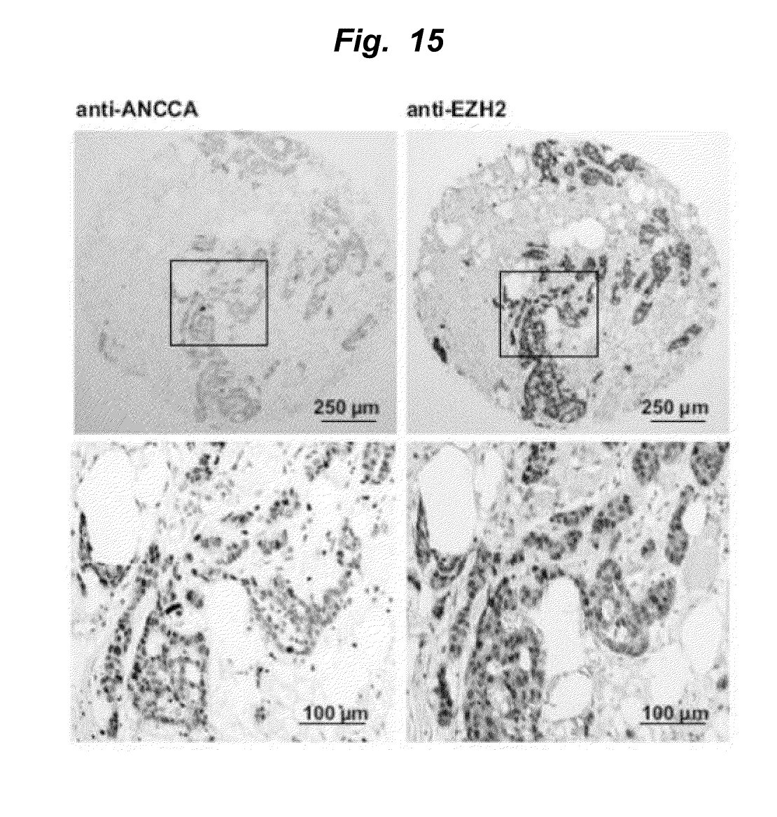

Since overexpression of polycomb group protein (PcG) EZH2 and mitotic regulator B-Myb appear to correlate with high proliferation of basal-like tumors (13, 14), we examined whether ANCCA also controls their expression. Indeed, silencing ANCCA markedly inhibited EZH2 and B-Myb expression at both RNA and protein levels in TNBC cells (FIG. 5B, right panel). The inhibition can also be observed for SUZ12 protein, another component of the Polycomb-repressive complex 2 (PRC2). Therefore, these data demonstrate that EZH2 and B-Myb are both downstream targets of ANCCA. To address the disease relevance of ANCCA control of EZH2, we examined by IHC the expression of ANCCA and EZH2 in a cohort of 48 primary invasive breast carcinoma specimens on a TMA. Consistent with previous findings (15), we found that EZH2 is overexpressed in 62% of the invasive tumors (FIG. 12C and FIG. 15). Importantly, ANCCA overexpression was significantly correlated to the expression of EZH2 in the primary tumors (P<0.000026). These results suggest that de-regulated ANCCA may contribute to EZH2 overexpression in the tumors. Finally, to determine whether ANCCA directly controls the expression of some of the genes identified, we performed ChIP assays. Results in FIG. 12D demonstrate that ANCCA occupies the proximal promoter region of cyclin A2, cdc6 and MCM7. Together, our data indicate that ANCCA is directly involved in control of components of cell proliferation and survival pathways in triple-negative breast cancer cells.

### Discussion

TNBCs, particularly those with basal-like gene expression profiles, tend to show highly elevated mitotic and cell proliferation indices and poor prognosis for long-term survival (1). Aberrations of several key transcriptional regulators (e.g., B-Myb, EZH2 or c-Myc overexpression as well as pRb-E2F deficiency) may underlie the elevated expression of proliferation-associated genes (13, 16). Whether their abnormal functions represent independent events in different tumors or are integrated by other mechanisms is unclear. We initially identified ANCCA as a direct target of the oncogene AIB1/ACTR and as a transcriptional coregulator for ERα and AR to promote the expression of genes driving cancer cell proliferation and survival (8, 9). Recent studies from us and others suggest that ANCCA is overexpressed in many human cancers including breast cancer and may act as a coactivator of c-Myc (8, 9, 17). However, its role in breast cancer progression has been poorly understood. In this study, we examined ANCCA expression in several cohorts of human breast cancer specimens and found that its high levels were significantly associated with poor overall survival and disease recurrence. Interestingly, ANCCA overexpression correlated strongly with the triple-negative subtype of breast cancer. The results of in vitro experiments further demonstrated that high levels of ANCCA were required to maintain proliferation and survival of TNBC cells.

Tumor gene expression signatures have been instrumental in advancing our understanding of the molecular mechanisms underlying the diverse biological phenotypes of breast cancers and now having proven utility for prediction of clinical outcomes, such as tumor responsiveness to chemotherapy (2, 11, 18-23). However, only a few genes seem to be shared across all of the different signatures identified by various studies. Interestingly, ANCCA, which was listed as pro2000, ATAD2 or other undefined names, is one of the few genes that overlap frequently between different signatures. For instance, ANCCA is a component of the 231 genes which gave rise to the 70-gene signature that predicts a clinical outcome of short interval to distant metastasis (11) and is one of the 76 genes identified for prediction of distant metastasis of ER-positive but lymph-node-negative, primary breast cancer, which was later validated in a multi-center study (12, 19, 24, 25). Consistent with our IHC results from an independent cohort of tumors which demonstrate that ANCCA protein levels alone predict tumor grades, high level of ANCCA transcript is a constituent of the genetic grading signature that can reclassify histologic grades of breast cancer (26-28). We also found that ANCCA overexpression strongly correlated with elevated expression of proliferation-associated genes that are often components of the different gene signatures, thereby suggesting that overexpressed ANCCA may either collaborate with them or act upstream as a regulator to stimulate their expression in the tumors. Intriguingly, ANCCA is not a signature gene for the core serum response signature which primarily largely overlaps with cell proliferation genes (29), but instead is part of the PI3K signature (30), suggesting that, in subsets of tumors, aberrant ANCCA may function primarily to promote cancer cell survival. Our data from cell culture studies indicate that both pro-proliferation and pro-survival genes are indeed controlled by ANCCA. Thus, aberrant ANCCA may not only constitute part of the gene signature with prognostic value, but may be a driving force for the altered expression of the signature genes. Moreover, aberrant ANCCA appears to coordinate the tumor signature gene programs.

It is worth noting that in one of the microarray data sets examined (12), the genes most tightly associated with overexpressed ANCCA (i.e., based upon correlation tests) play important roles in mitosis and/or cell proliferation, the majority of which have been shown to be overexpressed in either breast cancer or other types of cancers. When de-regulated, Top2A, cdc2, and cyclin E, as well as the other targets of ANCCA identified in this study (e.g., cdc6, B-Myb and EZH2), can display oncogenic activities. Intriguingly, four members of the human kinesin family (total of 45 genes) were co-expressed with ANCCA overexpression and were validated as ANCCA targets. These kinesins function in different stages of mitosis for spindle assembly and chromosome segregation (31, 32). The aberrant functions of kinesins in cancer render them attractive therapeutic targets (31, 33), or in certain circumstances, may promote cancer cell resistance to taxane-based drugs (34). Together, with the role of ANCCA in mediating expression of pro-survival genes (VEGF, IRS2, SGK and Akt), our data are consistent with the conclusion that overexpressed ANCCA may function as a dominant node for integrating and/or eliciting multiple gene expression programs to promote breast cancer progression. De-regulation of the multiple pathways identified here could constitute part of the molecular basis for the observed association of poor outcome of tumors with highly-elevated ANCCA protein.

Despite our initial identification of ANCCA as a hormone-induced gene (8, 9), we demonstrate here that high levels of ANCCA protein associate most strongly with TNBCs. We also show that high transcript levels of ANCCA tend to associate with ERa-negative or triple-negative status in several data sets from studies of multiple cohorts of tumors (19, 26, 35). Moreover, our unbiased analysis of tumor gene expression datasets also points to the association of high levels of ANCCA with the overexpression of other proliferation genes occurring primarily in ERa-negative tumors. How ANCCA is de-regulated in ERa-negative breast cancers is currently unclear. Results obtained by Ciro and colleagues reported that ANCCA is regulated by the pRb-E2F pathway in fibroblasts and osteosarcoma cells (17). Whether this is the major mechanism for ANCCA overexpression in breast cancer awaits further analysis. Given that one salient feature of triple-negative/basal like tumors is their high proliferation index and functional loss of the pRb-E2F pathway (1, 16, 35, 36), de-regulation of ANCCA through loss of pRb-mediated control is possible. On the other hand, ANCCA may not be merely a downstream target of pRb-E2F, because ANCCA itself mediates expression of multiple Rb-E2F target genes critical for TNBC cell proliferation.

Several transcriptional coregulators including the ones involved in steroid hormone signaling have been strongly implicated in human malignancies (37-42). AIB1 (also known as ACTR/SRC-3) was initially identified as a gene amplified in breast cancer and a coactivator for ERα (43). Later studies revealed that its overexpression does not correlate with ER and PR status and that its aberrant function may include stimulation of tumor growth of both hormone-dependent and -independent breast cancers via ERdependent and -independent pathways (44-48). However, a major distinction between ACTR and ANCCA is that while ACTR overexpression correlates strongly with high HER2 expression, high levels of ANCCA do not, but instead associate with triple-negative status. Although this distinction implies a different mechanism for their aberrant expression in a subset of breast cancers, our recent studies suggest that ANCCA and ACTR may regulate the expression of each other in certain circumstances (7, 8). EZH2, the histone methyltransferase subunit of PRC2 complex is frequently overexpressed in multiple types of cancer (15, 49). Like ANCCA, EZH2 overexpression in breast cancer correlates with high proliferation index and basal-like phenotype and tumor invasiveness (14). ANCCA, however, is unique in that it appears to play a critical role in the control of expression of pro-proliferation genes including EZH2 and B-Myb in triple-negative cancers.

Thus, our data suggest that ANCCA may act as an integrator of several oncogenic transcriptional programs. It will be important to understand whether these transcriptional regulators and coregulators cooperate with each other in the development and progression of breast cancer.

Given our finding that ANCCA is a transcriptional coregulator, it is reasonable to predict that a major functional mode of overexpressed ANCCA in breast cancer is alteration of multiple gene networks including those of pRb-E2F (this study) or c-Myc (17). Our previous study demonstrated that ANCCA is an AAA+ ATPase protein and that its ATPase activity is required for its transcriptional stimulation function (8). ANCCA also possesses a bromodomain that may recognize a distinct histone modification. Given these structure-function features of ANCCA and its aberrant expression in multiple types of human cancers, aberrant ANCCA finds use as a new prognostic marker and a therapeutic target.

### REFERENCES

1. Schneider B P, Winer E P, Foulkes W D, et al. Triple-negative breast cancer: risk factors to potential targets. Clin Cancer Res 2008; 14:8010-8.
2. Perou C M, Sorlie T, Eisen M B, et al. Molecular portraits of human breast tumours. Nature 2000; 406:747-52.
3. Sorlie T, Perou C M, Tibshirani R, et al. Gene expression patterns of breast carcinomas distinguish tumor subclasses with clinical implications. Proc Natl Acad Sci USA 2001; 98:10869-74.
4. Lahusen T, Henke R T, Kagan B L, Wellstein A, Riegel A T. The role and regulation of the nuclear receptor co-activator AIB 1 in breast cancer. Breast Cancer Res Treat 2009; 116:225-37.
5. O'Malley B W, Kumar R. Nuclear receptor coregulators in cancer biology. Cancer Res 2009; 69:8217-22.
6. Xu J, Wu R C, O'Malley B W. Normal and cancer-related functions of the p160 steroid receptor co-activator (SRC) family. Nat Rev Cancer 2009; 9:615-30.
7. Hsia E Y, Kalashnikova E V, Revenko A S, Zou J X, Borowsky A D, Chen H W. Deregulated E2F and the AAA+ Coregulator ANCCA Drive Proto-Oncogene ACTR/AIB1 Overexpression in Breast Cancer. Mol Cancer Res 2010; 8:183-93.
8. Zou J X, Revenko A S, Li L B, Gemo A T, Chen H W. ANCCA, an estrogenregulated AAA+ ATPase coactivator for ERalpha, is required for coregulator occupancy and chromatin modification. Proc Natl Acad Sci USA 2007; 104:18067-72.
9. Zou J X, Guo L, Revenko A S, et al. Androgen-induced coactivator ANCCA mediates specific androgen receptor signaling in prostate cancer. Cancer Res 2009; 69:3339-46.
10. Louie M C, Revenko A S, Zou J X, Yao J, Chen H W. Direct control of cell cycle gene expression by proto-oncogene product ACTR, and its autoregulation underlies its transforming activity. Mol Cell Biol 2006; 26:3810-23.
11. van't Veer L J, Dai H, van de Vijver M J, et al. Gene expression profiling predicts clinical outcome of breast cancer. Nature 2002; 415:530-6.
12. Desmedt C, Piette F, Loi S, et al. Strong time dependence of the 76-gene prognostic signature for node-negative breast cancer patients in the TRANSBIG multicenter independent validation series. Clin Cancer Res 2007; 13:3207-14.
13. Thorner A R, Hoadley K A, Parker J S, Winkel S, Millikan R C, Perou C M. In vitro and in vivo analysis of B-Myb in basal-like breast cancer. Oncogene 2009; 28:742-51.
14. Gonzalez M E, Li X, Toy K, et al. Downregulation of EZH2 decreases growth of estrogen receptor-negative invasive breast carcinoma and requires BRCA1. Oncogene 2009; 28:843-53.
15. Kleer C G, Cao Q, Varambally S, et al. EZH2 is a marker of aggressive breast cancer and promotes neoplastic transformation of breast epithelial cells. Proc Natl Acad Sci USA 2003; 100:11606-11.
16. Herschkowitz J I, He X, Fan C, Perou C M. The functional loss of the retinoblastoma tumour suppressor is a common event in basal-like and luminal B breast carcinomas. Breast Cancer Res 2008; 10:R75.
17. Ciro M, Prosperini E, Quarto M, et al. ATAD2 is a novel cofactor for MYC, overexpressed and amplified in aggressive tumors. Cancer Res 2009; 69:8491-8.
18. van de Vijver M J, He Y D, van't Veer L J, et al. A gene-expression signature as a predictor of survival in breast cancer. N Engl J Med 2002; 347:1999-2009.
19. Wang Y, Klijn J G, Zhang Y, et al. Gene-expression profiles to predict distant metastasis of lymph-node-negative primary breast cancer. Lancet 2005; 365:671-9.
20. Bild A H, Yao G, Chang J T, et al. Oncogenic pathway signatures in human cancers as a guide to targeted therapies. Nature 2006; 439:353-7.
21. Deblois G, Hall J A, Perry M C, et al. Genome-wide identification of direct target genes implicates estrogen-related receptor alpha as a determinant of breast cancer heterogeneity. Cancer Res 2009; 69:6149-57.
22. Bonnefoi H, Potti A, Delorenzi M, et al. Validation of gene signatures that predict the response of breast cancer to neoadjuvant chemotherapy: a substudy of the EORTC 10994/BIG 00-01 clinical trial. Lancet Oncol 2007; 8:1071-8.
23. Li Y, Zou L, Li Q, et al. Amplification of LAPTM4B and YWHAZ contributes to chemotherapy resistance and recurrence of breast cancer. Nat Med 2010; 16:214-8.
24. Foekens J A, Atkins D, Zhang Y, et al. Multicenter validation of a gene expression-based prognostic signature in lymph node-negative primary breast cancer. J Clin Oncol 2006; 24:1665-71.
25. Vale R D. AAA proteins. Lords of the ring. J Cell Biol 2000; 150:F13-9.
26. Ivshina A V, George J, Senko O, et al. Genetic reclassification of histologic grade delineates new clinical subtypes of breast cancer. Cancer Res 2006; 66:10292-301.
27. Ma X J, Salunga R, Tuggle J T, et al. Gene expression profiles of human breast cancer progression. Proc Natl Acad Sci USA 2003; 100:5974-9.
28. Teschendorff A E, Naderi A, Barbosa-Morais N L, et al. A consensus prognostic gene expression classifier for ER positive breast cancer. Genome Biol 2006; 7:R101.
29. Chang H Y, Nuyten D S, Sneddon J B, et al. Robustness, scalability, and integration of a wound-response gene expression signature in predicting breast cancer survival. Proc Natl Acad Sci USA 2005; 102:3738-43.
30. Saal L H, Johansson P, Holm K, et al. Poor prognosis in carcinoma is associated with a gene expression signature of aberrant PTEN tumor suppressor pathway activity. Proc Natl Acad Sci USA 2007; 104:7564-9.
31. Huszar D, Theoclitou M E, Skolnik J, Herbst R. Kinesin motor proteins as targets for cancer therapy. Cancer Metastasis Rev 2009; 28:197-208.
32. Tanenbaum M E, Macurek L, Janssen A, Geers E F, Alvarez-Fernandez M, Medema RH. Kif15 cooperates with eg5 to promote bipolar spindle assembly. Curr Biol 2009; 19:1703-11.
33. Taniwaki M, Takano A, Ishikawa N, et al. Activation of KIF4A as a prognostic biomarker and therapeutic target for lung cancer. Clin Cancer Res 2007; 13:6624-31.
34. De S, Cipriano R, Jackson M W, Stark G R. Overexpression of kinesins mediates docetaxel resistance in breast cancer cells. Cancer Res 2009; 69:8035-42.
35. Gauthier M L, Berman H K, Miller C, et al. Abrogated response to cellular stress identifies DCIS associated with subsequent tumor events and defines basal-like breast tumors. Cancer Cell 2007; 12:479-91.
36. Bosco E E, Wang Y, Xu H, et al. The retinoblastoma tumor suppressor modifies the therapeutic response of breast cancer. J Clin Invest 2007; 117:218-28.
37. Qin L, Liu Z, Chen H, Xu J. The steroid receptor coactivator-1 regulates twist expression and promotes breast cancer metastasis. Cancer Res 2009; 69:3819-27.
38. Ohshiro K, Rayala S K, Kondo S, et al. Identifying the estrogen receptor coactivator PELP1 in autophagosomes. Cancer Res 2007; 67:8164-71.
39. Shen H, Powers N, Saini N, et al. The SWI/SNF ATPase Brm is a gatekeeper of proliferative control in prostate cancer. Cancer Res 2008; 68:10154-62.
40. Torres-Arzayus M I, de Mora J F, Yuan J, et al. High tumor incidence and activation of the PI3K/AKT pathway in transgenic mice define AIB1 as an oncogene. Cancer Cell 2004; 6:263-74.
41. Redmond A M, Bane F T, Stafford A T, et al. Coassociation of estrogen receptor and p160 proteins predicts resistance to endocrine treatment; SRC-1 is an independent predictor of breast cancer recurrence. Clin Cancer Res 2009; 15:2098-106.
42. Hsia E, Zou, J X, and Chen, H. The Roles and Action Mechanisms of p160/SRC Coactivators and the ANCCA Coregulator in Cancer: Burlington: Academic Press; 2009.
43. Anzick S L, Kononen J, Walker R L, et al. AIB1, a steroid receptor coactivator amplified in breast and ovarian cancer. Science 1997; 277:965-8.
44. Yan J, Erdem H, Li R, et al. Steroid receptor coactivator-3/AIB1 promotes cell migration and invasiveness through focal adhesion turnover and matrix metalloproteinase expression. Cancer Res 2008; 68:5460-8.
45. Qin L, Liao L, Redmond A, et al. The AIB1 oncogene promotes breast cancer metastasis by activation of PEA3-mediated matrix metalloproteinase 2 (MMP2) and MMP9 expression. Mol Cell Biol 2008; 28:5937-50.
46. Louie M C, Zou J X, Rabinovich A, Chen H W. ACTR/AIB1 functions as an E2F1 coactivator to promote breast cancer cell proliferation and antiestrogen resistance. Mol Cell Biol 2004; 24:5157-71.
47. Heck S, Rom J, Thewes V, et al. Estrogen-related receptor alpha expression and function is associated with the transcriptional coregulator AIB 1 in breast carcinoma. Cancer Res 2009; 69:5186-93.
48. Fereshteh M P, Tilli M T, Kim S E, et al. The nuclear receptor coactivator amplified in breast cancer-1 is required for Neu (ErbB2/HER2) activation, signaling, and mammary tumorigenesis in mice. Cancer Res 2008; 68:3697-706.
49. Bracken A P, Pasini D, Capra M, Prosperini E, Colli E, Helin K. EZH2 is downstream of the pRB-E2F pathway, essential for proliferation and amplified in cancer. EMBO J. 2003; 22:5323-35.
50. Kalashnikova, et al., Cancer Res. (2010) 70(22):9402-12.
51. Hsia, et al., Adv Drug Deliv Rev. (2010) 62(13):1227-37.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING
SEQ ID NO: 49 - GenBank Ref: NP_054828.2 - amino acid sequence
of ATPase family AAA domain-containing protein 2 [Homo sapiens]

| | |
|---|---|
| 1 | mvvlrsslel hnhsaasatg sldlssdfls lehigrrrlr sagaaqkkpa attakagdgs |
| 61 | svkevetyhr tralrslrkd aqnssdssfe knveiteqla ngrhftrqla rqqadkkkee |
| 121 | hredkvipvt rslrarnivq stehlhedng dvevrrscri rsrysgvnqs mlfdklitnt |
| 181 | aeavlqkmdd mkkmrrqrmr eledlgvfne teesnlnmyt rgkqkdiqrt deettdnqeg |
| 241 | svesseeged qeheddgede ddeddddddd dddddddedd edeedgeeen qkryylrqrk |
| 301 | atvyyqaple kprhqrkpni fysgpaspar pryrlssagp rspyckrmnr rrhaihssds |
| 361 | tsssssedeq hferrrkrsr nrainrclpl nfrkdelkgi ykdrmkigas ladvdpmqld |
| 421 | ssvrfdsvgg lsnhiaalke mvvfpllype vfekfkiqpp rgclfygppg tgktivaral |
| 481 | anecsqgdkr vaffmrkgad clskwvgese rqlrllfdqa yqmrpsiiff deidglapvr |
| 541 | ssrqdqihss ivstllalmd gldsrgeivv igatnrldsi dpalrrpgrf dreflfslpd |
| 601 | kearkeilki htrdwnpkpl dtfleelaen cvgycgadik sicaeaalca lrrrypqiyt |
| 661 | tseklqldls sinisakdfe vamqkmipas qravtspgqa lstvvkpllq ntvdkileal |
| 721 | qrvfphaefr tnktldsdis cpllesdlay sdddvpsvye nglsqksshk akdnfnflhl |
| 781 | nrnacyqpms frprilivge pgfgqgshla pavihalekf tvytldipvl fgvsttspee |
| 841 | tcaqvireak rtapsivyvp hihvwweivg ptlkatfttl lqnipsfapv lllatsdkph |
| 901 | salpeevqel firdygeifn vqlpdkeert kffedlilkq aakppiskkk avlqalevlp |
| 961 | vapppeprsl taeevkrlee qeedtfrelr iflrnvthrl aidkrfrvft kpvdpdevpd |
| 1021 | yvtvikqpmd lssviskidl hkyltvkdyl rdidlicsna leynpdrdpg drlirhraca |
| 1081 | lrdtayaiik eeldedfeql ceeiqesrkk rgcssskyap syyhvmpkqn stlvgdkrsd |
| 1141 | peqneklktp stpvacstpa qlkrkirkks nwylgtikkr rkisqakdds qnaidhkies |
| 1201 | dteetqdtsv dhnetgntge ssveenekqq nasesklelr nnsntcnien eledsrktta |
| 1261 | ctelrdkiac ngdasssqii hisdenegke mcvlrmtrar rsqveqqqli tvekalails |
| 1321 | qptpslvvdh erlknllktv vkksqnynif qlenlyavis qciyrhrkdh dktsliqkme |
| 1381 | qevenfscsr |

SEQ ID NO: 50 - GenBank Ref: NM_014109.3; nucleic acid sequence
of Homo sapiens ATPase family, AAA domain containing 2 (ATAD2), mRNA

| | |
|---|---|
| 1 | cgcggagctt gtggcgccag aattcggagc gcggaagagc cagagctgcg agcgcctgga |
| 61 | gctggatctc tctccggtcg cgcacgccga ggccagtagg gagagaagat ggtggttctc |
| 121 | cgcagcagct tggagctgca caaccactcc gcggcctcgg ccacgggctc cttggacctg |
| 181 | tccagtgact tcctcagtct ggagcacatc ggccggaggc ggctccgctc ggccggcgcg |
| 241 | gcgcagaaga aacccgcggc gaccacagcc aaagcgggcg atgggtcatc agttaaggaa |
| 301 | gttgaaacct accaccggac acgtgcttta agatctttga gaaaagatgc acagaattct |
| 361 | tcagattcta gttttgagaa gaatgtggaa ataacggagc aacttgctaa tggcaggcat |
| 421 | tttacaaggc agttggccag acagcaggct gataaaaaaa aagaagagca cagagaagac |
| 481 | aaagtgattc cagttactcg gtcattgagg gctagaaacatcgttcaaag tacagaacac |
| 541 | ttacatgaag ataatggtga tgttgaagtg cgtcgaagtt gtaggattag aagtcgttat |
| 601 | agtggtgtaa accagtccat gctgtttgac aaacttataa ctaacactgc tgaagctgta |
| 661 | cttcaaaaaa tggatgacat gaagaagatg cgtagacagc gaatgagaga acttgaagac |
| 721 | ttgggagtgt ttaatgaaac agaagaaagc aatcttaata tgtacacaag aggaaaacag |
| 781 | aaagatattc aaagaactga tgaagaaaca actgataatc aagaaggcag tgtggagtca |
| 841 | tctgaagagg gtgaagacca agaacatgaa gatgatggtg aagatgaaga tgatgaagat |
| 901 | gatgatgatg atgacgatga tgatgatgat gatgatgatg aagatgatga agatgaagaa |
| 961 | gatggagaag aagagaatca gaagcgatat tatcttagac agagaaaagc tactgtttac |
| 1021 | tatcaggctc cattggaaaa acctcgtcac cagagaaagc ccaacatatt ttatagtggc |
| 1081 | ccagcttctc ctgcaagacc aagataccga ttatcttccg caggaccaag aagtccttac |
| 1141 | tgtaaacgaa tgaacaggcg aaggcatgca atccacagta gtgactcgac ttcatcttcc |
| 1201 | tcctctgaag atgaacagca ctttgagagg cggaggaaaa ggagtcgtaa tagggctatc |
| 1261 | aataggtgcc tcccactaaa ttttcggaaa gatgaattaa aaggcattta taaagatcga |
| 1321 | atgaaaattg gagcaagcct tgccgatgtt gatccaatgc aactagattc ttcagtacga |
| 1381 | tttgatagtg ttggtggcct gtctaatcat atagcagctc taaaagagat ggtggtgttt |
| 1441 | ccattacttt atccagaagt ctttgaaaaa tttaaaattc aacccccaag aggttgtttg |
| 1501 | ttttatgggc cacctggaac tggaaagact ctggttgcca gagcacttgc caatgagtgc |
| 1561 | agtcaagggg ataaaagagt agcatttttc atgaggaaag gtgctgattg tctaagtaaa |
| 1621 | tgggtaggag aatctgaaag acagctacga ttgctgtttg atcaggccta tcagatgcgc |
| 1681 | ccatcaatta ttttttttga cgaaattgat ggtctggctc cagtacggtc aagcaggcaa |
| 1741 | gatcagattc acagttctat tgtttccacc ctgctagctc ttatggatgg attggacagc |
| 1801 | agaggggaaa ttgtggtcat tggtgctacg aacaggctag attctataga tcctgcttta |
| 1861 | cgaaggcctg gtcgctttga tagagaattc ctctttagcc tgcctgataa agaggctcga |
| 1921 | aaagagattc taaagattca caccagggat tggaatccca aaccactgga cacattttta |
| 1981 | gaagagctag cagaaaactg tgttggatac tgtggagcag atattaaatc aatatgtgct |
| 2041 | gaagctgctt tatgtgcttt acgacgacgctacccacaga tctataccac tagtgagaaa |
| 2101 | ctgcagttgg atctctcttc aattaatatc tcagctaagg atttcgaggt agctatgcaa |
| 2161 | aagatgatac cagcctccca aagagctgtg acatcacctg ggcaggcact gtccaccgtt |
| 2221 | gtgaaaccac tcctgcaaaa cactgttgac aagattttag aagccctgca gagagtattt |
| 2281 | ccacatgcag aattcagaac aaataaaaca ttagactcag atatttcttg tcctctgcta |
| 2341 | gaaagtgact tggcttacag tgatgatgat gttccatcag tttatgaaaa tggactttct |
| 2401 | cagaaatctt ctcataaggc aaaagacaat tttaattttc ttcatttgaa tagaaatgct |
| 2461 | tgttaccaac ctatgtcttt tcgaccaaga atattgatag taggagaacc aggatttggg |
| 2521 | caaggttctc acttggcacc agctgtcatt catgctttgg aaaagtttac tgtatataca |
| 2581 | ttagacattc ctgttctttt tggagttagt actacatccc ctgaagaaac atgtgcccag |
| 2641 | gtgattcgtg aagctaagag aacagcacca agtatagtgt atgttcctca tatccacgtg |
| 2701 | tggtgggaaa tagttggacc gacacttaaa gccacattta ccacattatt acagaatatt |
| 2761 | ccttcatttg ctccagtttt actacttgca acttctgaca aaccccattc cgctttgcca |
| 2821 | gaagaggtgc aagaattgtt tatccgtgat tatggagaga tttttaatgt ccagttaccg |
| 2881 | gataaagaag aacggacaaa attttttgaa gatttaattc taaaacaagc tgctaagcct |
| 2941 | cctatatcaa aaaagaaagc agttttgcag gctttggagg tactcccagt agcaccacca |
| 3001 | cctgagccaa gatcactgac agcagaagaa gtgaaacgac tagaagaaca agaagaagat |
| 3061 | acatttagag aactgaggat tttcttaaga aatgttacac ataggcttgc tattgacaag |
| 3121 | cgattccgag tgtttactaa gcctgttgac cctgatgagg ttcctgatta tgtcactgta |
| 3181 | ataaagcaac caatggacct ttcatctgta atcagtaaaa ttgatctaca caagtatctg |
| 3241 | actgtgaaag actatttgag agatattgat ctaatctgta gtaatgcctt agaatacaat |
| 3301 | ccagatagag atcctggaga tcgtcttatt aggcatagag cctgtgcttt aagagatact |
| 3361 | gcctatgcca taattaaaga agaacttgat gaagactttg agcagctctg tgaagaaatt |
| 3421 | caggaatcta gaaagaaaag aggttgtagc tcctccaaat atgccccgtc ttactaccat |
| 3481 | gtgatgccaa agcaaaattc cactcttgtt ggtgataaaa gatcagaccc agagcagaat |
| 3541 | gaaaagctaa agacaccgag tactcctgtg gcttgcagca ctcctgctca gttgaagagg |
| 3601 | aaaattcgca aaaagtcaaa ctggtactta ggcaccataa aaaagcgaag gaagatttca |
| 3661 | caggcaaagg atgatagcca gaatgccata gatcacaaaa ttgagagtga tacagaggaa |
| 3721 | actcaagaca caagtgtaga tcataatgag accggaaaca caggagagtc ttcggtggaa |
| 3781 | gaaaatgaaa aacagcaaaa tgcctctgaa agcaaactgg aattgagaaa taattcaaat |
| 3841 | acttgtaata tagagaatga gcttgaagac tctaggaaga ctacagcatg tacagaattg |
| 3901 | agagacaaga ttgcttgtaa tggagatgct tctagctctc agataataca tatttctgat |
| 3961 | gaaaatgaag gaaaagaaat gtgtgttctg cgaatgactc gagctagacg ttcccaggta |
| 4021 | gaacagcagc agctcatcac tgttgaaaag gctttggcaa ttctttctca gcctacaccc |
| 4081 | tcacttgttg tggatcatga gcgattaaaa aatcttttga agactgttgt taaaaaaagt |
| 4141 | caaaactaca acatatttca gttggaaaat ttgtatgcag taatcagcca atgtatttat |
| 4201 | cggcatcgca aggaccatga taaaacatca cttattcaga aaatggagca agaggtagaa |
| 4261 | aacttcagtt gttccagatg atgatgtcat ggtatcgagt attctttata ttcagttcct |
| 4321 | atttaagtca tttttgtcat gtccgcctaa ttgatgtagt atgaaaccct gcatctttaa |
| 4381 | ggaaaagatt aaaatagtaa aataaaagta tttaaacttt cctgatattt atgtacatat |
| 4441 | taagataaat gtcatgtgta agataactga taaatattgg aactttgcta gaacaagacc |
| 4501 | ctgtagtaat agtaataata gttgaagttt ggccaactct taataaagtt attttggtaa |
| 4561 | ctaatgtttt atggcactta agaataatta gcagcgttaa attttgtttg tattaagcac |
| 4621 | ttttaatttt atccttccta aaaatagttt attgtatctg acaagaaact tacttaacca |
| 4681 | ttgtgtcctt cccatctttt ttgtcatctt tgttttcttc aaatgccctc ctcccatctg |
| 4741 | ccttgagatt ccctcgtctt cacttaaaag ccagagtgca agtcatgatt tgcgggaggg |
| 4801 | ctcttgaacc acttctggct gcaccacaat tctgtacttg agtatcacag tcattgtttt |
| 4861 | tgagacaaac atttttataa ttctaatttg ggttaataaa gattttaaat atttcttggt |
| 4921 | ttacttttgt aattatatac acaacaaatg tattaataac taccttgtta aacacctttt |
| 4981 | aatagcacaa ggtttttata tttgcaagct gttgatatct ttctaaaact gtttaggtta |
| 5041 | tagtctattg atacttttta tatacaattt tataaatata aatattataa ttttatatta |
| 5101 | atggtaccaa aaatacattt cttaaggtta aaagcatgca cttccatgca tacttgcttt |
| 5161 | tggggagagt ggggagaaga cattctaata atcagtttgt gaaatagctt ctgttggaaa |
| 5221 | ccttttgagg ggaataagga atggtcatct aaaatgagag attctggatt ttaatgcagt |
| 5281 | tcaaagttga gctgtatttt tgttgttgat ttatctggat tttttttaaa gccttctaaa |
| 5341 | acccagtgaa ttcaatacct taattagtac atactatctt atgtaatgca taaagcaatg |
| 5401 | ccagtcactg agaacattta aatatattta tattcctgga gatacacatt ctcatttttg |
| 5461 | ttggtttatt ataaattatt cttctagatg catcttttat aactaggatt tcattttgtg |
| 5521 | tgtatagctt atgtaataaa ttttaaaggt gaaaactctc ttaaaaaaaa aaaaaaaaaa |

What is claimed is:

1. A method of determining the presence of breast cancer in a subject in need thereof comprising
a) measuring the expression level of ATPase family AAA domain-containing protein 2 (ANCCA) in a breast tissue suspected of being cancerous;
b) comparing the expression level of ANCCA in the breast tissue to a threshold level of expression, wherein an expression level of ANCCA that is greater than the threshold level of expression is indicative of the presence of breast cancer in the subject.

2. A method of prognosticating the survival of a subject with breast cancer, comprising
a) measuring the expression level of ANCCA in a breast tissue suspected of being cancerous;
b) comparing the expression level of ANCCA in the breast tissue to a threshold level of expression, wherein an expression level of ANCCA that is greater than the threshold level of expression is indicative of the decreased length of survival of the subject.

3. A method of predicting the presence or occurrence of metastasis in a subject with breast cancer, comprising
a) measuring the expression level of ANCCA in a tissue suspected of being cancerous;
b) comparing the expression level of ANCCA in the tissue to a threshold level of expression, wherein an expression level of ANCCA that is greater than the threshold level of expression is indicative of the presence or occurrence of breast cancer metastasis in the subject.

4. A method of determining the likelihood or risk of recurrence of breast cancer in a subject, comprising:
a) measuring the expression level of ANCCA in a breast tissue suspected of being cancerous or in a breast cancer tumor;
b) comparing the expression level of ANCCA in the breast tissue or breast cancer tumor to a threshold level of expression, wherein an expression level of ANCCA that is greater than the threshold level of expression indicates a relatively higher likelihood or risk of breast cancer recurrence, and wherein an expression level of ANCCA that is less than the threshold level of expression indicates a relatively lower likelihood or risk of breast cancer recurrence.

5. A method of monitoring the progression of a breast cancer, comprising:
a) measuring the expression level of ANCCA in a breast tissue suspected of being cancerous;
b) comparing the expression level of ANCCA in the breast tissue to a threshold level of expression, wherein an expression level of ANCCA that is greater than the threshold level of expression or greater than a previously measured level of expression is indicative of progression of the breast cancer in the subject, and wherein an expression level of ANCCA that is less than the threshold level of expression or less than a previously measured level of expression is indicative of lack of progression, stabilization and/or remission of the breast cancer in the subject.

6. The method of any one of claims 1 to 5, wherein the threshold level of ANCCA expression is equivalent to the level of ANCCA expression in a normal or non-cancerous tissue.

7. The method of any one of claims 1 to 6, wherein the tissue is an epithelial tissue.

8. The method of any one of claims 1 to 7, wherein the tissue is from a biopsy.

9. The method of any one of claims 1 to 8, wherein the tissue suspected of being cancerous is a primary tumor.

10. The method of any one of claims 1 to 9, wherein the subject is exhibiting symptoms of breast cancer.

11. The method of any one of claims 1 to 10, wherein the subject has received a preliminary diagnosis or a diagnosis of breast cancer.

12. The method of any one of claims 1 to 11, wherein the subject has triple-negative breast cancer.

13. The method of any one of claims 1 to 12, wherein the subject is in remission from breast cancer.

14. The method of any one of claims 1 to 13, wherein the subject is human.

15. The method of any one of claims 1 to 14, wherein the protein expression level of ANCCA is measured.

16. The method of claim 15, wherein the ANCCA protein has an amino acid sequence having at least 90% sequence identity to GenBank Accession No. NP_{—}054828.2 (SEQ ID NO:49).

17. The method of claim 15, wherein the protein expression levels of ANCCA protein are measured by immunoblot or immunohistochemistry.

18. The method of any one of claims 1 to 14, wherein the transcription expression level of ANCCA is measured.

19. The method of claim 18, wherein the sequence encoding ANCCA has a nucleic acid sequence having at least 90% sequence identity to GenBank Accession No. NM_{—}014109.3 (SEQ ID NO:50).

20. The method of claim 18, wherein the transcription expression levels of ANCCA are measured by quantitative RT-PCR.

21. The method of any one of claims 1 to 20, further comprising obtaining a biological sample from the subject.

22. The method of any one of claims 1 to 21, further comprising the step of providing a therapeutic or preventative regime to the subject.

23. The method of claim 22, wherein the therapeutic or preventative regime comprises reducing or inhibiting the activity of ANCCA.

24. The method of claim 23, wherein the ATPase activity of ANCCA is reduced or inhibited.

25. The method of claim 24, wherein the expression of ANCCA is reduced or inhibited.

26. The method of claim 25, wherein the expression of ANCCA is inhibited at the transcriptional level.

27. The method of claim 25, wherein the expression of ANCCA is inhibited at the protein level.

28. A method of reducing, inhibiting or preventing the growth or proliferation of a breast cancer cell, comprising contacting the cell with an inhibitory nucleic acid that specifically hybridizes to ANCCA, wherein the expression of ANCCA is reduced by the inhibitory nucleic acid, thereby reducing, inhibiting or preventing the growth or proliferation of the breast cancer cell.

29. The method of claim 28, wherein the breast cancer cell is in vitro.

30. The method of claim 28, wherein the breast cancer cell is in vivo.

31. A method of reducing, inhibiting or preventing the metastasis of a breast in a subject in need thereof, comprising contacting a breast cancer cell or tissue in the subject with an inhibitory nucleic acid that specifically hybridizes to ANCCA, wherein the expression of ANCCA is reduced by the inhibitory nucleic acid, thereby reducing, inhibiting or preventing the metastasis of the breast cancer.

32. The method of any one of claims 28 to 31, wherein the inhibitory nucleic acid specifically binds to a nucleic acid sequence having at least 90% sequence identity to GenBank Accession No. NM_{—}014109.3 (SEQ ID NO:50) or a nucleic acid encoding an amino acid sequence having at least 90% sequence identity to GenBank Accession No. NP_{—}054828.2 (SEQ ID NO:49).

33. The method of any one of claims 28 to 32, wherein the inhibitory nucleic acid is selected from an antisense RNA, a ribozyme, a short inhibitory RNA (siRNA), and a micro RNA (mRNA).

34. The method of any one of claims 28 to 33, wherein the subject has triple-negative breast cancer.

35. The method of any one of claims 28 to 34, wherein the subject is human.


SEQUENCE LISTING (51 sequences)

SEQ ID NO: 1 - DNA, 20 bp, Artificial Sequence
  Description of Artificial Sequence Synthetic primer
tcctatcttttacttgctcc

SEQ ID NO: 2 - DNA, 20 bp, Artificial Sequence
  Description of Artificial Sequence Synthetic primer
caagacttcccaagattatc

SEQ ID NO: 3 - DNA, 20 bp, Artificial Sequence
  Description of Artificial Sequence Synthetic primer
gagtcaaaggttgggttttc

SEQ ID NO: 4 - DNA, 21 bp, Artificial Sequence
  Description of Artificial Sequence Synthetic primer
gtgggaagtgtgtttaactat

SEQ ID NO: 5 - DNA, 20 bp, Artificial Sequence
  Description of Artificial Sequence Synthetic primer
gttggggttatttaaggctg

SEQ ID NO: 6 - DNA, 20 bp, Artificial Sequence
  Description of Artificial Sequence Synthetic primer
tgaataccactggcatgatg

SEQ ID NO: 7 - DNA, 20 bp, Artificial Sequence
  Description of Artificial Sequence Synthetic primer
ttcttccttacctgaatgac

SEQ ID NO: 8 - DNA, 20 bp, Artificial Sequence
  Description of Artificial Sequence Synthetic primer
ttatggtccaattattgtgg

SEQ ID NO: 9 - DNA, 20 bp, Artificial Sequence
  Description of Artificial Sequence Synthetic primer
tttgttgcttgagactcatc

SEQ ID NO: 10 - DNA, 20 bp, Artificial Sequence
  Description of Artificial Sequence Synthetic primer
aaatggaaaacttacctgag

SEQ ID NO: 11 - DNA, 20 bp, Artificial Sequence
  Description of Artificial Sequence Synthetic primer
tttttgcctttcagatcctg

SEQ ID NO: 12 - DNA, 21 bp, Artificial Sequence
  Description of Artificial Sequence Synthetic primer
gaaagagtttcctttgtaagt

SEQ ID NO: 13 - DNA, 20 bp, Artificial Sequence
  Description of Artificial Sequence Synthetic primer
actctgcaccatctggttgg

SEQ ID NO: 14 - DNA, 21 bp, Artificial Sequence
  Description of Artificial Sequence Synthetic primer
catgccatcacagtatctgtt

SEQ ID NO: 15 - DNA, 20 bp, Artificial Sequence
  Description of Artificial Sequence Synthetic primer
gcaaacattactcaaggtat

SEQ ID NO: 16 - DNA, 20 bp, Artificial Sequence
  Description of Artificial Sequence Synthetic primer
ctttctctgaaggaatcatg

SEQ ID NO: 17 - DNA, 21 bp, Artificial Sequence
  Description of Artificial Sequence Synthetic primer
aagggtatttcttgtaaagtc

SEQ ID NO: 18 - DNA, 20 bp, Artificial Sequence
  Description of Artificial Sequence Synthetic primer
aaaagcattggcacttctgg

SEQ ID NO: 19 - DNA, 21 bp, Artificial Sequence
  Description of Artificial Sequence Synthetic primer
aatgagcatgaggatggtgat

SEQ ID NO: 20 - DNA, 20 bp, Artificial Sequence
  Description of Artificial Sequence Synthetic primer
tccgttcaacagtgcccaag

SEQ ID NO: 21 - DNA, 20 bp, Artificial Sequence
  Description of Artificial Sequence Synthetic primer
gcaatagcatctggtggatc

SEQ ID NO: 22 - DNA, 21 bp, Artificial Sequence
  Description of Artificial Sequence Synthetic primer
caagggatggaaaattcagtc

SEQ ID NO: 23 - DNA, 20 bp, Artificial Sequence
  Description of Artificial Sequence Synthetic primer
aaagtgtgaagtacaagtcc

SEQ ID NO: 24 - DNA, 21 bp, Artificial Sequence
  Description of Artificial Sequence Synthetic primer
actaaagtggacagaagttat

SEQ ID NO: 25 - DNA, 20 bp, Artificial Sequence
  Description of Artificial Sequence Synthetic primer
attataccagccaagggatc

SEQ ID NO: 26 - DNA, 21 bp, Artificial Sequence
  Description of Artificial Sequence Synthetic primer
tcctgtacgaaaagaagttag

SEQ ID NO: 27 - DNA, 20 bp, Artificial Sequence
  Description of Artificial Sequence Synthetic primer
gagtcctgaagaaaagaccg

SEQ ID NO: 28 - DNA, 21 bp, Artificial Sequence
  Description of Artificial Sequence Synthetic primer
aagaagtgtggcagcagagtg

SEQ ID NO: 29 - DNA, 20 bp, Artificial Sequence
  Description of Artificial Sequence Synthetic primer
aggacatggtaatggattcc

SEQ ID NO: 30 - DNA, 20 bp, Artificial Sequence
  Description of Artificial Sequence Synthetic primer
tagcagaagtagcagcagcc

SEQ ID NO: 31 - DNA, 20 bp, Artificial Sequence
  Description of Artificial Sequence Synthetic primer
aaggaggtaatgtgtgtttc

SEQ ID NO: 32 - DNA, 21 bp, Artificial Sequence
  Description of Artificial Sequence Synthetic primer
acaatcttgggaatgtgtatc

SEQ ID NO: 33 - DNA, 21 bp, Artificial Sequence
  Description of Artificial Sequence Synthetic primer
agccattagtcactttccaag

SEQ ID NO: 34 - DNA, 20 bp, Artificial Sequence
  Description of Artificial Sequence Synthetic primer
cacagcagcctaaaagatac

SEQ ID NO: 35 - DNA, 20 bp, Artificial Sequence
  Description of Artificial Sequence Synthetic primer
ctttacaaagcccactttaa

SEQ ID NO: 36 - DNA, 20 bp, Artificial Sequence
  Description of Artificial Sequence Synthetic primer
agagtgctgatttagaagaa

SEQ ID NO: 37 - DNA, 24 bp, Artificial Sequence
  Description of Artificial Sequence Synthetic primer
cccccagaagtagcagagtttgtg

SEQ ID NO: 38 - DNA, 24 bp, Artificial Sequence
  Description of Artificial Sequence Synthetic primer
gctttgtcccgtgactgtgtagag

SEQ ID NO: 39 - DNA, 21 bp, Artificial Sequence
  Description of Artificial Sequence Synthetic primer
gagaaaatctggcaccacacc

SEQ ID NO: 40 - DNA, 21 bp, Artificial Sequence
  Description of Artificial Sequence Synthetic primer
atacccctcgtagatgggcac

SEQ ID NO: 41 - DNA, 23 bp, Artificial Sequence
  Description of Artificial Sequence Synthetic primer
gaaatcccatcaccatcttccag

SEQ ID NO: 42 - DNA, 24 bp, Artificial Sequence
  Description of Artificial Sequence Synthetic primer
atgagtccttccacgataccaaag

SEQ ID NO: 43 - DNA, 22 bp, Artificial Sequence
  Description of Artificial Sequence Synthetic primer
actgactgctgctgccttgtgc

SEQ ID NO: 44 - DNA, 22 bp, Artificial Sequence
  Description of Artificial Sequence Synthetic primer
tcggtggtgtcataatgcctcc

SEQ ID NO: 45 - DNA, 22 bp, Artificial Sequence
  Description of Artificial Sequence Synthetic primer
caccgagtactcctgtggcttg

SEQ ID NO: 46 - DNA, 26 bp, Artificial Sequence
  Description of Artificial Sequence Synthetic primer
tctagctcgagtcattcgcagaacac

SEQ ID NO: 47 - DNA, 19 bp, Artificial Sequence
  Description of Artificial Sequence Synthetic primer
gcacttggcttcaaagctg

SEQ ID NO: 48 - DNA, 24 bp, Artificial Sequence
  Description of Artificial Sequence Synthetic primer
ccaagtatttcttcagatccatgg

SEQ ID NO: 49 - protein, 1390 aa, Homo sapiens
MVVLRSSLELHNHSAASATGSLDLSSDFLSLEHIGRRRLRSAGAAQKKPAATTAKAGDGSSVKEVETYHRTRALRSLRKDAQNSSDSSFEKNVEITEQLANGRHFTRQLARQQADKKKEEHREDKVIPVTRSLRARNIVQSTEHLHEDNGDVEVRRSCRIRSRYSGVNQSMLFDKLITNTAEAVLQKMDDMKKMRRQRMRELEDLGVFNETEESNLNMYTRGKQKDIQRTDEETTDNQEGSVESSEEGEDQEHEDDGEDEDDEDDDDDDDDDDDDDDEDDEDEEDGEEENQKRYYLRQRKATVYYQAPLEKPRHQRKPNIFYSGPASPARPRYRLSSAGPRSPYCKRMNRRRHAIHSSDSTSSSSSEDEQHFERRRKRSRNRAINRCLPLNFRKDELKGIYKDRMKIGASLADVDPMQLDSSVRFDSVGGLSNHIAALKEMVVFPLLYPEVFEKFKIQPPRGCLFYGPPGTGKTLVARALANECSQGDKRVAFFMRKGADCLSKWVGESERQLRLLFDQAYQMRPSIIFFDEIDGLAPVRSSRQDQIHSSIVSTLLALMDGLDSRGEIVVIGATNRLDSIDPALRRPGRFDREFLFSLPDKEARKEILKIHTRDWNPKPLDTFLEELAENCVGYCGADIKSICAEAALCALRRRYPQIYTTSEKLQLDLSSINISAKDFEVAMQKMIPASQRAVTSPGQALSTVVKPLLQNTVDKILEALQRVFPHAEFRTNKTLDSDISCPLLESDLAYSDDDVPSVYENGLSQKSSHKAKDNFNFLHLNRNACYQPMSFRPRILIVGEPGFGQGSHLAPAVIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

SEQ ID NO: 50 - DNA, 5580 bp, Homo sapiens
cgcggagcttgtggcgccagaattcggagcgcggaagagccagagctgcgagcgcctggagctggatctctctccggtcgcgcacgccgaggccagtagggagagaagatggtggttctccgcagcagcttggagctgcacaaccactccgcggcctcggccacgggctccttggacctgtccagtgacttcctcagtctggagcacatcggccggaggcggctccgctcggccggcgcggcgcagaagaaacccgcggcgaccacagccaaagcgggcgatgggtcatcagttaaggaagttgaaacctaccaccggacacgtgctttaagatctttgagaaaagatgcacagaattcttcagattctagttttgagaagaatgtggaaataacggagcaacttgctaatggcaggcattttacaaggcagttggccagacagcaggctgataaaaaaaaagaagagcacagagaagacaaagtgattccagttactcggtcattgagggctagaaacatcgttcaaagtacagaacacttacatgaagataatggtgatgttgaagtgcgtcgaagttgtaggattagaagtcgttatagtggtgtaaaccagtccatgctgtttgacaaacttataactaacactgctgaagctgtacttcaaaaaatggatgacatgaagaagatgcgtagacagcgaatgagagaacttgaagacttgggagtgtttaatgaaacagaagaaagcaatcttaatatgtacacaagaggaaaacagaaagatattcaaagaactgatgaagaaacaactgataatcaagaaggcagtgtggagtcatctgaagagggtgaagaccaagaacatgaagatgatggtgaagatgaagatgatgaagatgatgatgatgatgacgatgatgatgatgatgatgatgatgaagatgatgaagatgaagaagatggagaagaagagaatcagaagcgatattatcttagacagagaaaagctactgtttactatcaggctccattggaaaaacctcgtcaccagagaaagcccaacatattttatagtggcccagcttctcctgcaagaccaagataccgattatcttccgcaggaccaagaagtccttactgtaaacgaatgaacaggcgaaggcatgcaatccacagtagtgactcgacttcatcttcctcctctgaagatgaacagcactttgagaggcggaggaaaaggagtcgtaatagggctatcaataggtgcctcccactaaattttcggaaagatgaattaaaaggcatttataaagatcgaatgaaaattggagcaagccttgccgatgttgatccaatgcaactagattcttcagtacgatttgatagtgttggtggcctgtctaatcatatagcagctctaaaagagatggtggtgtttccattactttatccagaagtctttgaaaaatttaaaattcaacccccaagaggttgtttgttttatgggccacctggaactggaaagactctggttgccagagcacttgccaatgagtgcagtcaaggggataaaagagtagcatttttcatgaggaaaggtgctgattgtctaagtaaatgggtaggagaatctgaaagacagctacgattgctgtttgatcaggcctatcagatgcgcccatcaattattttttttgacgaaattgatggtctggctccagtacggtcaagcaggcaagatcagattcacagttctattgtttccaccctgctagctcttatggatggattggacagcagaggggaaattgtggtcattggtgctacgaacaggctagattctatagatcctgctttacgaaggcctggtcgctttgatagagaattcctctttagcctgcctgataaagaggctcgaaaagagattctaaagattcacaccagggattggaatcccaaaccactggacacatttttagaagagctagcagaaaactgtgttggatactgtggagcagatattaaatcaatatgtgctgaagctgctttatgtgctttacgacgacgctacccacagatctataccactagtgagaaactgcagttggatctctcttcaattaatatctcagctaaggatttcgaggtagctatgcaaaagatgataccagcctcccaaagagctgtgacatcacctgggcaggcactgtccaccgttgtgaaaccactcctgcaaaacactgttgacaagattttagaagccctgcagagagtatttccacatgcagaattcagaacaaataaaacattagactcagatatttcttgtcctctgctagaaagtgacttggcttacagtgatgatgatgttccatcagtttatgaaaatggactttctcagaaatcttctcataaggcaaaagacaattttaattttcttcatttgaatagaaatgcttgttaccaacctatgtcttttcgaccaagaatattgatagtaggagaaccaggatttgggcaaggttctcacttggcaccagctgtcattcatgctttggaaaagtttactgtatatacattagacattcctgttctttttggagttagtactacatcccctgaagaaacatgtgcccaggtgattcgtgaagctaagagaacagcaccaagtatagtgtatgttcctcatatccacgtgtggtgggaaatagttggaccgacacttaaagccacatttaccacattattacagaatattccttcatttgctccagttttactacttgcaacttctgacaaaccccattccgctttgccagaagaggtgcaagaattgtttatccgtgattatggagagatttttaatgtccagttaccggataaagaagaacggacaaaattttttgaagatttaattctaaaacaagctgctaagcctcctatatcaaaaaagaaagcagttttgcaggctttggaggtactcccagtagcaccaccacctgagccaagatcactgacagcagaagaagtgaaacgactagaagaacaagaagaagatacatttagagaactgaggattttcttaagaaatgttacacataggcttgctattgacaagcgattccgagtgtttactaagcctgttgaccctgatgaggttcctgattatgtcactgtaataaagcaaccaatggacctttcatctgtaatcagtaaaattgatctacacaagtatctgactgtgaaagactatttgagagatattgatctaatctgtagtaatgccttagaatacaatccagatagagatcctggagatcgtcttattaggcatagagcctgtgctttaagagatactgcctatgccataattaaagaagaacttgatgaagactttgagcagctctgtgaagaaattcaggaatctagaaagaaaagaggttgtagctcctccaaatatgccccgtcttactaccatgtgatgccaaagcaaaattccactcttgttggtgataaaagatcagacccagagcagaatgaaaagctaaagacaccgagtactcctgtggcttgcagcactcctgctcagttgaagaggaaaattcgcaaaaagtcaaactggtacttaggcaccataaaaaagcgaaggaagatttcacaggcaaaggatgatagccagaatgccatagatcacaaaattgagagtgatacagaggaaactcaagacacaagtgtagatcataatgagaccggaaacacaggagagtcttcggtggaagaaaatgaaaaacagcaaaatgcctctgaaagcaaactggaattgagaaataattcaaatacttgtaatatagagaatgagcttgaagactctaggaagactacagcatgtacagaattgagagacaagattgcttgtaatggagatgcttctagctctcagataatacatatttctgatgaaaatgaaggaaaagaaatgtgtgttctgcgaatgactcgagctagacgttcccaggtagaacagcagcagctcatcactgttgaaaaggctttggcaattctttctcagcctacaccctcacttgttgtggatcatgagcgattaaaaaatcttttgaagactgttgttaaaaaaagtcaaaactacaacatatttcagttggaaaatttgtatgcagtaatcagccaatgtatttatcggcatcgcaaggaccatgataaaacatcacttattcagaaaatggagcaagaggtagaaaacttcagttgttccagatgatgatgtcatggtatcgagtattctttatattcagttcctatttaagtcatttttgtcatgtccgcctaattgatgtagtatgaaaccctgcatctttaaggaaaagattaaaatagtaaaataaaagtatttaaactttcctgatatttatgtacatattaagataaatgtcatgtgtaagataactgataaatattggaactttgctagaacaagaccctgtagtaatagtaataatagttgaagtttggccaactcttaataaagttattttggtaactaatgttttatggcacttaagaataattagcagcgttaaattttgtttgtattaagcacttttaattttatccttcctaaaaatagtttattgtatctgacaagaaacttacttaaccattgtgtccttcccatcttttttgtcatctttgttttcttcaaatgccctcctcccatctgccttgagattccctcgtcttcacttaaaagccagagtgcaagtcatgatttgcgggagggctcttgaaccacttctggctgcaccacaattctgtacttgagtatcacagtcattgtttttgagacaaacatttttataattctaatttgggttaataaagattttaaatatttcttggtttacttttgtaattatatacacaacaaatgtattaataactaccttgttaaacaccttttaatagcacaaggtttttatatttgcaagctgttgatatctttctaaaactgtttaggttatagtctattgatactttttatatacaattttataaatataaatattataattttatattaatggtaccaaaaatacatttcttaaggttaaaagcatgcacttccatgcatacttgcttttggggagagtggggagaagacattctaataatcagtttgtgaaatagcttctgttggaaaccttttgaggggaataaggaatggtcatctaaaatgagagattctggattttaatgcagttcaaagttgagctgtatttttgttgttgatttatctggattttttttaaagccttctaaaacccagtgaattcaataccttaattagtacatactatcttatgtaatgcataaagcaatgccagtcactgagaacatttaaatatatttatattcctggagatacacattctcatttttgttggtttattataaattattcttctagatgcatcttttataactaggatttcattttgtgtgtatagcttatgtaataaattttaaaggtgaaaactctcttaaaaaaaaaaaaaaaaaa

SEQ ID NO: 51 - DNA, 9 bp, Artificial Sequence
  Description of Artificial Sequence Synthetic oligonucleotide
ttcaagaga